(19) United States
(12) Patent Application Publication
(10) Pub. No.: US 2024/0002430 A1
(43) Pub. Date: Jan. 4, 2024

(54) METHOD FOR PRODUCING PROTEIN COMPOSITIONS

(71) Applicant: HOFFMAN-LA ROCHE INC., Little Falls, NJ (US)

(72) Inventors: Roberto FALKENSTEIN, Penzberg (DE); Frank WEDEKIND, Penzberg (DE); Martin REMPT, Penzberg (DE); Michael LEISS, Penzberg (DE); Franziska EDELMANN, Penzberg (DE); Tobias GRAF, Penzberg (DE)

(73) Assignee: HOFFMAN-LA ROCHE INC., Little Falls, NJ (US)

(21) Appl. No.: 18/020,048

(22) PCT Filed: Aug. 6, 2021
(86) PCT No.: PCT/EP2021/072039

§ 371 Date: Feb. 6, 2023

(30) Foreign Application Priority Data
Aug. 7, 2020 (EP) 20190136.0

(51) Int. Cl. C07K 1/04; C07K 1/22

(52) U.S. Cl. CPC C07K 1/04; C07K 1/22

(57) ABSTRACT

The present invention generally relates to the provision of means and methods for preparing protein compositions. The invention provides a method comprising the steps of (i) contacting a starting composition comprising a protein with a hydrolase inhibitor, wherein the hydrolase inhibitor is immobilized on a solid carrier; (ii) recovering a composition comprising the protein. Furthermore, the invention relates to a method for preparing a protein formulation comprising the steps of (i) contacting a starting composition comprising a protein with a hydrolase inhibitor, wherein the hydrolase inhibitor is immobilized on a solid carrier; (ii) recovering a composition comprising the protein, wherein the method for preparing the protein formulation further comprises adding a surfactant, preferably a fatty acid ester, to the composition comprising the protein. Moreover, the invention relates to a protein composition obtained or obtainable by the methods described herein and/or to a protein formulation obtained or obtainable by the methods described herein.

The present invention generally relates to the provision of means and methods for preparing protein compositions. The invention provides a method comprising the steps of
(i) contacting a starting composition comprising a protein with a hydrolase inhibitor, wherein the hydrolase inhibitor is immobilized on a solid carrier;
(ii) recovering a composition comprising the protein.

Furthermore, the invention relates to a method for preparing a protein formulation comprising the steps of
(i) contacting a starting composition comprising a protein with a hydrolase inhibitor, wherein the hydrolase inhibitor is immobilized on a solid carrier;
(ii) recovering a composition comprising the protein,

wherein the method for preparing the protein formulation further comprises adding a surfactant, preferably a fatty acid ester, to the composition comprising the protein.

Moreover, the invention relates to a protein composition obtained or obtainable by the methods described herein and/or to a protein formulation obtained or obtainable by the methods described herein.

Recombinant biopharmaceutical proteins (e.g. monoclonal antibodies (mAbs), antibody fragments (Fab), complex, antibody derived proteins and fusion proteins) are usually expressed in Chinese hamster ovary (CHO) host cell lines with high expression rates (Durocher (2009) Curr. Opin. Biotech. 20:700-707). During purification of these proteins, the amount of host cell proteins (HCP) needs to be eliminated or at least reduced adequately to warrant patient safety for clinical or marketed application of the drug (Vanderlaan (2018) Biotechnol Prog. 34:828-837). HCPs with hydrolytic activity can lead to surfactant degradation (Labrenz (2014) J. Pharm. Sci. 103:2268-2277). Surfactant degradation of enzymatic origin has been observed in multiple biopharmaceutical formulations within the biopharmaceutical industry and may result in visible particle formation due to reduced surfactant mediated prevention of protein aggregation and/or due to accumulating surfactant break down products (Labrenz (2014) J. Pharm. Sci. 103:2268-2277; Cao (2015) J. Pharm. Sci. 104:433-446; Larson (2020) J. Pharm. Sci. 109:633-639; Graf (2020) Eur. J. Pharm. Biopharm. 152:318-326). Particle and/or aggregate formation in turn leads to shelf-life restriction.

Recent studies have unveiled several hydrolytic enzymes, which are attributed to increased surfactant degradation in biopharmaceutical formulations, including lipoprotein lipase (LPL) (Chiu (2017) Biotechnol. Bioeng. 114:1006-1015), lysosomal phospholipase A2 (LPLA2) (Hall (2016) J. Pharm. Sci. 105:1633-1642) and putative phospholipase B-like 2 (PLBL2) (Dixit (2016) J. Pharm. Sci. 105:1657-1666). Most notably, the persistence of these enzymes along downstream processing has been shown repeatedly underlining their hard-to-remove and/or mAb associating properties (Valente (2014) Biotechnol. Bioeng. 112:1232-1242; Levy (2014) Biotechnol. Bioeng. 111:904-912; Tran (2016) J. Chromatogr. A 1438:31-38.)

The selective removal of individual HCPs, including hydrolytic enzymes, such as lipases, is poorly understood. It was observed that the majority of aggregates/visible particles containing fatty acids, derived from the degradation of surfactants like Polysorbate 20 or 80, is increasing during (long-term) storage (Labrenz (2014) J. Pharm. Sci. 103: 2268-2277; Tomlinson (2015) Mol. Pharmaceutics 12: 3805-3815). This triggered the development of strategies to eliminate the contamination that initiates Polysorbate degradation. Based on stability studies as well as lipase activity assays the performance of wash and/or incubation steps during purification was assessed for HCP removal and it was found that a residual risk remains that particles are generated during the (long-term) storage studies of protein formulations containing a polysorbate. WO2016/057739 for example used a hydrophobic interaction medium to reduce lipases. However, said method comes with the disadvantage that hydrophobic interaction chromatography relies on differences in the molecular characteristics (hydrophobicity). The suitability of this approach may vary depending on the properties of the protein. Moreover, depending on the hydrophobicity of the protein and the applied buffer conditions, the protein could bind to the HIC resin or lead to a potential yield loss of the product.

Accordingly, there is a need for means and methods to reduce the hydrolytic activity in protein compositions.

The technical problem underlying the present invention is the provision of means and methods for preparing protein compositions with increased/higher stability and/or for reducing the hydrolytic activity in protein compositions. The technical problem is solved and the above mentioned needs are addressed by the provision of the embodiments characterized in the claims and as provided herein below.

The invention relates to a method comprising the steps of
(i) contacting a starting composition comprising a protein with a hydrolase inhibitor, wherein the hydrolase inhibitor is immobilized on a solid carrier;
(ii) recovering a composition comprising the protein.

As illustrated in the appended examples it has been surprisingly and unexpectedly found that a hydrolase inhibitor can be immobilized on a solid carrier without losing functionality (i.e. the immobilized inhibitor retains binding to the hydrolase). Thereby hydrolases can be reliably removed from protein compositions.

Example 1 demonstrates that immobilized hydrolase inhibitor Orlistat B retains the capability to interact with Lysosomal Lipase A2 (LPLA2). An antibody solution was spiked with recombinant LPLA2 and subsequently incubated with Orlistat B immobilized on magnetic beads. After incubation, the beads were subjected to LC-MS/MS analysis showing a high affinity of LPLA2 towards the beads (FIG. 2B). Furthermore, it was shown that the antibody solution incubated with Orlistat B immobilized on beads shows a pronounced reduction in hydrolytic activity compared to an antibody solution incubated with control beads without Orlistat B (FIG. 2A). This result further confirms that LPLA2 is removed from the antibody solution upon incubation with immobilized Orlistat B. Accordingly, it is envisaged without necessarily being bound by scientific theory that Orlistat B although covalently bound to beads is still able to form a covalent bond with LPLA2.

Example 2 confirms this result for an antibody solution of higher concentration (FIG. 3). This shows that removal of hydrolases and a reduction of hydrolytic activity can be achieved for a wide range of protein concentrations in compositions.

Example 3 shows for a mixed mode anion exchange (MMAEX) load solution of an antibody composition (an anti-CD20/anti-CD3 bispecific antibody (glofitamab, also known as RG6026)) that there is indeed residual hydrolase activity. It is demonstrated that Orlistat B immobilized on a solid carrier is capable of reducing the hydrolase activity in the recovered protein composition compared to the starting protein composition (FIG. 4). There is also a reduction of the hydrolase activity compared to the respective controls (beads with linker and without immobilized Orlistat B/beads without linker) demonstrating a specific effect of hydrolase inhibitor Orlistat B on the reduction of hydrolase activity. Moreover, Example 3 shows that Orlistat B retains its capacity to specifically bind to a hydrolase when immobilized via two different linking methods, namely either the method using the reaction between an amine and an N-Hydroxysuccinimide (NHS)-activated ester or the method using the affinity of Streptavidin and biotin. Thus, the reduction in hydrolytic activity can be achieved independent of a specific immobilization method and/or for various proteins of interest.

Example 4 further confirms the removal/reduction of residual hydrolase activity by immobilized Orlistat in a trastuzumab conditioned protein A elution pool (FIG. 5). Moreover, Example 4 also shows that the hydrolase inhibitor Orlistat A can be used to remove hydrolases from an antibody solution, i.e. that hydrolase inhibitors as such can be immobilized on various solid carriers (NHS-activated sepharose beads and Streptavidin Sepharose beads, respectively) and retain their binding activity, which can, in turn, be used advantageously to reduce the hydrolytic activity.

Example 5 confirms with a yet further hydrolase inhibitor (bis-enol-ester) that immobilization to a solid carrier can be achieved while retaining specific binding activity to the hydrolase (FIG. 6). Example 5 further confirms that the hydrolase indeed specifically binds to the inhibitor and not unspecifically to the bead material.

In summary, it is illustrated by the present invention that hydrolases can be removed from several different starting antibody compositions with different inhibitors immobilized on different solid carriers. Said compositions also have various antibody concentrations. All in all, this demonstrates that the present invention can be advantageously practiced under a wide variety of conditions.

Hydrolases are known to degrade their respective substrates (e.g. fatty acid esters/lipids, such as surfactants as used routinely in protein formulations, like pharmaceutical compositions) and thereby lead to (visible) particle formation due to reduced surfactant mediated prevention of protein aggregation and/or due to accumulating surfactant break down products. Hence, it is anticipated herein that a reduction of the hydrolytic activity by the present invention leads to less particle(s) (formation) and/or improved stability/solubility of the protein formulation.

One already known molecule, the so-called Orlistat, is able to inhibit most of the enzymes related to hydrolysis of lipids, and in consequence surfactants that have similar chemical properties (Jahn (2020), Pharm. Res. 37:118). The molecule is capable of mimicking the natural substrate of lipases and therefore is capable of catching lipases via the formation of a covalent ester bond at the active site (Fako (2014) ACS Catal. 4: 3444-3453). However, it is not possible to simply supplement the protein composition with Orlistat or other inhibitors because most lipase inhibitors are non-polar compounds and must be added in an organic solvent. Especially Orlistat is highly insoluble in water. Additionally, these chemicals can induce adverse events in patients during application of the drug and can impact drug quality. Especially Orlistat can be toxic when applied intravenously e.g. together with the protein composition. Furthermore, the ester bond between Orlistat and the enzyme may be hydrolyzed (acidic or base catalyzed) during storage of the protein composition re-liberating the active enzyme.

During storage of a protein composition to which a hydrolase inhibitor (e.g. Orlistat) has been added (i.e. wherein the inhibitor is not immobilized on a solid carrier), the hydrolase inhibitor (e.g. Orlistat) may unspecifically and covalently react with e.g. serine residues of the protein (e.g. antibodies) due to the high concentration of the protein in the protein composition. Said unspecific reaction may decrease the binding affinity of the protein (e.g. antibodies) to the target (i.e. decreased product efficacy) and/or may lead to (increased) aggregation of the protein (i.e. decreased product quality).

By contrast, when the protein composition is only contacted with the immobilized hydrolase inhibitor in accordance with the invention for a short time said unspecific reactions are less likely. In the rare case that the immobilized hydrolase inhibitor reacts with the protein of interest in the protein composition during step (i) (“contacting a starting composition comprising a protein with a hydrolase inhibitor, wherein the hydrolase inhibitor is immobilized on a solid carrier”) the protein of interest will also be immobilized to the solid carrier and will not be present in the recovered protein composition. Thus, by employing the herein described method of the invention, the mentioned disadvantages occurring when a hydrolase inhibitor (e.g. Orlistat) is added to a protein composition can be avoided or minimized (i.e. the presence of (a) protein(s) (e.g. an antibody/antibodies) with decreased binding affinity to the target (i.e. decreased product efficacy) and/or (increased) aggregation of (a) protein(s) (e.g. an antibody/antibodies) protein (i.e. decreased product quality).

As a further advantage of the present invention, the reduction in hydrolytic activity can be achieved without the presence of the hydrolase inhibitor as such in the protein formulation and/or without the need to remove such a hydrolase inhibitor (i.e. in case the inhibitor was added into the composition without immobilization to a solid carrier).

The hydrolase inhibitor may covalently bind the hydrolase. The solid carrier on which the hydrolase inhibitor is immobilized may be used several times/repeatedly for carrying out the herein described method, particularly step (i) thereof. This may be useful for example if step (i) is carried out repeatedly, as described further below. Thus, a starting composition can be subjected to the same solid carrier repeatedly/subsequently, for example until the maximum binding capacity for the hydrolase is reached. However, in a preferred aspect the material, i.e. the solid carrier on which the hydrolase inhibitor is immobilized is for single use/utilization. Single use/utilization of said material may be preferred in purification processes/platforms that are e.g. GMP (Good Manufacturing Practice) certified and/or commercial purification processes/platforms. However, for basic research and/or academic research it is envisaged that said material can be utilized two times or multiple times. Utilization of said material two times or multiple times as used herein means that the step (i) contacting a starting composition comprising a protein with a hydrolase inhibitor, wherein the hydrolase inhibitor is immobilized on a solid carrier is performed two times or multiple times with the identical hydrolase inhibitor immobilized on a solid carrier. The skilled person is well aware under which circumstances the solid carrier with the immobilized hydrolase inhibitor can be utilized two times or multiple times, e.g. when after the first utilization (i.e. contacting a starting composition comprising a protein with a hydrolase inhibitor, wherein the hydrolase inhibitor is immobilized on a solid carrier) the hydrolase inhibitor molecules have not quantitatively reacted with hydrolases, i.e. when there are still hydrolase inhibitor molecules on the solid carrier that are able to react with hydrolases in a second or subsequent utilization. In other words, the material, i.e. the solid carrier with the immobilized hydrolase inhibitor may be used/utilized two times or multiple times when not all hydrolase inhibitor molecules have adsorbed (bound) a hydrolase after the first utilization (i.e. contacting a starting composition comprising a protein with a hydrolase inhibitor, wherein the hydrolase inhibitor is immobilized on a solid carrier). That means that also in a second or subsequent utilization hydrolases can adsorb to the hydrolase inhibitor. In other words, there are still “free” (unbound) hydrolase inhibitor molecules that can adsorb hydrolase molecules in a second or subsequent utilization.

As mentioned above the removal of HCPs by hydrophobic interaction chromatography may depend on the properties of the protein in the protein formulation. On the contrary, hydrolase inhibitors are directly targeted against present hydrolases and thus generally remain unaffected by the protein in the protein formulation. Accordingly, the present invention uses a specific ligand to remove specific impurities from the protein formulation/composition.

In the following the invention is described in more detail.

In particular the invention relates to the following items:
1. A method comprising the steps of
   (i) contacting a starting composition comprising a protein with a hydrolase inhibitor, wherein the hydrolase inhibitor is immobilized on a solid carrier;
   (ii) recovering a composition comprising the protein.
2. The method of item 1, wherein the method is for preparing a composition comprising a protein.
3. The method of item 1 or 2, wherein the composition comprising the protein has reduced hydrolytic activity compared to the starting composition and/or has a reduced content of a hydrolase compared to the starting composition, for example, as determined by a lipase activity assay (lipase enzymatic assay for polysorbates (LEAP) assay) and/or a fatty acid by mass-spectrometry (FAMS) assay.
4. The method of any one of items 1 to 3, wherein the method is for reducing the hydrolytic activity in the starting composition and/or for reducing the content of a hydrolase in the starting composition.
5. The method of any one of items 1 to 4, wherein the starting composition further comprises (a) hydrolase(s) and/or has hydrolytic activity.
6. The method of any one of items 1 to 5, wherein the protein composition is prepared by removing a hydrolase from the starting composition and/or by reducing the content of a hydrolase in the starting composition.
7. The method of any one of items 1 to 6, wherein said step (i) further comprises a step (a) adsorbing the hydrolase to the hydrolase inhibitor.
8. The method of any one of items 1 to 7, wherein the composition comprising a protein is a solution comprising a protein.
9. The method of item 8, wherein the solution is an aqueous solution.
10. The method of item 8 or 9, wherein the solution is a buffered solution.
11. The method of any one of items 1 to 10, wherein the hydrolase(s) is/are (an) esterase(s) and/or (a) amidase(s).
12. The method of item 11, wherein the esterase(s) is/are (a) carboxylic ester hydrolase(s) and/or (a) thioesterase(s).
13. The method of item 12, wherein the carboxylic ester hydrolase(s) is/are (a) lipase(s).
14. The method of any one of items 1 to 12, wherein the hydrolase(s) is/are selected from the group consisting of Lipoprotein Lipase, Palmitoyl Proteinthioesterase, Acid Ceramidase, the C-terminal domain of Fatty Acid synthase, Putative Phospholipase b-like 2, Lysosomal Acid Lipase and Lysosomal Phospholipase.
15. The method of any one of items 1 to 14, wherein the immobilized inhibitor is selected from the group consisting of orlistat or bis-enol-ester.
16. The method of any one of items 1 to 15, wherein the solid carrier is selected from the group consisting of sepharose, polystyrene and smart polymers.
17. The method of any one of items 1 to 16, wherein the inhibitor is immobilized on a solid carrier via the reaction of an azido group and an alkyne group.
18. The method of any one of items 1 to 16, wherein the inhibitor is immobilized on a solid carrier via the binding of a streptavidin group to a biotin group.
19. The method of any one of items 1 to 16, wherein the inhibitor is immobilized on a solid carrier via the reaction of an amino group and an N-Hydroxysuccinimid group.
20. The method of any one of items 1 to 19, wherein the inhibitor is a group obtainable by reacting a compound comprising or consisting of formula (1), (2), (3) or (4), preferably formula (1), (2) or (4) with an azide

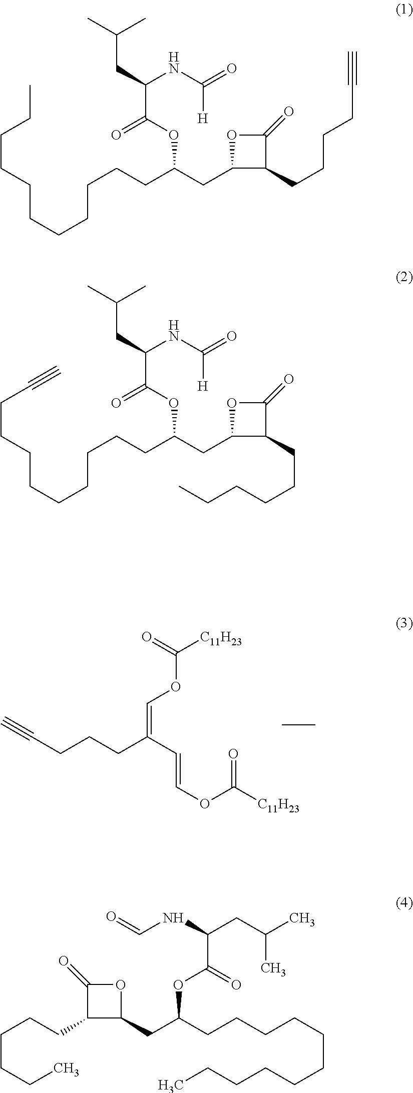

21. The method of any one of items 1 to 20, wherein the steps are carried out in the following order: step (i) followed by step (ii).
22. The method of any one of items 1 to 21, further comprising, prior to and/or after step (i) and/or prior to and/or after step (ii), the steps of protein preparation and/or purification.
23. The method of any one of items 1 to 22, wherein step (i) is carried out after affinity chromatography, preferably after a Protein A affinity chromatography.
24. The method of any one of items 1 to 23, wherein the protein is an antibody.
25. The method of item 24, wherein the antibody is a monoclonal antibody.
26. The method of item 24 or 25, wherein the antibody is a human or a humanized antibody.
27. The method of any one of items 1 to 26, wherein the protein is an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A antibody, an antiVEGF-A/ANG2 antibody, an anti-CD79b antibody, an anti-ST2 antibody, an anti-factor D antibody, an anti-factor IX antibody, an anti-factor X antibody, an anti-abeta antibody, an antitau antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, a FAP-IL2v fusion protein, ocrelizumab, pertuzumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, lebrikizumab, omalizumab ranibizumab, emicizumab, selicrelumab, prasinezumab, glofitamab, simlukafusp alfa, and RG7827.
28. A method for preparing a protein formulation, the method comprising the steps of the method of any one of items 1 to 27, wherein the method for preparing the protein formulation further comprises adding a surfactant, preferably a fatty acid ester, to the composition.
29. A method for preparing a protein formulation, wherein the method for preparing the protein formulation further comprises adding a surfactant, preferably a fatty acid ester, to the composition obtained by or obtainable by the method of any one of items 1 to 27.
30. The method of item 28 or 29, wherein the protein formulation is (essentially) free of visible and/or subvisible particles.
31. The method of any one of items 28 to 30, wherein the protein formulation is stable for at least 6 months, at least 12 months, at least 18 months, at least 24 months, at least 36 months or at least 60 months under the recommended storage conditions.
32. The method of any one of items 28 to 30, wherein the fatty acid ester is polyoxyethylene sorbitan or iso-sorbide fatty acid mono-, di- or tri-ester.
33. The method of any one of items 28 to 32, wherein the fatty acid ester is Polyoxyethylene (20) sorbitan laurate.
34. The method of any one of items 28 to 32, wherein the fatty acid ester is selected from the group group consisting of polysorbate 20, polysorbate 40, polysorbate 60, polysorbate 61, polysorbate 65, polysorbate 80, polysorbate 81, polysorbate 85 or polysorbate 120, or a combination thereof.
35. The method of any one of items 28 to 32 wherein the fatty acid ester is polysorbate 20 or polysorbate 80.
36. The method of any one of items 28 to 31 wherein the surfactant is Mono- or Di-acylglycerol, saccharide-fatty acid ester or α-tocopheryl Polyethylene glycol (PEG) succinate.
37. The method of any one of items 28 to 36, wherein the degradation of the fatty acid ester is less than 20%, preferably less than 10% and more preferably less than 5% within 24 months.
38. The method of any one of items 26 to 37, wherein the antibody concentration is at least 1 mg/ml and up to 250 mg/ml.
39. The method of any one of items 28 to 38, further comprising adding a buffer, excipient, diluent, stabilizer and/or carrier to the composition.
40. The method of any one of items 28 to 39, wherein the protein formulation is a pharmaceutical composition.
41. The method of any one of items 1 to 40, wherein the composition comprising the protein and/or the protein formulation is essentially free of hydrolase inhibitors.
42. A protein composition obtained by or obtainable by the method of any one of items 1 to 27.
43. A protein formulation obtained by or obtainable by the method of any one of items 28 to 41.
44. The protein formulation of item 43 for use as a medicament or for use in medicine.
45. Use of a hydrolase inhibitor immobilized on a solid carrier for preparing a protein composition and/or a protein formulation.
46. Use of a hydrolase inhibitor immobilized on a solid carrier for removing or reducing hydrolytic activity in a protein composition and/or in a protein formulation.
47. Use of a hydrolase inhibitor immobilized on a solid carrier for removing or reducing (content of) impurities, particularly of host cell proteins, and preferably of (a) hydrolase(s) in a protein composition and/or in a protein formulation.
48. Use of a hydrolase inhibitor immobilized on a solid carrier for inhibiting or reducing (the formation of) visible and/or sub-visible particles and/or the occurrence/presence of surfactant degradants in a protein composition and/or in a protein formulation.

Further, the invention relates to the following aspects:
1. A method comprising the steps of
   (i) contacting a starting composition comprising a protein with a hydrolase inhibitor, wherein the hydrolase inhibitor is immobilized on a solid carrier;
   (ii) recovering a composition comprising the protein.
2. The method of item 1, wherein the composition comprising the protein has reduced hydrolytic activity compared to the starting composition.
3. The method of item 1 or 2, wherein the protein composition is prepared by removing (a) hydrolase(s) from the starting composition.
4. The method of item 3, wherein the hydrolase(s) is/are (an) esterase(s) and/or (a) amidase(s), preferably wherein the esterase(s) is/are (a) carboxylic ester hydrolase(s) and/or (a) thioesterase(s).
5. The method of item 4, wherein the esterase(s) is/are (a) lipase(s).
6. The method of item 3, wherein the hydrolase(s) is/are selected from the group consisting of Lipoprotein Lipase, Palmitoyl Proteinthioesterase, Acid Ceramidase, Fatty Acid synthase, Putative Phospholipase b-like 2, Lysosomal Acid Lipase and Lysosomal Phospholipase.
7. The method of any one of items 1 to 6, wherein the immobilized inhibitor is selected from the group consisting of orlistat or bis-enol-ester.
8. The method of any one of items 1 to 7, wherein the inhibitor is obtainable by reacting a compound comprising or consisting of formula (1), (2), (3) or (4), preferably formula (1), (2) or (4) with an azide:

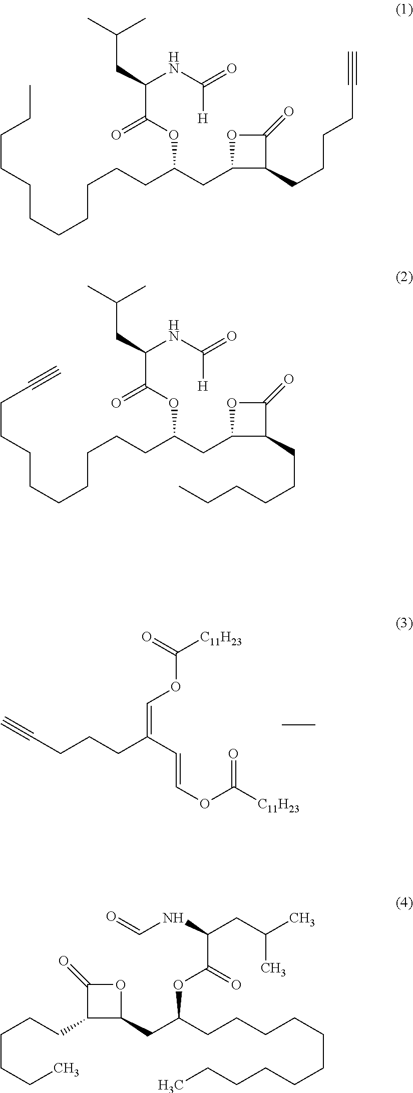

9. The method of any one of items 1 to 8, further comprising, prior to and/or after step (i), the steps of protein preparation and/or purification.
10. The method of any one of items 1 to 9, wherein step (i) is carried out after an affinity chromatography, preferably after a Protein A affinity chromatography.
11. The method of any one of items 1 to 10, wherein the protein is an antibody.
12. A method for preparing a protein formulation, the method comprising the steps of the method of any one of items 1 to 11, wherein the method for preparing the protein formulation further comprises adding a surfactant, preferably a fatty acid ester, to the composition.
13. The method of item 12, wherein the protein formulation is (essentially) free of visible and/or subvisible particles.
14. The method of item 12 or 13, wherein the fatty acid ester is polyoxyethylene sorbitan or iso-sorbide fatty acid mono-, di- or tri-ester.
15. A protein composition obtained by or obtainable by the method of any one of items 1 to 11.

As mentioned above, the invention relates to a method comprising the steps of
(i) contacting a starting composition comprising a protein with a hydrolase inhibitor, wherein the hydrolase inhibitor is immobilized on a solid carrier;
(ii) recovering a composition comprising the protein.

The term “starting composition comprising a protein” as used herein generally refers to a composition comprising a “protein of interest”. In this context, the term “protein” refers to a “protein of interest”. The “protein of interest” typically is a protein that is to be obtained from a cell culture/culture cells and that is to be isolated and/or to be purified from the cell culture/culture cells, for example, for commercial, pharmaceutical, diagnostic and/or therapeutic purposes and/or for scientific research. The term “protein of interest” typically refers to one specific protein of interest, e.g. a specific antibody binding to antigen A. However, the term “protein of interest” can also refer to one or more different “proteins of interest, for example to two or more different proteins of interest (e.g. one antibody binding to antigen A and a second antibody binding to antigen B). Moreover, as explained further below, the “starting composition” may, in addition to the “protein of interest”, comprise one or more host cell protein(s) (HCP(s).

The meaning of the term “protein” is well known in the art and is used accordingly in context of the present invention. In particular, the term “protein” as used herein means, in accordance with the present invention, a protein or a polypeptide, which encompasses amino acid chains of a given length, wherein the amino acid residues are linked by covalent peptide bonds. Typically, a “protein” has a length of at least 20 contiguous amino acids and up to 3500 contiguous amino acids. The protein can be a monomer. Included within the term “protein” is also a protein forming multimers, such as dimers, trimers, tetramers etc. wherein such multimers can be homo- or hetero-multimers. Thus, the term “protein” as used herein, can be such a multimer (multimeric protein) composed of monomeric proteins. Exemplary proteins/proteins of interest are described further herein below.

The term “starting composition comprising a protein” (or likewise “initial composition comprising a protein”) as used herein is specifically meant to refer to an extract from a cell culture or culture cells, said cell culture or culture cells producing the protein (protein of interest) or said cell culture or culture cells used to produce the protein (protein of interest). The extract may be a crude extract or may be a purified extract (a purified extract has been subject to one or more purification steps). The “starting composition comprising a protein” may accordingly already have been subject to one or more conventional purification steps, such as ion exchange chromatography (e.g. anion exchange chromatography or cation exchange chromatography), ultrafiltration/diafiltration, viral filtration, size exclusion chromatography, affinity chromatography and hydrophobic interaction chromatography, mixed mode chromatography/multimodal chromatography (e.g. mixed mode ion exchange chromatography) and any combination thereof.

While the herein provided method can be used for practically any “composition comprising a protein” (even highly purified compositions with no or only trace amounts of any impurities), it is understood that the “starting composition comprising a protein” as used herein normally comprises at least one impurity from the cell culture/culture cells used to produce said protein/protein of interest, i.e. at least one impurity from the cell culture/culture cells from which the protein/protein of interest is obtained/prepared. The impurity may be one or more host cell protein(s), specifically (a) protein(s) with hydrolytic activity (hydrolase).

For example, a starting composition comprising a protein may be prepared by homogenizing cells, which grow in a cell culture to produce said protein of interest, in a homogenizing solution. The starting composition comprising a protein may also be the cell culture supernatant when the protein is directly secreted during cell culture. Accordingly, the starting composition comprising a protein may be Harvested Cell Culture Fluid (HCCF). HCCF is a composition (as) separated (by filtration) from cells, cell debris and/or aggregates, e.g. HCCF is obtained after conditioning and/or filtration (e.g. by filtration of the composition prepared from the cell culture or of the cell culture supernatant). The starting composition herein preferably is Harvested Cell Culture Fluid (HCCF). The composition that becomes HCCF after filtration may be referred to as pre-harvest cell culture fluid (PHCCF). In other words, the composition (e.g. prepared from the cell culture or of the cell culture supernatant) prior to conditioning and/or filtration is referred to as pre-harvest cell culture fluid (PHCCF).

The starting composition comprising the protein may be contacted with the hydrolase inhibitor directly after homogenizing said cells, which grow in a cell culture to produce the protein of interest. However, it is also envisaged herein that the starting composition comprising the protein is subjected to one or more purification steps before said composition comprising the protein is contacted with the hydrolase inhibitor. Non-limiting examples of such purification steps are described herein below.

In accordance with the invention, the “starting composition comprising a protein” is subject to a (further) purification comprising contacting the “starting composition comprising a protein” with a hydrolase inhibitor, wherein the hydrolase inhibitor is immobilized on a solid carrier (step (i) of the method).

After that step, a further step is performed (step (ii) of the method), namely “recovering a composition comprising the protein”, i.e. a “composition comprising the protein” is recovered. More simply phrased, the further step is “recovering the protein”. In this context, the term “protein” refers to a “protein of interest” as defined above. In other words, the protein of interest comprised in the starting composition also is the protein of interest in the recovered composition or is the recovered protein of interest. We also refer herein to that composition as the “recovered composition” and to that protein as the “recovered protein”.

It is understood that the “starting composition comprising a protein” is contacted with a hydrolase inhibitor immobilized on a solid carrier. In other words, the term “contacting a starting composition comprising a protein with a hydrolase inhibitor, wherein said hydrolase inhibitor is immobilized on a solid carrier” means “contacting a starting composition, said composition comprising a protein, with a hydrolase inhibitor, wherein said hydrolase inhibitor is immobilized on a solid carrier”.

It is envisaged that the hydrolase inhibitor may be immobilized on the solid carrier prior to the contacting step, i.e. prior to step (i) of the method. In other word the hydrolase inhibitor may be immobilized on the solid carrier (attached to the solid carrier) before it is brought into contact with the starting composition comprising a protein. It is understood that herein the hydrolase inhibitor is immobilized on the solid carrier prior to the contacting step. In other words the hydrolase inhibitor is immobilized on the solid carrier (attached to the solid carrier) before it is brought into contact with the starting composition comprising a protein.

Accordingly, the invention relates in a preferred aspect to a method comprising the steps of
(i) contacting a starting composition comprising a protein with a hydrolase inhibitor immobilized on a solid carrier;
(ii) recovering a composition comprising the protein.

In other words, the invention relates in a preferred aspect to a method comprising the steps of
(i) contacting a starting composition comprising a protein with a hydrolase inhibitor, wherein the hydrolase inhibitor is a hydrolase inhibitor immobilized on a solid carrier;
(ii) recovering a composition comprising the protein.

It is understood that by “contacting a starting composition comprising a protein with a hydrolase inhibitor” the above mentioned impurity, such as one or more host cell protein(s), specifically (a) protein(s) with hydrolytic activity (hydrolase), if present in the starting composition, is removed or at least its content reduced in the “recovered composition” compared to the “starting composition comprising a protein”. It is preferred herein that a host cell protein(s), specifically (a) protein(s) with hydrolytic activity (hydrolase) is present in the starting composition.

As mentioned, the term “recovering a composition comprising the protein” may be used synonymously with “recovering the protein”. The method thus aims at preparing (or likewise obtaining) a composition comprising the protein. In this context, the “composition comprising a protein” is normally the recovered composition according to step (ii) of the method.

Accordingly, the invention provides a method for preparing or for obtaining a composition comprising a protein comprising the steps of
(i) contacting a starting composition comprising a protein with a hydrolase inhibitor, wherein the hydrolase inhibitor is immobilized on a solid carrier;
(ii) recovering a composition comprising the protein.

Thus, a “composition comprising a protein” is normally prepared or obtained if steps (i) and (ii) are carried out (and, optionally, repetitions of steps (i) and (ii), as explained above). However, the method may also comprise additional steps (such as further purification steps (e.g. conventional purification) prior to step (i) and/or after step (ii) and/or between steps (i) and (ii) in order to prepare or obtain the “composition comprising a protein”. The same explanations and definitions apply when steps (i) and (ii) are repeated, as explained above.

As also discussed herein above, the aim of the method of the present invention is to remove or reduce the hydrolytic activity (if present) in the starting composition and/or to remove or reduce the content of impurities (if present), particularly of host cell proteins, and preferably of (a) hydrolase(s) in the starting composition. Thus, in one aspect, the “composition comprising the protein” has reduced hydrolytic activity compared to the starting composition and/or has a reduced content of impurities (if present), particularly of host cell proteins, and preferably of (a) hydrolase(s) compared to the starting composition. The same explanations and definitions apply when steps (i) and (ii) are repeated, as explained herein. It is preferred herein that hydrolytic activity is present in the starting composition and/or that impurities are present in the starting composition, particularly that host cell proteins, and preferably (a) hydrolase(s) are present in the starting composition.

Accordingly, in one aspect, the invention provides a method comprising the steps of
(i) contacting a starting composition comprising a protein with a hydrolase inhibitor, wherein the hydrolase inhibitor is immobilized on a solid carrier;
(ii) recovering a composition comprising the protein,

wherein the composition comprising the protein (the recovered composition) has reduced hydrolytic activity compared to the starting composition

and/or wherein the composition comprising the protein (the recovered composition) has a reduced content of impurities, particularly of host cell proteins, and preferably of (a) hydrolase(s) compared to the starting composition.

In this context it is understood that the starting composition has hydrolytic activity and/or that the starting composition has/comprises impurities, particularly host cell proteins, and preferably (a) hydrolase(s). In other words, hydrolytic activity and/or impurities as defined above are preferably present in the starting composition. The presence of hydrolytic activity and/or of impurities as defined above in the starting composition can be determined by assays known in the art and/or disclosed and provided herein. Non-limiting examples for measuring, determining or quantifying hydrolytic activity are an assay to detect lipolytic activity or, in other words, a lipase activity assay (e.g. as described by Jahn et al.), herein termed LEAP (lipase enzymatic assay for polysorbases) assay (Jahn (2020) Pharm. Res. 37: 118), and/or a fatty acid by mass spectrometry (FAMS) assay (Honemann (2019) J. Chromatogr. B 1116: 1-8; Cheng (2019) J. Pharm. Sci. 108: 2880-2886). Such assays can also be used to determine a reduction of hydrolytic activity.

For determination the presence or amount of a protein, routine methods can be employed herein, such as immunoagglutination, immunoprecipitation (e.g. immunodiffusion, immunelectrophoresis, immune fixation), western blotting techniques (e.g. (in situ) immuno histochemistry, (in situ) immuno cytochemistry, affinity chromatography, enzyme immunoassays), and the like. These and other suitable methods of contacting proteins are well known in the art and are, for example, also described in Sambrook and Russell (2001.) Molecular cloning: a laboratory manual. Vol. 2, 3rd edn. Cold Spring Harbor Laboratory Press, New York. Quantification can be performed by taking advantage of the techniques referred to above, in particular Western blotting techniques. Generally, the skilled person is aware of methods for the quantitation of proteins. Amounts of purified protein in solution can be determined by physical methods, e.g. photometry. Methods of quantifying particular proteins in a mixture rely on specific binding, e.g of antibodies. Specific detection and quantitation methods exploiting the specificity of antibodies comprise for example immunohistochemistry (in situ). Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction.

As explained further below, an indirect parameter for determining that a given composition, e.g. the starting composition, has hydrolytic activity and/or has/comprises impurities as defined above, is the occurrence/presence of visible and/or sub-visible particles (e.g. due to insoluble matter of surfactant degradants) and/or the occurrence/presence of surfactant degradants as such. Likewise, a reduction in visible and/or sub-visible particles (e.g. due to insoluble matter of surfactant degradants) and/or the occurrence/presence of surfactant degradants as such is an indirect parameter for determining that a given composition (preferably the composition comprising the protein (the recovered composition)) has reduced hydrolytic activity and/or has a reduced content of impurities as defined herein compared to the starting composition.

That determination (e.g. of the occurrence/presence of visible and/or sub-visible particles (e.g. due to insoluble matter of surfactant degradants) and/or the occurrence/presence of surfactant degradants as such) can involve storage of the given composition (e.g. the starting composition and/or the composition comprising the protein (the recovered composition)), for example storage under long term conditions as explained further below. For example, if a given composition does not show the occurrence/presence of visible and/or sub-visible particles (e.g. due to insoluble matter of surfactant degradants) and/or the occurrence/presence of surfactant degradants as such at the start, but does show same after storage, it is indicated that the given composition at the start has hydrolytic activity and/or has/comprises impurities as defined above.

This can be used to determine the presence of hydrolytic activity and/or impurities in a “starting composition” as used herein as a reference point, for example, if that composition is not contacted with an inhibitor. By comparing the same with a “starting composition” that is contacted with an inhibitor (i.e. the composition comprising the protein (the recovered composition)) it can be assessed whether the contacted composition (i.e. the composition comprising the protein (the recovered composition)) has reduced hydrolytic activity and/or has a reduced content of impurities as defined herein compared to the reference starting composition (and hence to the “starting composition”).

The meaning of the term “hydrolytic activity” is well known in the art, is used accordingly in context of the present invention and described in detail further below.

“Hydrolase inhibitor” is used herein in the broadest sense and may refer to a molecule that inhibits another molecule with hydrolytic activity. The definitions of hydrolytic activity and hydrolases are provided herein below. Non-limiting examples of hydrolases are also provided herein below. The hydrolase inhibitor may inhibit the enzyme by binding to the enzyme. The hydrolase inhibitor may bind to the active site of the enzyme. The hydrolase inhibitor may form a covalent bond with the enzyme. Non-limiting examples of hydrolase inhibitors, which form a covalent bond with the corresponding enzyme and which are used in context of the present invention are Orlistat and bis-enol-ester.

The following relates to a “hydrolase inhibitor” provided and to be used in accordance with the present invention. The terms “hydrolase inhibitor” and “inhibitor of hydrolase” are used interchangeably herein.

The terms “hydrolase inhibitor” and “inhibitor of hydrolase” mean in context of the present invention in particular a compound capable of fully or partially preventing or reducing the physiologic activity of a hydrolase. The terms “antagonist” or “inhibitor” are used interchangeably herein. It is envisaged herein that the “hydrolase inhibitor” is a selective inhibitor of a hydrolase.

In the context of the present invention said inhibitor may, therefore, prevent, reduce, inhibit or inactivate the physiological activity of a hydrolase e.g. upon binding of said compound/substance (i.e. antagonist/inhibitor) to said hydrolase. As used herein, the term “antagonist” also encompasses competitive antagonists, (reversible) non-competitive antagonists or irreversible antagonist, as described, inter alia, in Mutschler, “Arzneimittelwirkungen” (1986), Wissenschaftliche Verlagsgesellschaft mbH, Stuttgart, Germany. Such an inhibition can be measured by determining substrate turnover.

In sum, the herein described hydrolase antagonist/inhibitor will, accordingly, lead to a decrease or reduction of a hydrolase activity.

It is envisaged and preferred herein that the antagonist of a hydrolase targets the hydrolase, specifically targets the active site of a hydrolase (e.g. the catalytic triad). The term “targeting” refers in this context to the (specific) binding to a hydrolase (and here in particular to the active site of a hydrolase) and/or the inhibition of the activity of a hydrolase, in particular the inhibition of the hydrolytic activity.

The antagonist(s) may be (a) small molecule drug(s), or (a) (small) binding molecule.

Inhibitors to be used herein can be (a) small molecule drug(s). The terms “small molecule drug” and “small molecule compound” are used interchangeably herein. (A) small molecule drug(s) to be used herein as inhibitor of a hydrolase can refer to an (organic) low molecular weight (<900 Daltons) compound. Small molecules can help to regulate a biological process and have usually a size in the order of 10-9 m. Antagonists to be used herein, like small molecules (drugs), can, for example, be identified by screening compound libraries, for example Enamine, Chembridge or Prestwick chemical libraries.

The inhibitor is preferably a selective inhibitor of the hydrolase.

Selectivity expresses the biologic fact that at a given compound concentration enzymes (or proteins) are affected to different degrees. In the case of enzymes selective inhibition can be defined as preferred inhibition by a compound at a given concentration. Or in other words, an enzyme (or protein) is selectively inhibited over another enzyme (or protein) when there is a concentration, which results in inhibition of the first enzyme (or protein) whereas the second enzyme (or protein) is not, or not substantially, affected. To compare compound effects on different enzymes it is crucial to employ similar assay formats, such as the fluorescence energy transfer (FRET) assay, Plus assay, Histone Methyltransferase (HMT) assays, thermoshift assays, biological readouts (of reporter proteins/enzymes, such as Cxcl1/CXCL8), enzyme-linked immunosorbent assay (ELISA), or chemical proteomics. For example, commercially available test kits, like the ELISA kit scan be employed.

The inhibitors to be used herein are preferably selective for a hydrolase, i.e. the compounds selectively inhibit a hydrolase. In other words, the hydrolase inhibitors/antagonists are preferably selective hydrolase inhibitors/antagonists.

The term “selective hydrolase inhibitor(s)” as used herein refers to (a) hydrolase inhibitor(s) as defined herein (in particular (a) small molecule drug(s)) that inhibit(s) or display(s) antagonism towards a hydrolase without displaying substantial inhibition or antagonism towards another protein or enzyme, in particular another enzyme (i.e. in particular another protein or enzyme that is not a hydrolase).

Accordingly, a hydrolase inhibitor that is selective for a hydrolase exhibits an hydrolase selectivity of greater than about 2-fold, 3-fold, 4-fold, 5-fold, 6-fold, 7-fold, 8-fold, 9-fold, 10-fold, 20-fold, 50-fold or greater than about 100-fold with respect to inhibition or antagonism of another protein or enzyme (i.e. in particular another protein or enzyme that is not a hydrolase). A hydrolase inhibitor that is selective for a hydrolase may exhibit a hydrolase selectivity of greater than about 10.000-fold with respect to the protein of interest in the composition comprising a protein.

For example, pan-hydrolase inhibitors (i.e. compounds that broadly inhibit substantially any hydrolase) (e.g. Orlistat) may be used in context of this invention. Pan-hydrolase inhibitors can be selective hydrolase inhibitors as defined herein. For example, Orlistat binds with high preference to a hydrolase, specifically a lipase, compared to other proteins (i.e. in particular compared to non-hydrolases, specifically non-lipases), but Orlistat can react with/inhibit different lipases.

The term “selective hydrolase inhibitor(s)” does not necessarily imply that the inhibitor is highly specific, as it can react with/inhibit different hydrolases (e.g. different lipases). A highly specific inhibitor would only react with a single enzyme (a single hydrolase) while discriminating all other proteins. Also envisaged herein is the use of a (highly) specific hydrolase inhibitor (i.e. an inhibitor only reacting with/inhibiting a single enzyme (a single hydrolase) while discriminating all other proteins). It is envisaged herein that the (highly) specific hydrolase inhibitor as defined herein can be a selective hydrolase inhibitor as defined herein and vice versa.

Furthermore, hydrolase inhibitors/antagonists are preferably potent hydrolase inhibitors/antagonists.

“Potency for a hydrolase” can also be determined or defined by IC50 values. For example, the IC50 value of hydrolase inhibitors in relation to a hydrolase is low, preferably below 0.2 μM, more preferably, below 0.15 μM, 0.14 μM, 0.13 μM, 0.12 μM or even lower. More preferably, the IC50 value is below 0.1 μM, 0.095 μM, 0.090 μM, 0.085 μM, 0.080 μM, 0.075 μM, 0.070 μM, 0.065 μM, 0.060 μM, 0.055 μM, 0.050 μM, 0.045 μM, 0.040 μM, 0.035 μM, 0.030 μM, or even below 0.025 μM, wherein the lower values are preferred over the higher values. Even more preferably, the IC50 value is below 0.024 μM, 0.023 μM, 0.022 μM, 0.021 μM, 0.020 μM, 0.019 μM, 0.018 μM, 0.017 μM, 0.016 μM, 0.015 μM, 0.014 μM, 0.013 μM, 0.012 μM, or 0.011 μM. The IC50 value may even be lower, for example, below 0.010 μM, 0.009 μM, 0.008 μM, 0.007 μM, 0.006 μM, or 0.005 μM. Generally, the lower values are preferred herein over the higher values.

Potent hydrolase inhibitors in accordance with the present invention can, in the alternative, or in addition to the IC50 value in relation to a hydrolase, be defined by IC50 value in relation to another protein or enzyme.

For example, the IC50 value of potent hydrolase inhibitors in relation to another protein or enzyme is high, preferably higher than 0.001 μM, 0.002 μM, 0.003 μM, 0.004 μM, 0.005 μM, 0.006 μM, 0.007 μM, 0.008 μM, 0.009 μM, or 0.010 μM. More preferably, the IC50 value is higher than 0.011 μM, 0.012 μM, 0.013 μM, 0.014 μM, 0.015 μM, 0.016 μM, 0.017 μM, 0.018 μM, 0.019 μM, 0.020 μM, 0.021 μM, 0.022 μM, 0.023 μM, or 0.024 μM. Even more preferably, the IC50 value is higher than 0.025 μM, 0.030 μM, 0.035 μM, 0.040 μM, 0.045 μM, 0.050 μM, 0.055 μM, 0.060 μM, 0.065 μM, 0.070 μM, 0.075 μM, 0.080 μM, 0.085 μM, 0.090 μM, 0.095 μM, 0.1 μM, or even higher, wherein the higher values are preferred over the lower values. Even more preferably, the IC50 value is higher than 0.12 JIM, 0.13 μM, 0.14 JIM, 0.15 μM, 0.2 μM or even higher.

It is preferred herein that the ratio of IC50 values of potent hydrolase inhibitors towards a hydrolase in relation to IC50 values of another protein or enzyme towards a hydrolase, preferably determined according to the same assay, is about 1:10 or lower. A ratio of 1:10 or lower also indicates potency of the inhibitor for a hydrolase. More preferred is a ratio of 1:10, 1:20, 1:30, 1:40, 1:50, 1:60, 1:70, 1:80, 1:90 or 1:100 or even lower.

The term “hydrolase” is used herein in the broadest sense and refers to a molecule/compound with hydrolytic activity. Generally, a “hydrolase” is thus an enzyme that hydrolyzes a chemical bond. For example, a “hydrolase” is a carboxylic derived ester cleaving enzyme which has as final product the derived free acid. Alternatively, a hydrolase is an enzyme exhibiting hydrolase activity, i.e. breaking covalent bond by using a water molecule. Non-limiting examples of a “hydrolase” are a lipase, an esterase, a thioesterase, a phospholipase or a ceramidase.

“Hydrolytic activity” as used herein as known in the art and refers to the ability to hydrolyze chemical bonds. Hydrolysis refers to the cleavage of a chemical bond in which a water molecule acts as a nucleophile. Preferably, the molecule/compound with hydrolytic activity is a protein or a polypeptide with hydrolytic activity. Accordingly, the protein or polypeptide with hydrolytic activity is an enzyme with hydrolytic activity. Thus, the hydrolase preferably is an enzyme with hydrolytic activity. The hydrolase may be an esterase or an amidase.

The term “esterase” is well known in the art and refers in context of this invention to an enzyme that catalyzes the hydrolysis of an ester bond to create an acid and an alcohol. In other words, an esterase may act on (an) ester bond. Esterases are a diverse category of enzymes, including acetyl esterases, phosphatases, nucleases, thioesterases, and carboxylic ester hydrolases. Preferably, the hydrolase in context of the present invention is a thioesterase or a carboxylic ester hydrolase. A thioesterase uses a water molecule to hydrolyze a thioester into a thiol and an acid. A carboxylic ester hydrolase uses a water molecule to hydrolyze a carboxylic ester into an alcohol and a carboxylate. Most preferably, a hydrolase/esterase (such as a carboxylic ester hydrolase) is a lipase. A lipase catalyzes the hydrolysis of fatty acid esters/lipids, including triglycerides, fats and oils into fatty acids and an alcohol head group.

The term “amidase” is well known in the art and refers in context of this invention to an enzyme that catalyzes the hydrolysis of an amide bond. In other words, an amidase may act on (an) amide bond.

Preferably, (a) hydrolase(s) according to the invention is Lipoprotein Lipase (Accession number: G3H6V7), Palmitoyl Proteinthioesterase 1 (Accession number: G3HN89), Acid Ceramidase (Accession number: G3GZB2), the C-terminal domain of Fatty Acid synthase (Accession number: G3GXD7), Putative Phospholipase b-like 2 (Accession number: G3I6T1), Lysosomal Acid Lipase (Accession number: G3HQY6) and/or Lysosomal Phospholipase. More preferably, (a) hydrolase(s) according to the invention is Lipoprotein Lipase, Putative Phospholipase b-like 2, and/or Lysosomal Phospholipase. This is because of their documented hydrolytic activity towards surfactants, such as polysorbate, which is attributed to the formation of (visible) particles in compositions and/or decreased stability of compositions. Most preferably, the hydrolase(s) according to the invention is Lysosomal Phospholipase, specifically lysosomal phospholipase A2 (LPLA2) (Accession number: G3HKV9).

That means that the hydrolase(s) that is/are removed from (or whose content is reduced in) the starting composition comprising the protein may be selected from the group consisting of Lipoprotein Lipase, Pahnitoyl Proteinthioesterase, Acid Ceramidase, Fatty Acid synthase (in particular the C-terminal domain thereof), Putative Phospholipase b-like 2, Lysosomal Acid Lipase and Lysosomal Phospholipase. Accordingly, the hydrolase(s) that is/are bound to the hydrolase inhibitor may be selected from the group consisting of Lipoprotein Lipase, Palmitoyl Proteinthioesterase, Acid Ceramidase, Fatty Acid synthase, Putative Phospholipase b-like 2, Lysosomal Acid Lipase and Lysosomal Phospholipase.

The term “immobilized on a solid carrier” means that the hydrolase inhibitor is attached to a solid carrier (the terms “solid carrier” and “solid phase” are used interchangeably herein). In other words, the hydrolase inhibitor is bound to a solid carrier. Non-limiting examples of solid carriers are provided herein below. The bond(s) between the hydrolase inhibitor and the solid carrier may be a covalent bond and/or a non-covalent bond. Preferably, the bond is a covalent bond. Non-limiting examples of how the hydrolase inhibitor may be immobilized on a solid carrier are described herein below and in the appended examples.

“Recovering a composition comprising the protein” means that the composition comprising the protein is collected after it was contacted with the hydrolase inhibitor. Accordingly, the “starting composition comprising a protein” is a composition comprising a protein before that composition was contacted with the hydrolase inhibitor. The “composition comprising the protein” is a composition comprising a protein after that composition (here the “starting composition”) was contacted with the hydrolase inhibitor (and the “composition comprising the protein” is then the recovered composition comprising the protein).

For example, the steps (i) and (ii) can be carried out as follows:

The method can comprise a first step (step (i)) comprising providing a solid carrier (or solid phase), the carrier comprising a hydrolase inhibitor immobilized thereto, and contacting a starting composition with the hydrolase inhibitor (or with the solid carrier to which the inhibitor is immobilized). The hydrolase then binds to the hydrolase inhibitor while the remaining proteins (or at least a major part thereof), particularly the protein of interest, do not bind to the inhibitor and can be collected (e.g. from the flow-through) (or bind to a less extent than the hydrolase).

The method can further comprise—for recovering a composition comprising the protein ((step (ii))—performing several wash steps, wherein at least one wash step is performed using a wash buffer capable of detaching at least part of the protein of interest, that might have bound to the inhibitor/carrier, from the solid carrier and/or inhibitor, while retaining (at least part of) the impurity (host cell protein, such as hydrolase) bound to the solid carrier and/or inhibitor. Optionally, the method can comprise obtaining the protein of interest using an elution buffer capable of detaching (at least part of) said protein from the solid carrier and/or inhibitor, in case the protein might have bound to the inhibitor/carrier, while retaining (at least part of) the impurity (host cell protein, such as hydrolase) bound to the solid carrier and/or inhibitor.

As discussed above, it is envisaged herein that the composition comprising the protein has reduced hydrolytic activity compared to the starting composition comprising the protein. Accordingly, in one aspect, the invention relates to a method comprising the steps of
(i) contacting a starting composition comprising a protein with a hydrolase inhibitor, wherein the hydrolase inhibitor is immobilized on a solid carrier;
(ii) recovering a composition comprising the protein,

wherein the composition comprising the protein has reduced hydrolytic activity compared to the starting composition.

Accordingly, the invention provides in one aspect a method for preparing or for obtaining a composition comprising a protein comprising the steps of
(i) contacting a starting composition comprising a protein with a hydrolase inhibitor, wherein the hydrolase inhibitor is immobilized on a solid carrier;
(ii) recovering a composition comprising the protein,

wherein the composition comprising the protein has reduced hydrolytic activity compared to the starting composition.

The composition comprising the protein (the recovered composition) may have a reduced hydrolytic activity compared to the starting composition comprising a protein because (a) hydrolase(s) has/have been removed from the starting composition comprising the protein and/or the content of (a) hydrolase(s) has been reduced in the starting composition.

A reduction of hydrolytic activity compared to the starting composition comprising a protein implies of course that the starting composition has hydrolytic activity. Similarly, a reduction of the content of (a) hydrolase(s) compared to the starting composition comprising a protein implies of course that the starting composition comprises (a) hydrolase(s).

Accordingly, in the method as described herein the starting composition comprising a protein may have hydrolytic activity and/or comprise one or more hydrolase(s).

Thus, the method of the invention can comprise a step of determining the hydrolytic activity of the starting composition comprising a protein and/or determining the hydrolytic activity of the composition comprising the protein (the recovered composition). When both steps are carried out (i.e. when both the hydrolytic activity of the starting composition comprising a protein and of the composition comprising the protein (the recovered composition) is determined, it can readily be determined whether the composition comprising the protein (the recovered composition) has a reduced hydrolytic activity compared to the starting composition.

In one aspect, when (only) the step of determining the hydrolytic activity of the starting composition comprising a protein is carried out and when it is determined that the starting composition does not have hydrolytic activity, step (i) (and step (ii)) of the claim is not carried out.

In one aspect, when (only) the step of determining the hydrolytic activity of the starting composition comprising a protein is carried out and when it is determined that the starting composition does have hydrolytic activity, step (i) (and step (ii)) of the claim is carried out.

The same explanations apply mutatis mutandis to the aspect described above that the starting composition comprising a protein comprises one or more hydrolase(s).

An indication of the presence of hydrolases is (an increased) break down of substrate (e.g. intact fatty acid ester, e.g. polysorbate) and/or (simultaneous) increase in free fatty acid content in a protein composition/formulation, particularly if compared to a composition/formulation that has been confirmed to be hydrolase-free. If such a breakdown is determined, the need for performing this method is indicated in the first place. However, the method can be used as additional clearance step for hard-to-remove-HCPs in general.

The skilled person is well aware how hydrolytic activity can be measured, determined or quantified.

Non-limiting examples for measuring, determining or quantifying hydrolytic activity are an assay to detect lipolytic activity or, in other words, a lipase activity assay (e.g. as described by Jahn et al.), herein also termed LEAP (lipase enzymatic assay for polysorbases) assay (Jahn (2020) Pharm. Res. 37: 118), and/or a fatty acid by mass spectrometry (FAMS) assay (Honemann (2019) J. Chromatogr. B 1116: 1-8; Cheng (2019) J. Pharm. Sci. 108: 2880-2886). A non-limiting example for assays that allow measuring, determining or quantifying hydrolytic activity is a lipase activity assay (or in other words an assay to detect lipolytic activity) measuring the conversion of 4-methylumbelliferone caprylate (4-MUCA) to 4-methylumbelliferone (4-MU). An assay using 4-MUCA as a substrate may be performed as described in the following.

10 μL of the composition comprising the protein in which hydrolytic activity is to be determined may be mixed with 80 μL of reaction buffer (150 mM Tris-HCl pH 8.0, 0.25% (w/v) Triton X-100 and 0.125% (w/v) Gum Arabic) and 10 μL 4-MUCA substrate (1 mM in DMSO). The reactions may be set up in 96-well half-area polystyrene plates (black with lid and clear flat bottom, Corning Incorporated) and the increase of fluorescent signal (excitation at 355 nm, emission at 460 nm) may be monitored every 10 min by incubating the reaction plate for two hours at 37° C. for example in an Infinite 200Pro plate reader (Tecan Life Sciences) to derive the 4-MU production rate. The 4-MU production rate of the composition comprising the protein may be compared with the starting composition comprising a protein.

The following paragraph describes an exemplary sample preparation and analytical procedure for performing a FAMS assay. The FAMS assay measures the lipase activity by quantifying the accumulation of free fatty acids through the hydrolysis of Polysorbate 20.

All samples (antibody solutions and buffer controls) may be supplemented with a stock solution of 1% (w/v) super-refined Polysorbate 20 (Croda Health Care) and 0.25 M L-Methionine (Sigma Aldrich, Art. No. M5308) to obtain a final concentration per sample of 0.04% (w/v) super-refined Polysorbate 20 and 0.01 M L-Methionine. For each spiked sample, aliquots of 190 μL may be transferred in five capped glass vials (termed t0, t1, t2, t3, t4), which may be used for sample incubation. The glass vials t0 may be frozen immediately at −70° C. after their preparation until analysis. The glass vials t1, t2, t3 and t4 may be incubated at 25° C. upright and protected from light in an incubator. Incubation of the glass vials may be stopped one at a time over a time period between the next five day and 14 days the glass vials may be subsequently frozen at −70° C. until analysis.

For analysis, frozen samples may be brought to ambient temperature for 1 hour. Stock solutions of stable isotope labelled fatty acids may be prepared by dissolving 50 mg of the respective fatty acid, lauric acid ²d₂₃ (Sigma Aldrich, Cat. No. 451401) and myristic acid ¹³C₁₄ (Sigma Aldrich, Cat. No. 605689) in 50 mL 80% acetone/20% methanol methanol to obtain the precipitation reagent with a final concentration of 1 μg/mL for each investigated fatty acid. 50 μL of the sample solution may be added to 200 μL of the precipitation reagent in a reaction tube, mixed by vortexing and kept by room temperature for 1 h to allow the protein to precipitate. The precipitate may be spun down by centrifugation at 20° C. with 15,000×g for 15 min. 100 μL of the supernatant may be transferred in a fresh reaction tube and mixed with 100 μL of Mobile phase A (20 mM ammonium acetate). After spinning down at 20° C. with 15,000×g for 15 min, 50-100 μL of the solution may be transferred into LC-MS vials (300 μl Fixed Insert Vial (Clear, Screw Top), Thermo Scientific, Cat. No. 03-FISV).

Separation of free fatty acids (FFAs) may be achieved on an ACQUITY UPLC H-Class System (Waters Corporation, Milford, MA, US) equipped with a temperature-controllable autosampler and column compartment by using a Jupiter® C4 RP column (300 Å, 2×50 mm, 5 μm) (Phenomenex, Cat. No. 00B-4167-B0). Mobile Phase A may be 20 mM ammonium acetate (Sigma Aldrich, Cat. No. 73594) and Mobile Phase B may be 100% methanol (Merck, Cat. No. 1.06007.2500), which may be run for 4 min at a flow rate of 0.4 mL/min by applying the following gradient: Initial conditions: 70% Mobile Phase B, 0.5 min-3.4 min: gradient may be changed linearly from 70% to 85% Mobile Phase B; 3.5-4.0 min: 70% Mobile Phase B. The autosampler may be maintained at 20° C. and the column compartment at 60° C. The injection volume may be set to 8 μL. Detection may be performed on a connected QDa Performance mass spectrometer (Waters) equipped with an external backing pump in negative ion mode. The MS settings may be as follows, cone voltage 15 V, source temperature 120° C., capillary voltage 800 V, probe temperature 600° C., mass range 50-1000 m/z and sampling frequency 2 Hz. All samples may be analyzed in triplicates.

Data evaluation may be performed with TargetLynx as part of the MassLynx Software Version 4.1 (SCN781) (Waters Corporation, Milford, MA, US). The content of lauric and myristic may be determined by comparing the peak area of the fatty acid with the respective internal labelled standard by using the following formula:

$$Conc . ( FFA ) = \frac{\sum Peak areas of FFA}{\sum Peak areas of Internal standard} * 4 * 1 μ g / mL$$

where ΣPeak areas of FFA and ΣPeak areas of Internal standard refer to the sum of the monoisotopic peak and the isotopic peaks at +1/+2 or −1/−2, respectively.

For determining the fatty acid release rate, the free fatty acid concentration for each sample may be plotted against the incubation time and the degradation rate may be extracted from the slope of the linear regression.

The hydrolytic activity of the composition comprising the protein may be reduced compared to the starting composition comprising the protein by at least 5%, at least 10%, at least 20%, at least 30%, at least 40%, at least 50%, at least 60%, at least 70%, at least 80%, at least 90%, at least 95% or at least 99%, for example from 20 up to 80%, preferably from 50 up to 80%. Vice versa, the hydrolytic activity of the composition comprising the protein may be reduced compared to the starting composition comprising the protein by up to 5%, up to 10%, up to 20%, up to 30%, up to 40%, up to 50%, up to 60%, up to 70%, up to 80%, up to 90%, up to 95% or up to 99%, preferably by up to 20%, more preferably up to 50%, even more preferably by up to 80%. If a composition is subject to one or more purifications by the herein provided method, as explained further below, the % reduction as defined herein refers preferably to the (initial, first) starting composition as a reference point, i.e. to a starting composition that has not been contacted with a hydrolase inhibitor as described herein.

Of course, it is also envisaged that the hydrolytic activity in the composition comprising the protein is reduced to a level below the detection limit of the employed assay (in the latter case hydrolytic activity and/or hydrolase(s) are considered “removed”, or more precisely “entirely removed” from the starting composition herein).

The invention also relates to a method for preparing or for obtaining a composition comprising a protein wherein the composition comprising the protein has reduced hydrolytic activity compared to the starting composition, wherein the hydrolytic activity may be determined for example by one of the assays described herein (e.g. lipase activity assay (e.g. as described by Jahn et al.), herein termed LEAP (lipase enzymatic assay for polysorbases) assay (Jahn (2020) Pharm. Res. 37: 118), and/or a fatty acid by mass spectrometry (FAMS) assay).

Likewise, the composition may comprise a reduced content of host cell protein (HCP), such as a hydrolase, compared to the starting composition. For example, the HCP content may be reduced compared to the starting composition by 5%, 10%, 20%, 30%, 40%, 50%, 60%, 70%, 80%, 90%, 95% or 99%. For example, the composition comprises preferably less than 100 ppm HCP, more preferably less than 10 ppm, more preferably less than 1 ppm, more preferably less than 0.1 ppm, most preferably less than 0.01 ppm.

It will be appreciated that the method described herein can also be employed when the starting composition comprising the protein does not exhibit a detectable hydrolytic activity in the employed assays for measuring or quantifying hydrolytic activity. Without necessarily being bound by scientific theory it is envisaged that also hydrolytic activity that is below the detection limit of the employed assay in the composition comprising the protein may have a negative effect on the composition comprising the protein. Said negative effect may become apparent only during prolonged storage of the composition comprising the protein. Said negative effect may be the degradation of a surfactant, e.g. fatty acid ester, an ester or a thioester and is described herein in more detail.

It is also envisaged herein that the produced/recovered composition comprising the protein is essentially free of hydrolytic activity.

Accordingly, the invention relates to a method comprising the steps of
(i) contacting a starting composition comprising a protein with a hydrolase inhibitor, wherein the hydrolase inhibitor is immobilized on a solid carrier;
(ii) recovering a composition comprising the protein,

wherein the composition comprising the protein is essentially free of hydrolytic activity.

Furthermore, the invention provides a method for preparing or for obtaining a composition comprising a protein comprising the steps of
(i) contacting a starting composition comprising a protein with a hydrolase inhibitor, wherein the hydrolase inhibitor is immobilized on a solid carrier;
(ii) recovering a composition comprising the protein,

wherein the composition comprising the protein is essentially free of hydrolytic activity.

“Essentially free of hydrolytic activity” as used herein can mean that the hydrolytic activity in the composition comprising the protein is near or below the detection limit of the assay used to measure or quantify the hydrolytic activity. “Essentially free of hydrolytic activity” as used herein can also mean that the hydrolytic activity of the composition comprising the protein is reduced compared to the starting composition comprising the protein by 80%, 90%, 95% or 99%.

“Essentially free of hydrolytic activity” as used herein can also mean that less than 10% of (an) ester(s) in the composition comprising the protein is/are degraded when the composition comprising the protein is stored, e.g. for 24 months at 4° C. to 8° C., e.g. for at least 6 months, at least 12 months, at least 18 months, at least 24 months, at least 36 months or at least 60 months, and preferably under standard long-term conditions as described herein. The composition may comprise protein concentrations as described herein, buffer systems as described herein, additives as described herein and surfactants as described herein (e.g. Polysorbate 20 or Polysorbate 80) ranging from 0.01% (w/v) and 2% (w/v).

The degradation may be monitored by determining the remaining surfactant content by mixed-mode HPLC coupled to ELSD/CAD detection (Lippold (2017) J. Pharm. Biomed. Anal. 132:24-34) or a so-called Fluorescence Micelle Assay (FMA) (Lippold (2017) J. Pharm. Biomed. Anal. 132:24-34). Also, a FAMS assay may be used to directly monitor the main degradation products (e.g., lauric and myristic acid in the case of PS20, oleic acid in the case of PS80) (Honemann (2019) J. Chromatogr. B 1116:1-8; Cheng (2019) J. Pharm. Sci. 108:2880-2886).

The use and adaption of the above mentioned assays is well within the skills of the relevant artisan.

It will be appreciated that the method described herein may be used for reducing hydrolytic activity and/or for reducing the content of hydrolase(s) in the starting composition comprising a protein.

Therefore, the invention also relates in one aspect to a method comprising the steps of
(i) contacting a starting composition comprising a protein with a hydrolase inhibitor, wherein the hydrolase inhibitor is immobilized on a solid carrier;
(ii) recovering a composition comprising the protein,

wherein the method is for reducing the hydrolytic activity in the starting composition.

Additionally, the invention provides in one aspect a method for preparing or for obtaining a composition comprising a protein comprising the steps of
(i) contacting a starting composition comprising a protein with a hydrolase inhibitor, wherein the hydrolase inhibitor is immobilized on a solid carrier;
(ii) recovering a composition comprising the protein

wherein the method is for reducing the hydrolytic activity in the starting composition.

It has been described in detail hereinabove how the hydrolytic activity and/or the content of hydrolase(s) can be reduced and how a corresponding reduction can be determined.

Thus, the invention also provides in one aspect for a method to remove a hydrolase from a protein composition and/or to reduce the content of hydrolase(s) in a protein composition.

Generally, the terms “protein composition” and “composition comprising a/the protein” are used interchangeably herein.

In other words, the invention relates in one aspect to a method wherein the composition comprising the protein is prepared by removing a hydrolase from the starting composition comprising a protein and/or by reducing the content of hydrolase(s) in the starting composition.

Accordingly, the invention relates in one aspect to a method wherein the composition comprising the protein is prepared by removing a hydrolase from the starting composition comprising the protein comprising the steps of
(i) contacting a starting composition comprising a protein with a hydrolase inhibitor, wherein the hydrolase inhibitor is immobilized on a solid carrier;
(ii) recovering a composition comprising the protein.

Accordingly, in the method as described herein the starting composition comprising the protein may comprise one or more hydrolase(s).

The method as described herein may further comprise a step for adsorbing the hydrolase to the hydrolase inhibitor. Thus, the invention relates in one aspect to a method comprising the steps of
(i) contacting a starting composition comprising a protein with a hydrolase inhibitor, wherein the hydrolase inhibitor is immobilized on a solid carrier;
(ii) recovering a composition comprising the protein;

wherein said step (i) further comprises a step (a) adsorbing the hydrolase to the hydrolase inhibitor. “Adsorbing” is used herein in the broadest sense and means that the hydrolase associates with the hydrolase inhibitor. In other words, the hydrolase binds to the hydrolase inhibitor. The hydrolase and the hydrolase inhibitor may associate via a non-covalent bond or via a covalent bond. Examples for non-covalent bonds are hydrogen-bridges, ionic interactions and hydrophobic interactions. Preferably, the hydrolase and the hydrolase inhibitor associate via a covalent bond. The covalent bond may be an ester, a thioester or a phosphoester.

It is envisaged that the beta-Lactone moiety of Orlistat forms an ester bond with a serine of the hydrolase.

It is further envisaged that the composition comprising the protein (the recovered composition) may be a solution comprising the protein. Also, the starting composition comprising a protein may be a solution comprising a protein.

Said solution comprising a protein may be an aqueous solution.

Said solution comprising a protein may be a buffered solution. Accordingly, also said aqueous solution comprising the protein may be a buffered solution.

It is well known in the art which agents can be used as a buffer to buffer a solution. The term “buffer” is used herein as known in the art and refers to an agent that stabilizes the pH of a solution. A buffer generally comprises a weak acid and its conjugate base, or a weak base and its conjugate acid. The skilled person knows how an optimal pH for e.g. a certain protein can be determined and which buffer may be used to stabilize said optimal pH. Non-limiting examples for agents that can be used as buffer are Tris, Hepes, Pipes, Mops, acetate and phosphate.

As described above the method may comprise contacting a starting composition comprising a protein with a hydrolase inhibitor. Hydrolase inhibitors as well as non-limiting examples of hydrolase inhibitor are described herein. Additionally, the hydrolase inhibitor may be selected from the group consisting of orlistat or bis-enol-ester, phosphonates e.g. a-aminoalkylphosphonate or para-nitrophenyl phosphonate (e.g. Methyl 4-nitrophenyl undec-10-enylphosphonate) (Delorme (2014) Biochimie 107: 124-134) As already mentioned above the hydrolase inhibitor may be immobilized/attached to a solid carrier. The terms “solid carrier” and “solid phase” are used interchangeably herein.

The solid carrier may be a resin and in the form of a column, for example, a commercially available resin/column. The solid phase may for instance be a resin and is typically in the form of a column, which is as such commercially available. Use of a column in a method according to the invention is preferred as it is easily up-scalable. Since the hydrolase inhibitor binds the hydrolase covalently such a column may be for single use. Accordingly, it is not necessary to regenerate the column. It is envisaged that such a column comprising the hydrolase inhibitor immobilized to the solid carrier has a smaller volume than e.g. a column for hydrophobic interaction chromatography (HIC) used in a comparable process step (a HIC column typically has a volume of about 20 L). The volume of a column comprising the hydrolase inhibitor immobilized to the solid carrier may be between about 250 ml and up to about 5 l, e.g. may be between about 250 ml to about 5 L, about 250 ml to about 4 L, about 250 ml to about 3 L, about 250 ml to about 2 L, about 250 ml to about 1.5 L, about 250 ml to about 1 L or may be between about 500 ml to about 5 L, about 500 ml to about 4 L, about 500 ml to about 3 L, about 500 ml to about 2 L, about 500 ml to about 1.5 L, about 500 ml to about 1 L, e.g. may be about 500 ml, about 1 L, about 1.5 L, about 2 L, about 3 L, about 4 L or about 5 L. Preferably the volume of a column comprising the hydrolase inhibitor immobilized to the solid carrier is between about 500 ml to about 1.5 L, more preferably between about 500 ml to about 1 L. Most preferably, the volume of a column comprising the hydrolase inhibitor immobilized to the solid carrier is about 500 ml, about 600 ml, about 700 ml, about 800 ml, about 900 ml, about 1 L, about 1.1 L, about 1.2 L, about 1.3 L, about 1.4 L or about 1.5 L. Particularly preferred is a volume of a column comprising the hydrolase inhibitor immobilized to the solid carrier of about 500 ml or about 1 L.

It is, however, also possible that the solid carrier is in form of beads or in form of a membrane.

If, for example, the solid carrier is in form of beads comprising said inhibitor, the method can comprise performing the steps of contacting and recovery through centrifugation, decanting and dissolution. It is also possible to use magnetic beads and magnetic separation during the recovery step (which can include washing steps). A person skilled in the art is familiar with the different processes for affinity purification and can easily adapt such processes for use in a method according to the invention.

The solid carrier may be selected from the group consisting of sepharose, polystyrene, inorganic metal and/or metal-heteroatom particles (e.g. Fe₃O₄, SiO₂ or FeS particles), gold particles, silver particles and smart polymers. It is also possible that the solid carrier is in form of magnetic beads and magnetic separation can be employed when performing the method. The skilled person is familiar with the different processes generally used in context of affinity purification and can easily adapt such processes for use in a method according to the invention.

Although the skilled person is well aware how a hydrolase inhibitor can be immobilized on a solid carrier, non-limiting examples are provided in the following. Generally, the hydrolase inhibitor can be immobilized on a solid carrier via the linkage of functional groups and/or biomolecule/ligand binding.

The following explanations, as well as the explanations herein above in relation to a hydrolase inhibitor, also apply mutatis mutandis to a method for preparing a hydrolase inhibitor immobilized on a solid carrier, e.g. for preparing a device (such as a column) comprising a hydrolase inhibitor immobilized on a solid carrier. Such a method for preparing a hydrolase inhibitor immobilized on a solid carrier (or for preparing a device (such as a column) comprising a hydrolase inhibitor immobilized on a solid carrier) is explicitly considered an aspect of the present invention. The hydrolase inhibitor(s) immobilized on a solid carrier and/or a device (such as a column) comprising a hydrolase inhibitor immobilized on a solid carrier are also an aspect of the present invention. Vice versa, the explanations and definitions herein in relation to the preparation of a device (such as a column) comprising a hydrolase inhibitor immobilized on a solid carrier and to such a device as such also apply mutatis mutandis to a hydrolase inhibitor immobilized on a solid carrier and/or a method for preparing same. It is understood that when a hydrolase inhibitor should be immobilized on a solid carrier in accordance with the invention a derivative of the hydrolase inhibitor as explained herein is to be used.

The hydrolase inhibitor may be immobilized on a solid carrier via the reaction of an azido group and an alkyne group. Said reaction may be an Azide-alkyne Huisgen cycloaddition. The alkyne group may be attached to the hydrolase inhibitor while the azido group may be attached to the solid carrier. However, it is also possible that the alkyne group may be attached to the solid carrier while the azido group may be attached to the hydrolase inhibitor.

Accordingly, the hydrolase inhibitor may be immobilized to azide modified magnetic particles (Polystyrene based azide beads; CLK-1036-1, Jena-Bioscience). The terms azido group and azide group are used interchangeably herein.

Furthermore, the hydrolase inhibitor may be immobilized on a solid carrier via the binding of a streptavidin group to a biotin group. The streptavidin group may be attached to the hydrolase inhibitor while the biotin group may be attached to the solid carrier. Preferably, the streptavidin group may be attached to the solid carrier while the biotin group is attached to the hydrolase inhibitor.

Furthermore, the hydrolase inhibitor may be immobilized on a solid carrier via the reaction of an amino group and an N-Hydroxysuccinimid-activated ester. The amino group may be attached to the hydrolase inhibitor while the N-Hydroxysuccinimid-activated ester is attached to the solid carrier. However, it is also possible that the amino group is attached to the solid carrier while the N-Hydroxysuccinimid-activated ester is attached to the hydrolase inhibitor.

Although clear for the skilled person it is noted that the hydrolase inhibitor or the solid carrier may be separated from the reactive groups described above via a linker. Linkers are described herein below.

Although clear for the skilled person it is noted that the means and methods to immobilize the hydrolase inhibitor on the solid carrier as described above can be combined. For example, it is envisaged that an agent comprising an azido group and a biotin group separated by a linker is first coupled via click chemistry to the hydrolase inhibitor comprising an alkyne group. Subsequently, the hydrolase inhibitor coupled to said agent is immobilized on a solid carrier comprising a streptavidin group. The skilled person is well aware which linker may be suitable. Such linker may facilitate complex formation and/or establish a defined distance between the hydrolase inhibitor and the solid carrier. A linker can in principle be any chemical linkage suitable to covalently link the hydrolase inhibitor on the solid carrier in the desired distance. The skilled person can select the linker according to the needs with respect to resistance of the bond to the environment and conditions of the intended use. A linker may, for example, comprise a PEG or a biopolymer such as DNA/LNA, Poly Sugars (e.g. Chitosan or derivatives), maleimide alkane linkers, chemically cleavable linkers (e.g. hydrazine/disulfide linker) or enzymatically cleavable linkers (e.g. Val-Cit-PABC linkers). A PEG linker may be PEG3 or PEG4. In a preferred embodiment the agent/linker comprising an azido group and a biotin group separated by a PEG linker is azo biotin-PEG3-azide (Sigma-Aldrich; Order Number: 900891; CAS 1339202-33-3) also referred to azo-biotin-azide herein because it is distributed under the name azo-biotin-azide. In a preferred embodiment the hydrolase inhibitor coupled to said agent is immobilized on Streptavidin Mag Sepharose beads (GE Healthcare; Order Number: 11791456). In another preferred embodiment the agent comprising an azido group and a biotin group separated by a PEG linker is biotin-PEG4-azide (BroadPharm; Catalog Number: BP-22119; CAS 1309649-57-7). In a preferred embodiment the hydrolase inhibitor coupled to said agent is immobilized on Streptavidin Sepharose® High Performance beads (GE Healthcare; Order Number: 17511301).

In addition, it is envisaged herein that an agent comprising an azido group and an amino group separated by a linker is first coupled to a solid carrier comprising an NHS-activated ester. Subsequently, the hydrolase inhibitor comprising an alkyne group is coupled to said agent immobilized on the solid carrier. A description for linker is described above. In a preferred embodiment the agent comprising an azido group and an amino group separated by a linker is Azido-PEG4-Amine (Broadpharm, BP-21615; CAS 951671-92-4). In a preferred embodiment the hydrolase inhibitor coupled to said agent is immobilized on NHS-activated Sepharose® 4 Fast Flow beads (GE Healthcare; Order Number: 17090601).

The following relates to inhibitors that can be used in accordance with the invention.

Preferably, the hydrolase inhibitor (in the form before being attached on the solid phase) is selected from compounds comprising or consisting of (e.g. having a structure) selected from:

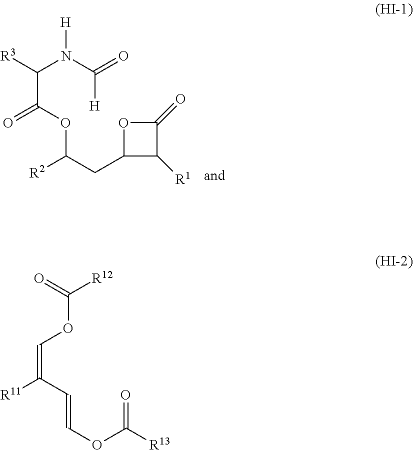

wherein in (HI-1)
R¹, R² and R³ are each independently selected from C₂₋₂₄-alkyl which may be linear or branched, optionally wherein at least one terminal —CH₂—CH₃ group in R¹, R² or R³ is replaced by a —C≡CH group,

wherein in (HI-2)
R¹¹, R¹² and R¹³ are each independently selected from C₂₋₂₄-alkyl which may be linear or branched, optionally and wherein at least one terminal —CH₂—CH₃ group in R¹¹, R¹² and R¹³ is replaced by a —C≡CH group.

The hydrolase inhibitor, as present on the solid carrier, is preferably selected from compounds comprising, or consisting of, a structure selected from:

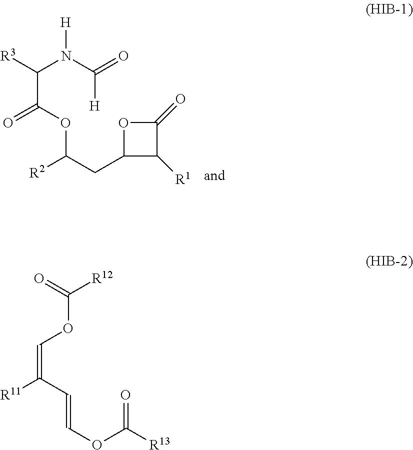

wherein in (HIB-1)
R¹, R² and R³ are each independently selected from C₂₋₂₄-alkyl which may be linear or branched, wherein one terminal —CH₃ group in R¹, R² or R³ (preferably in R²) is replaced by a —CH₂—* group, in which -* indicates the position where the hydrolase inhibitor is linked to the solid carrier,

wherein in (HIB-2)
R¹¹, R¹² and R¹³ are each independently selected from C₂₋₂₄-alkyl which may be linear or branched, and wherein one terminal —CH₃ group in R¹¹, R¹² or R¹³ (preferably in R¹¹) is replaced by a —CH₂—* group, in which -* indicates the position where the hydrolase inhibitor is linked to the solid carrier.

The number of carbon atoms in R¹, R² and R¹¹ can independently be selected from 2 to 15, preferably 3 to 14, more preferably 4 to 12, even more preferably from 5 to 11.

The number of carbon atoms in R¹² and R¹³ can independently be selected from 5 to 24, preferably 8 to 18, more preferably 10 to 16, even more preferably from 10 to 12.

The number of carbon atoms in R³ can be selected from 2 to 10, preferably 2 to 8, more preferably 3 to 6, even more preferably from 3 to 5.

The alkyl groups of R¹ and R² are preferably linear.

The alkyl groups of R¹¹ R¹² and R¹³ are preferably linear.

The alkyl group of R³ is preferably branched.

Preferably formula (HIB-1) has the following structure (HIB-1a):

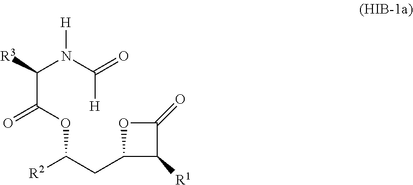

wherein R¹, R² and R³ are as defined above.

In the above examples of (HI-1) and (HI-2), (in the form of the inhibitor before being attached to the solid phase) as well as in any more specific examples thereof, functional groups other than the —C≡CH group may be included such as hydroxyl groups, carboxylate groups, ketones, aldehydes, isocyanates, epoxides, hydroxyl groups, carboxylate groups, amines, other alkynes, azides or biopolymers (e.g. streptavidin and/or biotin). It is to be understood that the hydrolase inhibitor, as present on the solid carrier, will be bound to the solid carrier via the linker groups resulting from the functional groups, e.g. via a 1,2,3-triazole in the case where the inhibitor (in the form before being attached to the solid phase) had an alkynyl groups and was bound (directly or indirectly) to the solid phase by a reaction with an azide, which are present on the solid carrier and the hydrolase inhibitor before the hydrolase inhibitor is bound to the solid phase.

Preferably, the hydrolase inhibitor (in the form of the inhibitor before being attached to the solid phase) contains an alkyne group and is selected from compounds having a structure selected from:

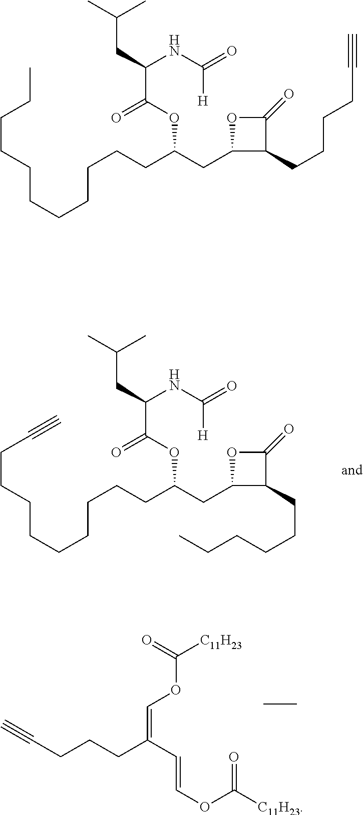

It is understood that the herein provided hydrolase inhibitors normally relate to the hydrolase inhibitors as such, but that for immobilization hydrolase inhibitors need to be modified/adapted, e.g. in that the inhibitors contain an alkyne group. The thus modified/adapted hydrolase inhibitors are also termed “derivatives of hydrolase inhibitor” herein.

The modified/adapted hydrolase inhibitor (hydrolase inhibitor derivative) (in the form of the inhibitor before being attached to the solid phase) preferably is a hydrolase inhibitor containing an alkyne group and is selected from compounds having a structure selected from:

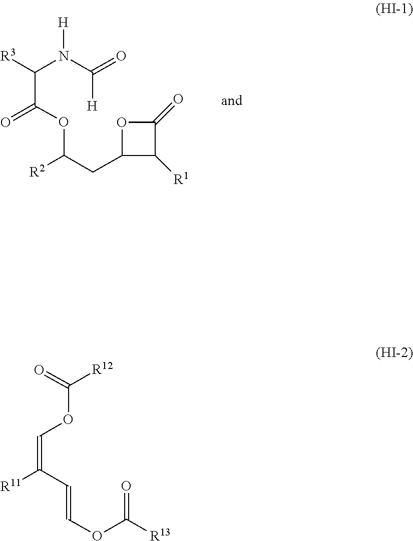

wherein in (HI-1)
R¹, R² and R³ are each independently selected from C₂₋₂₄-alkyl which may be linear or branched, wherein at least one terminal —CH₂—CH₃ group in R¹, R² or R³ is replaced by a —C≡CH group,

wherein in (HI-2)
R¹¹, R¹² and R¹³ are each independently selected from C₂₋₂₄-alkyl which may be linear or branched, and wherein at least one terminal —CH₂—CH₃ group in R¹¹, R¹² and R³ is replaced by a —C≡CH group.

Such modified/adapted hydrolase inhibitor (hydrolase inhibitor derivative) are also explained and defined herein further below.

In the following Markush structures are provided that show hydrolase inhibitors which may be used in context of the invention.

Markush: Orlistat:

Active structure element name: (Ethyloxethan-2-one):

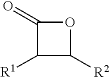

Orlistat as commercially available substance:

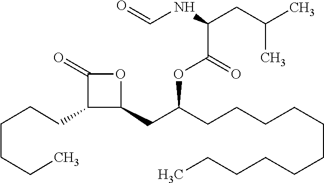 [SMILES: CCCCCCCCCCC[C@@H](C[C@@H]1OC(=O)[C@H]1CCCCCC)OC(=O)[C@H](CC(C)C)NC=O]

This is also referred to herein as formula (4).

R1=alkyl-alkin; R2=alkyl (Orlistat derivative 1; Orlistat A)):

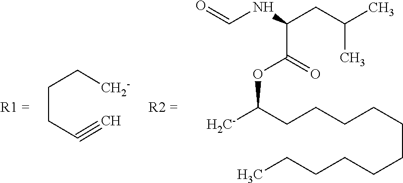

R1=alkyl; R2=alkyl-alkin (Orlistat derivative 2; Orlistat B)):

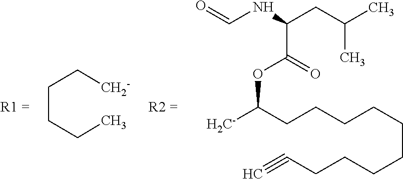

In a preferred embodiment the hydrolase inhibitor is orlistat. Preferably, orlistat is immobilized on a solid carrier via the reaction of an azido group and an alkyne group. It is understood that when Orlistat should be immobilized on a solid carrier an Orlistat derivative is to be used. Preferably, the Orlistat derivative comprises the alkyne group. Orlistat is also disclosed in Yang et al., (2010), J. Am. Chem. Soci 132, 656-666. Preferably, the Orlistat derivative comprising an alkyne group is selected from the group consisting of formulas (1), which is named Orlistat A herein and (2), which is named Orlistat B herein. The terms compound (A), formula (1), Orlistat A and Orlistat derivative 1 are used interchangeably herein. The terms compound (B), formula (2), Orlistat B and Orlistat derivative 2 are used interchangeably herein.

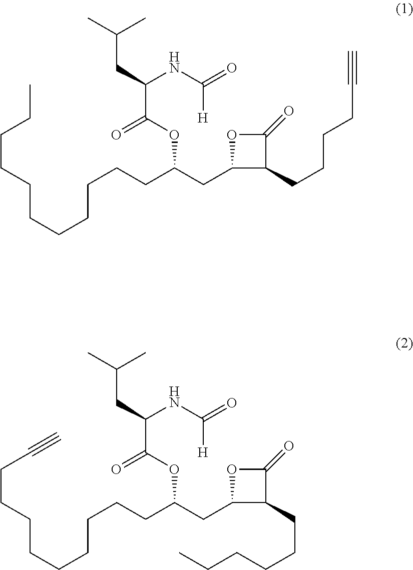 [SMILES: C#CCCCC[C@@H]1C(=O)O[C@H]1C[C@H](CCCCCCCCCCC)OC(=O)[C@@H](CC(C)C)NC=O ; C#CCCCCCCCCC[C@@H](C[C@@H]1OC(=O)[C@H]1CCCCCC)OC(=O)[C@@H](CC(C)C)NC=O]

In another preferred embodiment the hydrolase inhibitor is a bis-enol-ester. Bis-enol-esters are well known and described inter alia in “Functionalized Bis-enol Acetates as Specific Molecular Probes for Esterases” (Richter (2013) ChemBioChem 18:2435-2438).

This structural element (bis-enol-ester) originated from a natural compound having the structure of

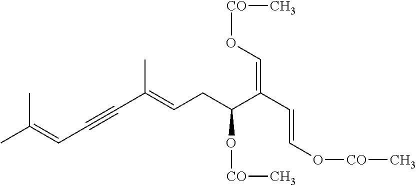 [SMILES: CC(=O)O/C=C(/C=C/OC(C)=O)[C@H](C/C=C(\C)C#CC=C(C)C)OC(C)=O]

(see https://roempp.thieme.de/lexicon/RD-03-00698 or https://pubchem.ncbi.nlm.nih.gov/compound/Caulerpenyne). This compound is known as Caulerpenyne with the CAS-Number 70000-22-5 and the PubChem CID thereof is 5311436.

The following relates to bis-enol-ester.

Markush: Bis-enol-ester:

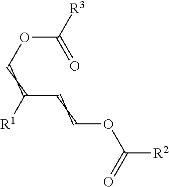

Active structure element name: (1,3)-buta-1,3-diene-1,4-diyl di-fatty acid
R1=H or coupling group [CH₂—(CH₂)ₙ-Alkin or DiG/<Dig>/Biotin-Streptavidin, NHS, SH,
LNA [n=1-10]
R2=—(CH2)x-CH3 [x=1-20]
R3=—(CH2)y-CH3 [y=1-20]

Preferably, the bis-enol-ester is immobilized on a solid carrier via a linker, such as alkyne group, e.g. via the reaction of an azido group and an alkyne group. It is understood that when the bis-enol-ester should be immobilized on a solid carrier a derivative of the bis-enol-ester is to be used. Preferably, the bis-enol-ester derivative comprises the alkyne group. Preferably, the bis-enol-ester derivative comprising an alkyne group comprises or consists of formula (3)

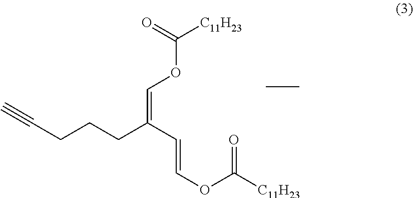

The terms compound (C) and formula (3) are used interchangeably herein.

As the skilled person will understand, expressions such as “immobilized inhibitor is selected from the group consisting of orlistat or bis-enol-ester” indicate that the orlistat or bis-enol-ester is linked to the solid carrier via suitable coupling group. For example, the orlistat or bis-enol-ester may be immobilized by reacting a modified orlistat or bis-enol-ester containing an alkyne group with a solid carrier having one or more azide groups. In this case, it is to be understood that the orlistat or bis-enol-ester as present on the solid carrier has a modification in one of its alkyl groups by means of which the orlistat or bis-enol-ester has been linked to the solid carrier. For example, if a CH2-CH3 group of the orlistat has been modified to a —C≡CH group to enable the linkage to the solid carrier by means of a “click” reaction with an azide group of the solid carrier, this part of the orlistat will also be modified in the form in which the orlistat is linked to the solid carrier (in particular the part which represented a —C≡CH group would become part of the triazole formed by the reaction of the —C≡CH group with the azide group).

As already mentioned above, the invention further relates in one aspect to a method for preparing or for obtaining a hydrolase inhibitor immobilized on a solid carrier and/or for preparing or for obtaining a device (such as a column) comprising a hydrolase inhibitor immobilized on a solid carrier. Accordingly, such a method comprises a step of immobilizing the hydrolase inhibitor on a solid carrier. The invention also relates in one aspect to a hydrolase inhibitor immobilized on a solid carrier and/or a device (such as a column) comprising a hydrolase inhibitor immobilized on a solid carrier. Said hydrolase inhibitor immobilized on a solid carrier and/or said device can be prepared or obtained by that method. Moreover, the immobilized inhibitor prepared, obtained or obtainable by the methods disclosed herein and/or a hydrolase inhibitor immobilized on a solid carrier prepared, obtained or obtainable by the methods disclosed herein can be used in accordance with the invention, e.g. in a method comprising the steps of
(i) contacting a starting composition comprising a protein with a hydrolase inhibitor, wherein the hydrolase inhibitor is immobilized on a solid carrier;
(ii) recovering a composition comprising the protein.

In a preferred aspect, the inhibitor is a group obtainable by reacting a compound comprising or consisting of formula (1), (2), (3) or (4), preferably formula (1), (2) or (4) with an azide.

In other words, the present invention also relates to the methods and the products expressed in the following items 1 to 42:

Item 1. A method for immobilizing a hydrolase inhibitor on a solid carrier, comprising the steps:
providing a solid carrier,
providing a hydrolase inhibitor,
contacting the solid carrier with a solution containing the hydrolase inhibitor, and
allowing the hydrolase inhibitor to be immobilized on the solid carrier.

Item 2. The method according to item 1, wherein the solid carrier is selected from poly(meth)acrylates like polymethylmethacrylate (PMMA), polystyrene, polyethylene oxide, cellulose and cellulose derivatives, e.g., cellulose acetate (CA) or regenerated cellulose, agarose, including crosslinked agarose, polysulfone (PSU), polyethersulfone (PES), polyethylene (PE), polypropylene (PP), polycarbonate (PC), polyacrylonitrile (PAN), polyamide (PA), polytetrafluoroethylene (PTFE), and blends or copolymers of the foregoing, or blends or copolymers with hydrophilizing polymers, preferably with polyvinylpyrrolidone (PVP) or polyethyleneoxide (PEO).

Item 3. The method according to item 1 or 2, wherein the step of providing the solid carrier includes a step of treating the solid carrier to introduce functional groups preferably selected from hydroxyl groups, carboxylate groups, ketones, aldehydes, isocyanates, epoxides, hydroxyl groups, carboxylate groups, amines, alkynes, azides, and biopolymers (e.g. streptavidin and/or biotin).

Item 4. The method according to item 3, wherein the step of providing the hydrolase inhibitor includes a step of treating the hydrolase inhibitor to introduce functional groups capable of reacting with the functional groups of the solid carrier to preferably form a streptavidin-biotin interaction or to form one or more groups selected from esters, amides, imines, urethanes, ureas, β-amino alcohols and 1,2,3-triazoles.

Item 5. The method according to any one of items 1 to 4, wherein the step of providing the hydrolase inhibitor includes a step of treating the hydrolase inhibitor to introduce functional groups preferably selected from hydroxyl groups, carboxylate groups, ketones, aldehydes, isocyanates, epoxides, hydroxyl groups, carboxylate groups, amines, alkynes, azides, and biopolymers (e.g. streptavidin and/or biotin).

Item 6. The method according to item 5, wherein the step of providing the solid carrier includes a step of treating the solid carrier to introduce functional groups capable of reacting with the functional groups of the hydrolase inhibitor to preferably form a streptavidin-biotin interaction or to form one or more groups selected from esters, amides, imines, urethanes, ureas, β-amino alcohols and 1,2,3-triazoles.

Item 7. The method according to any of the preceding items, wherein the method comprises the steps:
providing a solid carrier,
contacting the solid carrier with a solution containing a linker to form a carrier-linker,
providing a hydrolase inhibitor,
contacting the carrier-linker with a solution containing the hydrolase inhibitor, and
allowing the hydrolase inhibitor to be immobilized on the carrier-linker.

Item 8. The method according to item 7, wherein the step of providing the solid carrier includes a step of treating the solid carrier to introduce functional groups preferably selected from hydroxyl groups, carboxylate groups, ketones, aldehydes, isocyanates, epoxides, hydroxyl groups, carboxylate groups, amines, alkynes, azides and biopolymers (e.g. streptavidin and/or biotin).

Item 9. The method according to item 7 or 8, wherein the linker comprises functional groups capable of reacting with the functional groups of the solid carrier to preferably form a streptavidin-biotin interaction or to form one or more groups selected from esters, amides, imines, urethanes, ureas, β-amino alcohols and 1,2,3-triazoles.

Item 10. The method according to any one of items 7 to 9, wherein the step of providing the hydrolase inhibitor includes a step of treating the hydrolase inhibitor to introduce functional groups preferably selected from hydroxyl groups, carboxylate groups, ketones, aldehydes, isocyanates, epoxides, hydroxyl groups, carboxylate groups, amines, alkynes, azides and biopolymer (e.g streptavidin and/or biotin).

Item 11. The method according to any one of items 7 to 10, wherein the linker further comprises functional groups capable of reacting with the functional groups of the hydrolase inhibitor to preferably form a streptavidin-biotin interaction or to form one or more groups selected from esters, amides, imines, urethanes, ureas, β-amino alcohols and 1,2,3-triazoles.

Item 12. The method according to any of the preceding items, wherein the method comprises the steps:
providing a solid carrier,
providing a hydrolase inhibitor,
contacting the hydrolase inhibitor with a linker to form a hydrolase inhibitor-linker,
contacting the solid carrier with a solution containing the hydrolase inhibitor-linker, and
allowing the hydrolase inhibitor-linker to be immobilized on the solid carrier.

Item 13. The method according to item 12, wherein the step of providing the solid carrier includes a step of treating the solid carrier to introduce functional groups preferably selected from hydroxyl groups, carboxylate groups, ketones, aldehydes, isocyanates, epoxides, hydroxyl groups, carboxylate groups, amines, alkynes, azides and biopolymers (e.g. streptavidin and/or biotin).

Item 14. The method according to item 12 or 13, wherein the linker comprises functional groups capable of reacting with the functional groups of the solid carrier to preferably form a streptavidin-biotin interaction or to form one or more groups selected from esters, amides, imines, urethanes, ureas, β-amino alcohols and 1,2,3-triazoles.

Item 15. The method according to any one of items 12 to 14, wherein the step of providing the hydrolase inhibitor includes a step of treating the hydrolase inhibitor to introduce functional groups preferably selected from hydroxyl groups, carboxylate groups, ketones, aldehydes, isocyanates, epoxides, hydroxyl groups, carboxylate groups, amines, alkynes, azides and biopolymers (e.g. streptavidin and/or biotin).

Item 16. The method according to any one of items 12 to 15, wherein the linker further comprises functional groups capable of reacting with the functional groups of the hydrolase inhibitor to preferably form a streptavidin-biotin interaction or to form one or more groups selected from esters, amides, imines, urethanes, ureas, β-amino alcohols and 1,2,3-triazoles.

Item 17. The method according to any one of items 1 to 16, wherein the functional groups on the solid carrier comprise amino groups, preferably primary amino groups.

Item 18. The method according to item 17, wherein the amino groups on the solid carrier are introduced by treatment with a reactive plasma, preferably a plasma generated from a gas mixture comprising ammonia.

Item 19. The method according to any of items 7 to 18, wherein the linker is a compound containing at least two functional groups selected from hydroxyl groups, carboxylate groups, ketones, aldehydes, isocyanates, epoxides, hydroxyl groups, carboxylate groups, amines, alkynes, azides and biopolymers (e.g. streptavidin and/or biotin).

Item 20. The method according to item 19, wherein the linker furthermore contains a polyoxyethylene or polyoxypropylene moiety, preferably containing 3 to 20 (more preferably 3 to 10, even more preferably 3 to 5) oxyethylene or oxypropylene units, to which the at least two functional groups are bound.

Item 21. The method according to any of items 7 to 20, wherein the linker contains at least two different functional groups selected from hydroxyl groups, carboxylate groups, ketones, aldehydes, isocyanates, epoxides, hydroxyl groups, carboxylate groups, amines, alkynes, azides and biopolymers (e.g. streptavidin and/or biotin).

Item 22. The method according to item 21, wherein the linker contains per molecule a first functional group selected from hydroxyl groups, carboxylate groups, ketones, aldehydes, isocyanates, epoxides, hydroxyl groups, carboxylate groups, amines, alkynes, azides, streptavidin and biotin and furthermore at least two second functional groups, the at least two second functional groups preferably being of the same type, selected from hydroxyl groups, carboxylate groups, ketones, aldehydes, isocyanates, epoxides, hydroxyl groups, carboxylate groups, amines, alkynes, azides and biopolymers (e.g. streptavidin and/or biotin) which are different from the first functional group.

Item 23. The method according to any of items 1 to 22, wherein the solid carrier is crosslinked agarose, preferably sepharose, more preferably Mag-Sepharose-Streptavidin or Streptavidin Sepharose® High Performance beads.

Item 24. The method according to any of items 1 to 23, wherein the solid carrier contains a functional group including streptavidin and the linker contains a functional group including biotin.

Item 25. The method according to item 24, wherein the linker furthermore contains an azido group and the hydrolase inhibitor contains an alkyne group.

Item 26. The method according to any of items 1 to 25, wherein the solid carrier contains a functional group including a carboxylate, which is optionally NHS-activated, and the linker contains a functional group including an amine.

Item 27. The method according to item 26, wherein the linker furthermore contains an azido group and the hydrolase inhibitor contains an alkyne group.

Item 28. The method according to any of items 1 to 27, wherein the linker is selected from Biotin-PEG3-Azide (CAS 875770-34-6), Biotin-PEG4-Azide (CAS 1309649-57-7), Azo-Biotin-Azide (CAS 1339202-33-3) and Azido-PEG4-Amine (CAS 951671-92-4).

Item 29. The method according to any one of items 1 to 28, wherein the hydrolase inhibitor contains an alkyne group and is selected from compounds having a structure selected from:

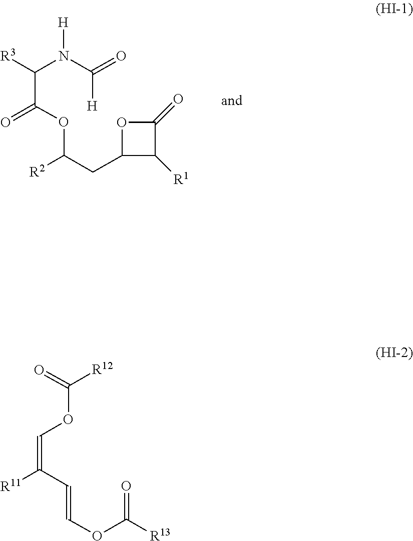

wherein in (HI-1)
R¹, R² and R³ are each independently selected from C₂₋₂₄-alkyl which may be linear or branched, wherein at least one terminal —CH₂—CH₃ group in R¹, R² or R³ is replaced by a —C≡CH group,

wherein in (HI-2)
R¹¹, R¹² and R¹³ are each independently selected from C₂₋₂₄-alkyl which may be linear or branched, and wherein at least one terminal —CH₂—CH₃ group in R¹¹, R¹² and R¹³ is replaced by a —C≡CH group.

Item 30. The method according to item 29, wherein the number of carbon atoms in R¹, R² and R¹¹ is independently selected from 2 to 15, preferably 3 to 14, more preferably 4 to 12, even more preferably from 5 to 11.

Item 31. The method according to item 29 or 30, wherein the number of carbon atoms in R¹² and R¹³ is independently selected from 5 to 24, preferably 8 to 18, more preferably 10 to 16, even more preferably from 10 to 12.

Item 32. The method according to any one of items 29 to 31, wherein the number of carbon atoms in R³ is selected from 2 to 10, preferably 2 to 8, more preferably 3 to 6, even more preferably from 3 to 5.

Item 33. The method according to any one of items 29 to 32, wherein the alkyl groups of R¹ and R² are linear.

Item 34. The method according to any one of items 29 to 33, wherein the alkyl groups of R¹¹ R¹² and R¹³ are linear.

Item 35. The method according to any one of items 29 to 34, wherein the alkyl group of R³ is branched.

Item 36. The method according to any one of items 29 to 35, wherein formula (HI-1) has the following structure (HI-1a):

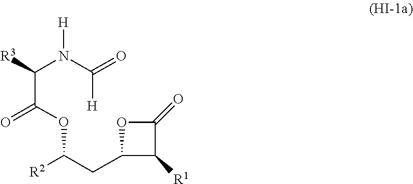

wherein R¹, R² and R³ are as defined in the preceding items.

Item 37. The method according to any one of items 1 to 36, wherein the hydrolase inhibitor contains an alkyne group and is selected from compounds having a structure selected from:

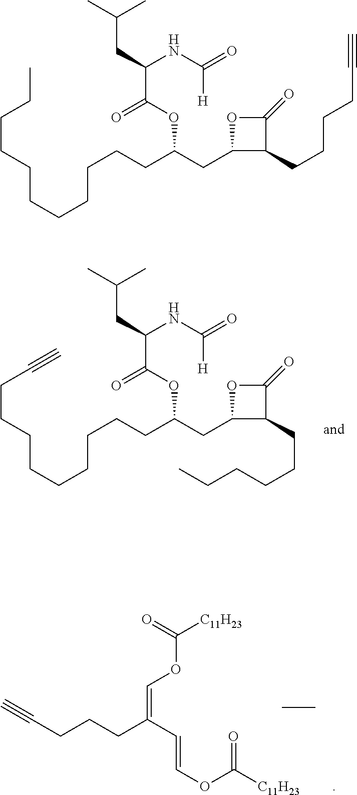

Item 38. A hydrolase inhibitor immobilized on a solid carrier which is obtainable by the method according to any one of items 1 to 37.

Item 39. A device comprising the hydrolase inhibitor immobilized on a solid carrier according to item 38.

Item 40. The device according to item 39, wherein the device is a tubular device having at least two openings.

Item 41. The device according to item 39 or 40, wherein the device is comprised of a container, preferably made of a metal, polymer or glass, the container forming a cavity in which the hydrolase inhibitor immobilized on a solid carrier is contained.

Item 42. The device according any one of items 39 to 41, wherein the device is a column, which is at least partially filled with the hydrolase inhibitor immobilized on a solid carrier.

It is preferred that a starting composition comprising a protein is contacted with a hydrolase inhibitor immobilized on a solid carrier before the composition comprising the protein is recovered. In other words, the composition comprising the protein is recovered after the starting composition comprising the protein is contacted with a hydrolase inhibitor immobilized on a solid carrier. Accordingly, the method as described herein may be carried out in the following order:
step (i) contacting a starting composition comprising a protein with a hydrolase inhibitor, wherein the hydrolase inhibitor is immobilized on a solid carrier;

followed by
step (ii) recovering a composition comprising the protein.

The steps (i) and (ii) of the methods described herein are typically to be performed in that order, i.e. step (i) is followed by step (ii). While there might be further steps (such as further purification steps) between step (i) and step (ii), it is preferred herein that step (i) is directly followed by step (ii), i.e. that there is no further step between step (i) and step (ii).

Moreover, it is envisaged and preferred herein that steps (i) and (ii) (preferably in the order (i) followed by (ii)) can be performed once, e.g. a starting composition comprising a protein, like a crude extract or an extract that has been purified by conventional methods (such as Protein A affinity chromatography), is subject to step (i) of the method followed by recovery of the composition comprising the protein according to step (ii). In other words, it is preferred herein that step (i) is performed (solely) once in the herein-described method/when carrying out the method. As mentioned and described herein, the method may comprise additional steps of purification and/or preparation prior to step (i) and/or between steps (i) and (ii), e.g. affinity chromatography, ion chromatography and/or mixed mode chromatography.

However, it is also envisaged that steps (i) and (ii) (preferably in the order (i) followed by (ii)) can be repeated one or more times, for example 2, 3, 4, 5, 6, 7, 8, 9 or 10 times. For example, it might be desired to (further) reduce the hydrolytic activity in the recovered composition comprising the protein after steps (i) and (ii) have been performed a first time, for example, to achieve a certain threshold of hydrolytic activity and/or in order to (further) remove or reduce impurities, such as host cell protein(s), particularly hydrolases.

In this context, the composition comprising the protein recovered in step (ii) of the method would be contacted with a hydrolase inhibitor, wherein the hydrolase inhibitor is immobilized on a solid carrier.

For example, the method could then accordingly be carried out as follows (preferably in the order (i) followed by (ii) followed by (iii) followed by (iv) (short order (i) to (iv):

A method comprising the steps of
(i) contacting a starting composition comprising a protein with a hydrolase inhibitor, wherein the hydrolase inhibitor is immobilized on a solid carrier;
(ii) recovering a composition comprising the protein;
(iii) contacting the composition comprising the protein recovered in the (preceding) step (ii) with a hydrolase inhibitor, wherein the hydrolase inhibitor is immobilized on a solid carrier;
(iv) recovering a composition comprising the protein.

This could then be repeated as desired, as described above, for example as follows:

A method comprising the steps of
(i) contacting a starting composition comprising a protein with a hydrolase inhibitor, wherein the hydrolase inhibitor is immobilized on a solid carrier;
(ii) recovering a composition comprising the protein;
(iii) contacting the composition comprising the protein recovered in the (preceding) step (ii) with a hydrolase inhibitor, wherein the hydrolase inhibitor is immobilized on a solid carrier;
(iv) recovering a composition comprising the protein;
(v) contacting the composition comprising the protein recovered in the (preceding) step (iv) with a hydrolase inhibitor, wherein the hydrolase inhibitor is immobilized on a solid carrier;
(vi) recovering a composition comprising the protein;
etc.

For further repetitions of the steps, any desired number of the following steps could be additionally performed in the method:
(vii) contacting the composition comprising the protein recovered in the preceding step with a hydrolase inhibitor, wherein the hydrolase inhibitor is immobilized on a solid carrier;
(viii) recovering a composition comprising the protein;
etc.

Although clear for the skilled person it is pointed out that the method described herein may be combined with means and methods of (conventional) protein preparation and/or purification.

In other words, the step of contacting a starting composition comprising a protein with an immobilized hydrolase inhibitor and the step of recovering a composition the protein can be combined with additional steps of (conventional) protein preparation and/or purification.

Accordingly, in a preferred aspect, the method may further comprise one or more steps of (conventional) protein preparation and/or purification prior to step “(i) contacting a starting composition comprising a protein with a hydrolase inhibitor, wherein the hydrolase inhibitor is immobilized on a solid carrier”.

The method may further comprise one or more steps of (conventional) protein preparation and/or purification after step “(i) contacting a starting composition comprising a protein with a hydrolase inhibitor, wherein the hydrolase inhibitor is immobilized on a solid carrier”.

The method may further comprise one or more steps of (conventional) protein preparation and/or purification prior to and after step (i) contacting a starting composition comprising a protein with a hydrolase inhibitor, wherein the hydrolase inhibitor is immobilized on a solid carrier.

Non-limiting examples for (conventional) protein preparation and/or purification steps are ion exchange chromatography (such as anion exchange chromatography (AEX) and/or cation exchange chromatography (CEX), preferably cation exchange chromatography (CEX)), affinity chromatography, hydrophobic interaction chromatography (HIC) and (ultra)filtration, or combinations thereof such as mixed mode chromatography (e.g. mixed mode ion exchange chromatography, preferably mixed mode anion exchange chromatography (MMAEX)). Affinity chromatography preferably is Protein A affinity chromatography.

The meaning of these terms and protein preparation and/or purification steps, including affinity chromatography, ion exchange chromatography (such as anion exchange chromatography (AEX) and/or cation exchange chromatography (CEX), and/or hydrophobic interaction chromatography (HIC) chromatography, are well known in the art.

The advantage of mixed mode chromatography is that the solutes interact with the stationary phase through more than one interaction mode or mechanism resulting in higher selectivity and separation power (Zhang and Liu (2016) J. Pharm. Biomed. Anal. 128:73-88). Mixed mode chromatography (or multimodal chromatography) can for example comprise the following methods:

IEC/HIC

Since IEC and HIC conditions are the closest ones to physiological conditions which are fit for maintaining biological activity, the combinations of them are widely used in the separation of biological products. IEC/HIC MMC has improved separation power and selectivity on the grounds that the solutes interact with the stationary phase through both electrostatic and hydrophobic interactions.

IEC/RPLC

IEC/RP MMC combines the advantages of RPLC and IEC. For example, WAX/RP has increased separation power and degree of freedom in adjusting the separation selectivity when compared with single WAX or RPLC-

HILIC/RPLC

Liu et al. synthesized a HILIC/RP stationary phase which could show RPLC or HILIC retention by adjusting the organic phase in mobile phase (Liu and Pohl (2008) J. Chrom. A 1191:83-89.)

HILIC/IEC

Mant et al. reported that HILIC/CEX offered unique selectivity, stronger separation power and wider range of applications compared to RPLC for peptide separations. (Mant et al. (1998) J. Chrom. A. 816 (1):79-88)

SEC/IEC

Hydrophobic interactions in protein SEC are relatively weak at low ionic strength, electrostatic effects may contribute significantly to retention, and this allows us to use an SEC column as a weak ion exchanger.

Preferably, contacting a starting composition comprising a protein with a hydrolase inhibitor is carried out after the starting composition comprising a protein was (solely) subjected to Protein A affinity chromatography, and, optionally, filtration. If filtration is carried out, preferably the order is Protein A affinity chromatography and subsequently filtration (i.e. Protein A affinity chromatography followed by filtration). “(Solely) subjected” means that the starting composition was only subjected to a given (conventional) protein preparation and/or purification steps, such as, in this context Protein A affinity chromatography, and, optionally, filtration, i.e. the method does not comprise any other purification method than the given one(s) prior to step (i).

It is envisaged herein that contacting a starting composition comprising a protein with a hydrolase inhibitor is carried out after the starting composition comprising a protein was (solely) subjected to Protein A affinity chromatography, and, optionally, filtration, and/or after the starting composition comprising a protein was subjected to ion exchange chromatography or mixed mode ion exchange chromatography, preferably cation exchange chromatography or mixed mode anion exchange chromatography.

Accordingly, in a preferred aspect, the method may further comprise affinity chromatography (preferably Protein A affinity chromatography) and, optionally, filtration, prior to step (i) contacting a starting composition comprising a protein with a hydrolase inhibitor, wherein the hydrolase inhibitor is immobilized on a solid carrier.

Protein A affinity chromatography as used herein preferably includes a decrease of the pH value of the composition and a subsequent increase of the pH value (to the initial pH value). This serves to inactivate viruses (if (potentially) present in the composition). A composition that was subject to such a protein A affinity chromatography is also termed “conditioned protein A solution pool”, “conditioned protein A elution pool”, “conditioned Protein A eluate”, protein A elution pool or protein A eluate herein.

Further, filtration as used herein can include using a depth filter. Accordingly, depth filtration is preferred herein. A composition that was subject to such a protein A affinity chromatography followed by filtration is also termed “mixed mode anion exchange load solution” herein (which indicates that the solution is—after carrying out step (i)—subject to mixed mode ion exchange chromatography, particularly mixed mode anion exchange chromatography.

As mentioned above, the method may further comprise one or more steps of (conventional) protein preparation and/or purification after step “(i) contacting a starting composition comprising a protein with a hydrolase inhibitor, wherein the hydrolase inhibitor is immobilized on a solid carrier”. In a preferred aspect, the method may further comprise (at least) one step of ion exchange chromatography and/or mixed mode chromatography (such as mixed mode ion exchange chromatography) and/or hydrophobic interaction chromatography (HIC), preferably of cation exchange chromatography and/or mixed mode chromatography (such as mixed mode anion exchange chromatography) and/or hydrophobic interaction chromatography (HIC), after step (i) contacting a starting composition comprising a protein with a hydrolase inhibitor, wherein the hydrolase inhibitor is immobilized on a solid carrier (and prior to the recovery of the composition).

Mixed mode ion exchange chromatography preferably herein is mixed mode anion exchange chromatography (MMAEX).

In some embodiments, the steps used for producing, purifying, and/or separating the protein of interest from the culture cells, as disclosed herein, further comprise at least one of steps selected from the group consisting of: a harvest clarification process (or a similar process to remove the intact cells and cell debris from the cell culture), an ultrafiltration (UF) process (or a similar process to concentrate the produced protein), a diafiltration (DF) process (or a similar process to change or dilute the buffer comprising the produced protein from previous processes), a viral inactivation process (or a similar process to inactivate or remove viral particles), an affinity capture process (or any one of chromatography methods to capture the produced protein and separate it from the rest of the buffer/solution components), a formulation process and a bulk fill process. In one aspect, the steps for producing, purifying, and/or separating the protein from the culture cells, as disclosed herein, comprise at least a harvest clarification process (or a similar process to remove the intact cells and cell debris from the cell culture), a post-harvest ultrafiltration (UF) process (or a similar process to concentrate the produced protein), a post-harvest diafiltration (DF) process (or a similar process to change or dilute the buffer comprising the produced protein from previous processes), a solvent/detergent viral inactivation process (or a similar process to chemically inactivate viral particles), an intermediate purification process (such as hydrophobic interaction chromatography (HIC) or any one of chromatography methods to capture the produced protein and separate it from the rest of the buffer/solution components), a post-HIC UF/DF process (or a similar process to concentrate and/or buffer exchange for the produced protein), a viral reduction filtration process (or a similar process to further remove any viral particles or other impurities or contaminants); a mixed-mode chromatography (such as CAPTO® Adhere agarose chromatography, or a similar process to further purify and/or concentrate the produced protein), a formulation process and a bulk fill process. In one aspect, the separating step of the method provided herein further comprises at least one of harvest clarification, ultrafiltration, diafiltration, viral inactivation, affinity capture, HIC chromatography, mixed-mode chromatography and combinations thereof.

In some aspects, the steps used for purifying the protein from the culture cells, as disclosed herein, comprise a chromatography process. In one embodiment, the chromatography process is an affinity capture process. The chromatography process may involve, at least, a Hydrophobic Interaction Chromatography (HIC), a Protein A chromatography, or a CAPTO® Adhere mixed-mode chromatography.

As mentioned above in a preferred aspect, the method described herein may further comprise one or more steps of (conventional) protein preparation and/or purification prior to and/or after step “(i) contacting a starting composition comprising a protein with a hydrolase inhibitor, wherein the hydrolase inhibitor is immobilized on a solid carrier”. Accordingly, the method described herein can be a platform process (i.e. a method/process for purifying a protein of interest, such as an antibody).

Such a method/process may comprise the use of several columns with different purification principles (i.e. different protein preparation and/or purification steps).

A typical process or method described herein (for preparing a composition comprising a protein) comprises at least 4 subsequent steps of (conventional) preparation and/or purification of a protein of interest, i.e. preparing a protein composition from the cell culture, followed by typically (at least) 3 (conventional) chromatographic steps, optionally 4 (conventional) chromatographic steps.

Thus: after a protein composition has been prepared from the cell culture or culture supernatant (step 1), a method/process typically involves 3 subsequent (conventional) chromatographic steps (optionally 4 subsequent steps), e.g. normally using 3 columns (optionally 4 columns) each employing a different purification principle (steps 2-4, or if an optional step 5 (column 4) is included, steps 2-5).

Preferably, the method/process described herein (for preparing a composition comprising a protein) comprises the use of (at least) three columns (optionally 4 columns) with three (optionally 4) different purification principles in addition to step “(i) contacting a starting composition comprising a protein with a hydrolase inhibitor, wherein the hydrolase inhibitor is immobilized on a solid carrier”.

FIG. 7 shows a conventional schematic method/process for the preparation of a composition of a protein of interest/purification of a protein of interest. FIG. 7 also illustrates where the step “(i) contacting a starting composition comprising a protein with a hydrolase inhibitor, wherein the hydrolase inhibitor is immobilized on a solid carrier” can be employed in a method of the invention.

First, a composition from the cell culture (cells producing the protein/protein of interest) is prepared, e.g. by homogenizing the cells. The composition may also be a cell culture supernatant. Preferably, the above composition is subsequently purified, e.g. the composition then is a composition (as) separated (e.g. by filtration) from cells, cell debris and/or aggregates. Accordingly, the (subsequently purified) composition is a composition (as) separated (by filtration) from cells, cell debris and/or aggregates, e.g. is Harvest Cell Culture Fluid (HCCF).

Second, the above composition is further purified, e.g. by a further step of protein preparation and/or purification (step 2). The further step of protein preparation and/or purification preferably is affinity chromatography (preferably Protein A affinity chromatography). Hence, affinity chromatography (preferably Protein A affinity chromatography) is preferably the first chromatographic step. For example, the above composition (e.g. the Harvest Cell Culture Fluid (HCCF)) is subjected to Protein A affinity chromatography. In other words, the process includes the use of an affinity chromatography column (Protein A affinity chromatography column).

Third, the eluate is then subjected to a second column (Column 2)/step 3).

Fourth, the eluate of the second column is then subjected to a third column (Column 3/step 4).

Fifth, if the quality (for example purity of the protein of interest) is not achieved or is to be further improved after the fourth step (i.e. after three columns (preferably each employing a different purification principle)), optionally a fifth step (e.g. a fourth column with a fourth purification principle), can be used (Column 4/step 5). The skilled person can decide on a case by case basis whether a fifth step is necessary and/or beneficial e.g. when balancing costs and/or increased complexity versus improved quality.

Thus, steps 3 to 5 can be ion exchange chromatography (e.g. anion exchange chromatography (AEX) and/or cation exchange chromatography (CEX)), mixed mode chromatography and hydrophobic interaction chromatography (HIC) in any order. In other words, columns 2 to 4 can be ion exchange chromatography column (e.g. anion exchange chromatography (AEX) column or cation exchange chromatography (CEX) column), mixed mode chromatography column and hydrophobic interaction chromatography (HIC) column in any order, i.e., the process includes the use of any of the above columns in any order.

During development of a platform process/purification process the skilled person can determine the order of the steps/of the columns to be used.

Sixth, after Column 3 or the optional Column 4 (step 4 and, optionally step 5) the eluate is subjected to virus filtration and then, seventh, subjected to ultrafiltration/diafiltration (UFDF).

After the seventh step (UFDF) the composition comprising the protein can be recovered.

In the scheme of FIG. 7 the Harvest Cell Culture Fluid (HCCF) is subjected to Protein A affinity chromatography. The eluate is then subjected to a second column (Column 2). The eluate of the second column is then subjected to a third column (Column 3). If the quality (for example purity of the protein of interest) is not achieved after a third step (i.e. after three columns with three different purification principles), a fifth step (e.g. a fourth column with a fourth purification principle), can be used (Column 4 in FIG. 7), e.g. if there is a real need. Otherwise, the fifth step/fourth column would normally not be used to avoid cost, material loss and complexity increase. The skilled person can decide on a case by case basis whether a fourth step is necessary and/or beneficial.

For column 2, 3 and 4 as shown in FIG. 7 there is a sequence defined during development. Purification principles that are evaluated are described herein and may be ion exchange chromatography (e.g. anion-cation-exchange chromatography), mixed mode chromatography or hydrophobic interaction chromatography. In other words, during development of a platform process/purification process the skilled person will determine the order of the columns used. Several non-limiting examples of the order of different purification principles (protein preparation and/or purification steps) relative to each other are provided herein above.

After Column 3 or the optional Column 4 the eluate is subjected to virus filtration and then subjected to ultrafiltration/diafiltration (UFDF).

Furthermore, the skilled person can determine when to perform the step “(i) contacting a starting composition comprising a protein with a hydrolase inhibitor, wherein the hydrolase inhibitor is immobilized on a solid carrier” (termed contacting composition with a hydrolase inhibitor in FIG. 7).

The method/process of the invention (for preparing a composition comprising a protein) comprises a step “(i) contacting a starting composition comprising a protein with a hydrolase inhibitor (such as Orlistat), wherein the hydrolase inhibitor is immobilized on a solid carrier” (e.g. the step is hydrolase inhibitor chromatography (a hydrolase inhibitor chromatographic step). In other words, the method/process can include the use of a hydrolase inhibitor (e.g. Orlistat) column.

Preferably, the hydrolase inhibitor chromatography/chromatographic step follows the step of affinity chromatography (preferably protein A affinity chromatography)/step 2. In other words, the process can include the use of a hydrolase inhibitor (e.g. Orlistat) column (directly) following the protein A column. Thus, preferably, step “(i) contacting a starting composition comprising a protein with a hydrolase inhibitor, wherein the hydrolase inhibitor is immobilized on a solid carrier” is performed after affinity chromatography (preferably Protein A affinity chromatography). This is to ensure that the following purification steps ensure removal of the inhibitor (such as orlistat) from the protein composition. The inhibitor might, for example, be present in the composition after performing said step (i) due to a potential bleeding of the matrix (i.e. bleeding from the solid carrier on which the inhibitor is immobilized).

However, as also discussed elsewhere herein step “(i) contacting a starting composition comprising a protein with a hydrolase inhibitor, wherein the hydrolase inhibitor is immobilized on a solid carrier” can be performed after Column 2 or Column 3 (if a fifth step is in place).

Thus, alternatively, the hydrolase inhibitor chromatography/chromatographic step can follow any one of steps 3 to 5 (steps 3 to 5 can be ion exchange chromatography (e.g. anion exchange chromatography (AEX) and/or cation exchange chromatography (CEX)), mixed mode chromatography and hydrophobic interaction chromatography (HIC) in any order). In other words, the process includes the use of a hydrolase inhibitor (e.g. Orlistat) column (directly) following the use of an ion exchange chromatography column (e.g. anion exchange chromatography (AEX) column or cation exchange chromatography (CEX) column), mixed mode chromatography column and hydrophobic interaction chromatography (HIC) column in any order.

In one aspect, the method/process does not comprise mixed mode chromatography.

The hydrolase inhibitor chromatography/chromatographic step normally does not (directly) follow the initial step of preparing the protein composition from the cell culture (e.g. does not follow HCCF). In other words, the hydrolase inhibitor chromatography/chromatographic step normally is not performed prior to affinity chromatography (e.g. protein A affinity chromatography)/step 1. This is because such an initial protein composition (e.g. protein composition from the cell culture/supernatant of the cell culture, preferably HCCF) typically still comprises a high degree of impurities, e.g. cells, cell debris and/or aggregates. Thus, the process normally does not include the use of a hydrolase inhibitor (e.g. Orlistat) column (directly) following the initial step of preparing the protein composition from the cell culture and/or does normally not include the use of a hydrolase inhibitor (e.g. Orlistat) column prior to the use of an affinity chromatographic column (e.g. protein A affinity chromatography column).

Independent of the order of the steps to be used in the method described herein, it is clear that the purified drug substance (the recovered composition comprising the protein) should not contain the inhibitor (e.g. Orlistat) or contain only minimal amounts (preferably non-detectable amounts) or amounts that do not prevent the administration of the composition (or a protein formulation described herein, e.g. a formulation comprising the composition) to subject/patients, e.g. in form of a formulation approved by the authorities such as FDA or EMA.

In other words, the method will be used in a way that ensures no inhibitor (or only minimal amounts as described above) is present in the recovered protein composition and/or a protein formulation described herein.

The method (for preparing a composition comprising a protein) described herein can comprise steps in the following order
(1) preparing a composition from the cell culture (cells producing the protein/protein of interest) or obtaining a cell culture supernatant from the cell culture, preferably followed by subsequent purification of the prepared composition or supernatant, e.g. separation/removal (e.g. by filtration) from cells, cell debris and/or aggregates (preferably the prepared and/or purified composition is Harvest Cell Culture Fluid (HCCF));
(2) affinity chromatography (preferably protein A affinity chromatography) (to be applied to the (purified) composition obtained by performing step (1)),
   followed by “(i) contacting a starting composition (e.g. here the (purified) composition obtained by performing affinity chromatography) comprising a protein with a hydrolase inhibitor (such as Orlistat), wherein the hydrolase inhibitor is immobilized on a solid carrier” (e.g. the step is hydrolase inhibitor chromatography (a hydrolase inhibitor chromatographic step);
(3) ion exchange chromatography (e.g. anion exchange chromatography (AEX) and/or cation exchange chromatography (CEX)) (to be applied to the (purified) composition obtained by performing step (2);
(4) mixed mode chromatography (to be applied to the (purified) composition obtained by performing step (3);

Optionally (5) hydrophobic interaction chromatography (HIC) (to be applied to the (purified) composition obtained by performing step (4);
(6) virus filtration (to be applied to the (purified) composition obtained by performing step (4) (or (5) if applicable);
(7) filtration (e.g. UFDF) (to be applied to the (purified) composition obtained by performing step (6); followed by (ii) recovering a composition comprising the protein.

As discussed, ion exchange chromatography (e.g. anion exchange chromatography (AEX) and/or cation exchange chromatography (CEX)), mixed mode chromatography and hydrophobic interaction chromatography (HIC) can be used in any order.

Thus, alternatively, the above described method can, for example, comprise steps in the following order:
(3) mixed mode chromatography (to be applied to the (purified) composition obtained by performing step (2);
(4) ion exchange chromatography (e.g. anion exchange chromatography (AEX) and/or cation exchange chromatography (CEX)) (to be applied to the (purified) composition obtained by performing step (3);

Optionally (5) hydrophobic interaction chromatography (HIC) (to be applied to the (purified) composition obtained by performing step (4).

In a further alternative, the above described method can, for example, comprise steps in the following order:
(3) hydrophobic interaction chromatography (HIC) (to be applied to the (purified) composition obtained by performing step (2);
(4) ion exchange chromatography (e.g. anion exchange chromatography (AEX) and/or cation exchange chromatography (CEX)) (to be applied to the (purified) composition obtained by performing step (3);

Optionally (5) mixed mode chromatography (to be applied to the (purified) composition obtained by performing step (4).

In a further alternative, the above described method can, for example, comprise steps in the following order:
(3) ion exchange chromatography (e.g. anion exchange chromatography (AEX) and/or cation exchange chromatography (CEX)) (to be applied to the (purified) composition obtained by performing step (2);
(4) hydrophobic interaction chromatography (HIC) (to be applied to the (purified) composition obtained by performing step (3);

Optionally (5) mixed mode chromatography (to be applied to the (purified) composition obtained by performing step (4).

As discussed elsewhere herein, step “(i) contacting a starting composition comprising a protein with a hydrolase inhibitor, wherein the hydrolase inhibitor is immobilized on a solid carrier” is preferably performed following affinity chromatography (see step (2) above).

As also discussed above, step “(i) contacting a starting composition comprising a protein with a hydrolase inhibitor, wherein the hydrolase inhibitor is immobilized on a solid carrier” can, in the alternative to following affinity chromatography (see step (2) above) (or even in addition to following affinity chromatography), be performed after/following ion exchange chromatography (e.g. anion exchange chromatography (AEX) and/or cation exchange chromatography (CEX)), or in a further alternative, after/following mixed mode chromatography, or in further alternative after/following hydrophobic interaction chromatography (HIC), in the methods described herein, e.g. in steps (3), (4) and/or (5) above.

In accordance with the term “starting composition” as used and defined herein, any of the above (purified) composition(s) which are to be contacted with a hydrolase inhibitor, wherein the hydrolase inhibitor is immobilized on a solid carrier (step (i)), is or can be a “starting composition”. For example, the (purified) composition obtained by performing affinity chromatography) (see step (2) above) is a “starting composition comprising a protein”.

The same explanation applies mutatis mutandis generally to the (purified) composition described herein to be contacted with a hydrolase inhibitor, wherein the hydrolase inhibitor is immobilized on a solid carrier (step (i)). For example, a (purified) composition obtained by performing ion exchange chromatography (e.g. anion exchange chromatography (AEX) and/or cation exchange chromatography (CEX)). mixed mode chromatography and/or hydrophobic interaction chromatography (HIC) is a “starting composition comprising a protein”.

Generally, it is understood that the term “contacting a starting composition comprising a protein with a hydrolase inhibitor” means that the “starting composition comprises a protein” and that this composition is to be contacted with a hydrolase inhibitor (wherein the hydrolase inhibitor is immobilized on a solid carrier). Accordingly, the starting composition comprises a protein and not a hydrolase inhibitor.

The following relates to a culture cell(s)/cell culture that can be used to prepare/produce the protein of interest. The nucleic acid encoding the protein of interest can be introduced into such a host cell as an exogenous gene, preferably with regulatory elements, already be present in the host cell as an active endogenous gene or become activated as an endogenous non-active gene. Corresponding culture cell(s) can be host cell(s) or a non-human host (cell) transformed with or comprising a vector or the nucleic acid molecule encoding the protein of interest. It will be appreciated that the term “host cell or a non-human host transformed with the vector of the invention”, in accordance with the present invention, typically relates to a host cell or a non-human host that comprises the vector or the nucleic acid molecule encoding the protein. Host cells for the expression of polypeptides are well known in the art and comprise prokaryotic cells as well as eukaryotic cells. Thus, the host can be selected from the group consisting of a bacterium, a mammalian cell, an algal cell, a ciliate, yeast and a plant cell. Typical bacteria include Escherichia, Corynebacterium (glutamicum), Pseudomonas (fluorescens), Lactobacillus, Streptomyces, Salmonella Bacillus (such as Bacillus megaterium or Bacillus subtilis), or Corynebacterium (like Corynebacterium glutamicum). The most preferred bacterium host herein is Escherichia coli (E. coli). An exemplary ciliate to be used herein is Tetrahymena, e.g. Tetrahymena thermophila.

Typical mammalian cells include, Hela, HEK293, HEK293T, H9, Per.C6 and Jurkat cells, mouse NIH3T3, NS0 and C127 cells, COS 1, COS 7 and CV1, quail QC1-3 cells, mouse L cells, mouse sarcoma cells, Bowes melanoma cells and Chinese hamster ovary (CHO) cells. Most preferred mammalian host cells in accordance with the present invention are CHO cells. Also, human embryonic kidney (HEK) cells are preferred.

Other suitable eukaryotic host cells are e.g. yeasts such as Pichia pastoris, Kluyveromyces lactis, Saccharomyces cerevisiae and Schizosaccharomyces pombe or chicken cells, such as e.g. DT40 cells. Insect cells suitable for expression are e.g. Drosophila S2, Drosophila Kc, Spodoptera Sf9 and SJ21 or Trichoplusia Hi5 cells. Preferable algal cells are Chlamydomonas reinhardtii or Synechococcus elongatus cells and the like. An exemplary plant is Physcomitrella, for example Physcomitrella patens.

Also within the scope of the present invention are primary mammalian cells or cell lines. Primary cells are cells which are directly obtained from an organism. Suitable primary cells are, for example, mouse embryonic fibroblasts (MEF), mouse primary hepatocytes, cardiomyocytes and neuronal cells as well as mouse muscle stem cells (satellite cells), human dermal and pulmonary fibroblasts, human epithelial cells (nasal, tracheal, renal, placental, intestinal, bronchial epithelial cells), human secretory cells (from salivary, sebaceous and sweat glands), human endocrine cells (thyroid cells), human adipose cells, human smooth muscle cells, human skeletal muscle cells, human leucocytes such as B-cells, T-cells, NK-cells or dendritic cells and stable, immortalized cell lines derived thereof (for example hTERT or oncogene immortalized cells). Appropriate culture media and conditions for the above described host cells are known in the art.

The host cells may e.g. be employed to produce large amounts of protein of interest, such as a binding molecule.

Exemplary binding proteins/binding molecules which are useful in the context of the present invention include, but are not limited to antibodies, antibody fragments such as Fab fragments, Fab′ fragments, F(ab′)2 fragments, single chain variable fragments (scFv), (single) domain antibodies, in particular those derived from camelids, llamas or sharks, isolated variable regions of antibodies (VL and/or VH regions), in particular those from humans or primates, CDRs, immunoglobulin domains, CDR-derived peptidomimetics, lectins, fibronectin domains, tenascin domains, protein A domains, SH3 domains, ankyrin repeat domains, and lipocalins or various types of scaffold-derived binding proteins as described.

The following relates to host cell proteins (HCP) that may be present in the starting composition, and, if present, are to be removed from (or the content reduced in) the composition comprising the protein of interest.

HCPs are proteins that are produced or encoded by cells or organisms that are used in the production process and are unrelated to the intended product. Some are necessary for growth, survival, and normal cellular processing whereas others may be non-essential. Like the intended product, HCPs may also be modified by the host with a number of post-translational modifications. Regardless of the utility, or lack thereof, HCPs are generally undesirable in a final drug substance. Though commonly present in small quantities (parts per million expressed as nanograms per milligrams of the intended protein) much effort and cost is expended by industry to remove them. Prior to the approval of a biological product for therapeutic use, the level of residual HCP in the product should be quantitatively measured. Thus, HCP must be typically eliminated or reduced and elimination or reduction must be demonstrated. Current analytical methods to assay for the presence of contaminant HCPs in recombinant biological products include SDS-PAGE, immunoblotting techniques and ELISA. There are many publications on the removal of HCP contamination and removal, for instance using for instance hydrophobic interaction chromatography (Shukla et al, Biotechnol. Prog. 2002, 18, 556-564), Protein A chromatography (Shukla et al, Biotechnol. Prog. 2008, 24, 11151121), or salt tolerant anion exchange ligands (Riordan et al, Biotechnol. Prog. 2009, Vol. 25, No. 6).

As shown herein, HCP content is however insufficiently removed by conventional purification methods. Even minor residual amounts can lead to long-term decreased storage stability. It is envisaged that the presence of the HCP in the composition can result in the occurrence of visible and/or sub-visible particles due to insoluble matter of surfactant degradants. Said particles may contravene the requirements for parenteral preparations (EP 2.9.19, EP 2.9.20, USP <787>, USP <788>, and USP <789>). Furthermore, these particles may comprise insoluble protein aggregates, which are more immunogenic than soluble protein. Accordingly, the herein provided composition/formulation comprising a protein of interest is a composition/formulation comprising a low content of HCP and/or preferably does not show visible particle formation, particularly during storage e.g. at 2-8° C. for at least 6 months, at least 12 months, at least 18 months, at least 24 months, at least 36 months or at least 60 months.

With visible particle formation is meant that particle(s) can be observed with the naked eye by a person skilled in the art, optionally using a means for magnification, such as a magnifying glass. In the regulatory world, there is a distinction between visible and non-visible particulate matter. Visible particulate is loosely defined as any particulate that can be detected with the unaided eye. Typically, visible objects are defined as objects that are 0.05 mm or larger. With the term “visible particles”, as used in the present invention, is meant: particles that are 0.05 mm or larger, preferably 0.1 mm or larger, more preferably 0.2 mm or larger, more preferably 0.5 mm or larger, most preferably 1 mm or larger. It is also possible to detect visible particle formation with equipment designed to detect such visible particles. The most common approach to automating the inspection for particulate in a clear solution, as is with the composition (obtained by a method) according to the invention, is to agitate the solution and image the solution over time. The imaging system generally consists of a machine vision camera, illumination (in this case backlighting) and a vision processor to analyze the images. Once the images have been acquired, they are then analyzed in sequence for image-to-image differences. The differences can be interpreted as objects moving inside the solution such as gas bubbles and particulate. In the case of larger, denser particulate, detection is achieved by filtering out the gas bubbles from the analysis, as they will rise up while the particulate sinks.

If the goal is to find particulate, smaller than about 1 mm in diameter, a more careful approach to agitation must be used to remove gas bubbles from the field of view. This can be done by agitating the solution through spinning, with careful attention to acceleration and deceleration rates. For the purpose of the present invention, however, the method of detection is not important for defining “visible particles” as long as the particles have a diameter as defined above.

A method according to the invention preferably results in a composition/formulation comprising less than 100 ppm, more preferably less than 10 ppm, more preferably less than 1 ppm, more preferably less than 0.1 ppm, most preferably less than 0.01 ppm HCPs, relative to the protein of interest content. For example, the composition/formulation comprises less than 100 ppm of (a) host cell protein(s), more preferably less than 50 ppm, more preferably less than 20 ppm, more preferably less than 10 ppm, more preferably less than 5 ppm, more preferably less than 2 ppm, most preferably less than 1 ppm or lower.

The method described herein is not limited to a particular protein/protein of interest. The protein/protein of interest in the starting composition may be any protein derived from any source.

Specifically, any therapeutic, diagnostic and/or pharmaceutical protein/protein of interest is contemplated as well as any protein of interest in scientific research. The following are non-limiting examples of proteins of interest:
interleukin receptor antagonist, interleukin-1 receptor antagonist like EBI-005 or anakinra, leptin, acetylcholinesterase, activated protein C (drotrecogin), activin receptor IIB antagonist, adenosine deaminase, agalsidase alfa, agonist of toll-like receptor 5 like entolimod, alpha-1 antitrypsin, alpha-1 proteinase inhibitor, alpha-galactosidase, alpha-human atrial natriuretic peptide, alpha-N-acetylglucosaminidase, alteplase, amediplase, amylin, amylin analogue, ANF-Rho, angiotensin (1-7), angiotensin II, angiotensin-converting-enzyme 2, anti-epithelial cell adhesion molecule single-chain antibody fragment, antithrombin alfa, antithrombin III, apoptosis inducing enzyme mi-APO, arginine deiminase, asparaginases like calaspargase, pegaspargase, crisantaspase, B domain deleted factor VIII like beroctocog alfa or octofactor, bectumomab (Lymphoscan), bile salt stimulated lipases like bucelipase alfa, binding protein directed against the respiratory syncytial virus like pavlizumab, bone morphogenetic proteins like BMP-2 (dibotermin alfa) or BMP-6, bouganin, bovine carboxyhemoglobin, bovine growth hormone, C1-Esterase-Inhibitor, C3 exoenzyme protein, carboxyhemoglobin, CD19 antagonist, CD20 antagonist like rituxan, CD3 receptor antagonist, CD40 antagonist, CD40L antagonist like dapirolizumab or Antova, cerebroside sulfatase, cethrin like VGX-210, chondroitin lyase, coagulation factor IX like nonacog gamma, conacog beta, albutrepenonacog alfa, coagulation factor VIIa like eptacog alfa, marzeptacog alfa, vatreptacog alfa, oreptacog alfa, coagulation factor VIII like susoctocog alfa, damoctocog alfa, turoctocog alfa, rurioctocog alfa, efinoroctocog alfa, efraloctocog alfa, simoctocog alfa, coagulation factor X, coagulation factor XIII like catridecacog, collagenase of Clostridium histolyticum, complement factor C3 inhibitor, complement receptor 5a antagonist, corticotrophin releasing factor, CSF1 receptor antagonists like FPA008, CSF1R antagonist, CTLA-4 antagonist like ipilimumab, cyanovirin-N, deoxyribonuclease I like dornase alfa, EGFR receptor antagonist, elastases like human type I pancreatic elastase like vonapanitase, endostatin, enkastim, epidermal growth factor, erythropoietin alfa, erythropoietin zeta, FcγIIB receptor antagonists, fibrinogenase, fibrinolytic enzyme like brinase, fibroblast growth factor 1 (human acidic fibroblast growth factor), fibroblast growth factor 18, fibroblast growth factor 2 (human basic fibroblast growth factor), fibroblast growth factor 21, fibroblast growth factor receptor 2 antagonists like FPA144, Fins-like tyrosine kinase 3 ligand, follicle-stimulating hormones like follitropin alfa or follitropin beta, fragment of human bactericidal/permeability-increasing protein 21 (opebacan/rBPI 21), gelonin, glucagon receptor agonist, glycoprotein IIb/IIIa antagonist like abciximab, glycosaminoglycan-degrading enzymes like condoliase, gp120/gp160, granulocyte colony stimulating factor (G-CSF), granulocyte macrophage colony stimulating factor (GM-CSF), heat-shock protein hsp 65 from Mycobacterium BCG fused with transcription factor E7 (verpasep caltespen), hepatocyte growth factor, hepatocyte growth factor receptor (HGFR) antagonist, hepcidin antagonist, Her2/neu receptor antagonist like herceptin, heterodimeric 15:IL-15Ra (hetIL-15), hirudin, hsp70 antagonist, human acid sphingomyelinase, human chorionic gonadotropin like choriogonadotropin alfa, human enzyme acid α-glucosidases like reveglucosidase alfa or alglucosidase alfa, human growth hormone, human keratinocyte growth factor (KGF), human matrix metalloproteinase, human myelin basic protein fragment, human osteogenic protein 1, human osteogenic protein-1, human parathyroid hormone, human thrombomodulin alpha, hyaluronidase like rHuPH20, hyaluronidases like human hyaluronidase PH-20 (vorhyaluronidase alfa), hyalosidase or bovhyaluronidase, hydrolytic lysosomal glucocerebroside-specific enzymes like glucocerebrosidase, velaglucerase alfa or taliglucerase alfa, iduronate-2-sulfatase, IgE antagonists like omalizumab, iIroquois homeobox protein 2 (IRX-2), insulin, insulin analog, integrin β4β1 antagonist, interferon tau, interferon-alpha, interferon-alpha antagonist, interferon-alpha superagonist, interferon-alpha-n3 (Alferon N Injection), interferon-beta, interferon-gamma, interferon-lambda, interleukin 2 fusion proteins like DAB(389)IL-2, interleukin-11 like oprelevkin, interleukin-12, interleukin-17 receptor antagonist, interleukin-18 binding protein, interleukin-2, interleukin-22, interleukin-4 like pitrakinra, interleukin-4 mutein, interleukin-6 receptor antagonist, interleukin-7, interleukin-22 receptor subunit alpha (IL-22ra) antagonist, irisin, islet neogenesis associated protein, kallidinogenase, lactoferrin, lactoferrin fragment, lanoteplase, lipase enzymes like burlulipase, rizolipase, epafipase or sebelipase alfa, luteinizing hormone, lutropin alpha, lymphocyte expansion molecule, lysostaphin, mammalian gastric lipase enzyme (merispace), mannosidases like velmanase alfa, melanocortin-4 receptor agonist, MEPE-derived 23-amino acid peptide, methionyl human stem cell factor (ancestim), microplasmin, N-acetylgalactosamine-6-sulfatase like elosulfase alfa, N-acetylglucosaminidase, nasaruplase beta, nerve growth factor, neuregulin-1, neurotoxin (e.g. a clostridial neurotoxin, like a Clostridium botulinum neurotoxin (such as Clostridium botulinum neurotoxin serotype A, B, C, D, E, F or G, particularly Clostridium botulinum neurotoxin serotype A), neutrophil gelatinase-associated lipocalin, ocriplasmin, Ornithodoros moubata complement inhibitor (OmCI/Coversin), osteoprotegerin, P128 (StaphTAME), pamiteplase, parathormone (PTH), PD-1 antagonist, PDGF antagonist, pentraxin-2 protein, phage lysin like HY133, phenylalanine ammonia lyase like valiase, phosphatases like tissue-nonspecific alkaline phosphatase or asfotase alfa, plasminogen, plasminogen variant like V10153, platelet derived growth factor-BB, porcine growth hormone, prohibitin-targeting peptide 1, proinsulin, protein A, protein C like drotrecognin, protein binding fibroblast growth factor receptor ligands like FP-1039, recombinant tissue factor pathway inhibitor (tifacogin), relaxin, relaxin analog like serelaxin, reteplase, rhPDGF-BB, ribonuclease like onconase or amphinase, senrebotase, serine protease inhibitors like conestat alfa, sfericase, sialidase, soluble complement receptor type 1, soluble DCC (deleted in colorectal cancer) receptor, soluble TACI receptor (atacicept), soluble tumor necrosis factor I receptor (sTNF-RI), soluble tumor necrosis factor II receptor (sTNF-RII), soluble VEGF receptor Flt-1, soluble, human FcγIIB receptor, staphylokinase, streptokinase, sulfamidase, T-cell receptor ligand, tenecteplase, thrombopoiesis-stimulating protein (AMG-531), thrombopoietin, thrombospondin-1, thyroid hormone, thyrotropin-releasing hormone (TRH) analog like taltirelin, tissue plasminogen activator, tissue-type plasminogen activator like pamiteplase, tripeptidyl peptidase I, tumor necrosis factor (TNFalpha), tumour necrosis factor α antagonist, uricase like rasburicase or pegadricase, urodilatin, urofollitropin, urokinase, uteroglobin, VEGF antagonist like ranbizumab or bevacizumab, VEGF/PDGF antagonist, VEGF/PDGF antagonist like a multi-VEGF/PDGF DARPin or a fusion protein, viscumin, von Willebrand factors like vonicog alfa. Interleukin receptor antagonist, especially interleukin-1 receptor antagonists, like EBI-005 or anakinra, and leptin, especially human leptin, or a mutant human leptin (huLeptin(W100Q), a human leptin mutant with a tryptophan to glutamine substitution at position 100 in the mature polypeptide chain),

Preferably, the protein/protein of interest is antibody. Accordingly, the invention relates in one aspect to a method comprising the steps of
(i) contacting a starting composition comprising a protein with a hydrolase inhibitor, wherein the hydrolase inhibitor is immobilized on a solid carrier;
(ii) recovering a composition comprising the protein,

wherein the protein is an antibody.

The term “antibody” is used herein in the broadest sense, encompasses various antibody structures and can be any molecule that can specifically or selectively bind to a target protein. An antibody may include or be an antibody or a domain/fragment thereof, wherein the domain/fragment shows substantially the same binding activity as the full-length antibody. Non-limiting examples are monoclonal antibodies, polyclonal antibodies, or multispecific antibodies (e.g., preferably bispecific antibodies). Preferably, the antibody is a monoclonal antibody or a bispecific antibody. Antibodies within the present invention may also be chimeric antibodies, recombinant antibodies, humanized antibodies or (fully-)human antibodies. Preferably, the antibody is a humanized or a human antibody. Examples of antibody fragments include but are not limited to Fv, Fab, Fab′, Fab′-SH, F(ab′)2.

Antibodies may also include multivalent molecules, multi-specific molecules (e.g., diabodies), fusion molecules, aptimers, avimers, or other naturally occurring or recombinantly created molecules. Illustrative antibodies useful in the present invention include antibody-like molecules. An antibody-like molecule is a molecule that can exhibit functions by binding to a target molecule (see for example Gill (2006) Curr Opin Biotechnol 17:653-658; Nygren (1997) Curr Opin Struct Biol 7:463-469; Hosse (2006) Protein Sci 15:14-27), and includes, for example, DARPins (WO 2002/020565), Affibodies (WO 1995/001937), Avimers (WO 2004/044011; WO 2005/040229), Adnectins (WO 2002/032925) and fynomers (WO 2013/135588).

Non-limiting examples of antibodies within the present invention are an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A antibody, an antiVEGF-A/ANG2 antibody, an anti-CD79b antibody, an anti-ST2 antibody, an anti-factor D antibody, an anti-factor IX antibody, an anti-factor X antibody, an anti-abeta antibody, an antitau antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, a FAP-IL2v fusion protein, ocrelizumab, pertuzumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, lebrikizumab, omalizumab ranibizumab, emicizumab, selicrelumab, prasinezumab, glofitamab, simlukafusp alfa, and RG7827.

Preferred herein are anti-CD20/anti-CD3 antibodies (in particular anti-CD20/anti-CD3 bispecific antibodies, especially glofitamab), anti-Her2 antibodies (in particular trastuzumab), and/or anti-IL6-receptor antibodies (in particular tocilizumab).

When the method of the invention described herein is used for purification of Trastuzumab it is envisaged that step “(i) contacting a starting composition comprising a protein with a hydrolase inhibitor, wherein the hydrolase inhibitor is immobilized on a solid carrier” is performed after the Trastuzumab solution was subjected to Protein A chromatography. Glofitamab is an anti-CD20/anti-CD3 bispecific antibody and is also known as RG6026. The amino acid sequence of Glofitamab is disclosed in PCT/EP2015/067776 (WO/2016/020309). When the method of the invention described herein is used for purification of Glofitamab it is envisaged that step “(i) contacting a starting composition comprising a protein with a hydrolase inhibitor, wherein the hydrolase inhibitor is immobilized on a solid carrier” is performed before the Glofitamab solution is subjected to mixed mode anion exchange chromatography (MMAEX), i.e. the conditioned Protein A Eluate. In context of Glofitamab purification the term MMAEX load solution refers to the antibody solution before it is subjected to NMAEX chromatography

In one preferred aspect, the protein of interest is tocilizumab, the inhibitor is Orlistat B, the agent (linker) comprising an azido group and an biotin group separated by a linker for coupling to a solid carrier is azobiotin-azide, and the solid carrier is streptavidin Mag Sepharose (beads) (cf. Example 1 and 2).

In one preferred aspect, the protein of interest is Glofitimab, the inhibitor is Orlistat B, the agent (linker) comprising an azido group and an biotin group for coupling to a solid carrier is biotin-PEG4-azide and the solid carrier is Streptavidin Sepharose® High Performance bead (cf. Example 3).

In one preferred aspect, the protein of interest is Glofitimab, the inhibitor is Orlistat B, the agent (linker) comprising an azido group and an amino group for coupling to a solid carrier is azido-PEG4-amine, and the solid carrier is NHS-activated Sepharose® 4 Fast Flow beads (cf. Example 3).

In one preferred aspect, the protein of interest is Trastuzumab, the inhibitor is Orlistat A or Orlistat B, the agent (linker) comprising an azido group and an biotin group for coupling to a solid carrier is biotin-PEG4-azide and the solid carrier is Streptavidin Sepharose® High Performance bead (cf. Example 4).

In one preferred aspect, the protein of interest is Trastuzumab, the inhibitor is Orlistat A or Orlistat B, the agent (linker) comprising an azido group and an amino group for coupling to a solid carrier is azido-PEG4-amine, and the solid carrier is NHS-activated Sepharose® 4 Fast Flow beads (cf. Example 4).

The agents/linkers mentioned above are known as Biotin-PEG4-Azide (CAS 1309649-57-7), Azo-Biotin-Azide (CAS 1339202-33-3) (also known as Azo-Biotin-PEG3-Azide) and Azido-PEG4-Amine (CAS 951671-92-4).

Tocilizumab is an anti-IL6-receptor antibody. The CAS Registry Number of Tocilizumab is 375823-41-9. The INN Tocilizumab is disclosed in WHO Drug Information, Vol. 18, No. 3, 2004.

The term “protein” (protein-of-interest) and particularly “antibody” as used herein includes and/or relates to biosimilar thereof. Thus, the term “protein” (protein-of-interest) and particularly “antibody” as used herein can be understood to refer to “protein and/or a biosimilar thereof” (or likewise “protein-of-interest and/or a biosimilar thereof”), “antibody and/or a biosimilar thereof”, respectively, and so on.

A biosimilar is a biologic medical product (also known as biologic) highly similar to another already approved biological medicine (the ‘reference medicine’). The “‘reference medicine” as used herein also refers to a “reference protein/reference protein-of-interest” as defined herein, in particular if that “reference protein/reference protein-of-interest” is approved (by the authorities such as EMA, FDA and the like).

Biosimilars are approved according to the same standards of pharmaceutical quality, safety and efficacy that apply to all biological medicines. The term “biosimilar” as used herein refers in general to a protein/protein-of-interest that has the same, i.e. identical, (or substantially the same) amino acid sequence as a reference protein/protein-of-interest as defined herein. It is understood that a “biosimilar” can have different glycosylation or other characteristics than the reference protein/protein-of-interest.

Exemplary reference protein/protein-of-interests are described herein, e.g. ocrelizumab, pertuzumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, lebrikizumab, omalizumab ranibizumab, emicizumab, selicrelumab, prasinezumab, glofitamab, simlukafusp alfa, and RG7827. Preferred herein are trastuzumab, tocilizumab, and glofitamab.

The amino acid sequences of the reference protein/protein-of-interests are easily identifiable via the INN and related publications.

In accordance with the above, if a specific reference protein (reference protein-of-interest) is mentioned herein, the specific reference protein (reference protein-of-interest) includes and relates to a biosimilar thereof (preferably wherein the biosimilar is a protein that has the same amino acid sequence as the reference protein-of-interest).

For example, the term “anti-CD20/anti-CD3 antibody” can refer to “anti-CD20/anti-CD3 antibody and/or a biosimilar thereof” (preferably wherein the biosimilar is an anti-CD20/anti-CD3 antibody that has the same amino acid sequence as the reference anti-CD20/anti-CD3 antibody).

For example, the term “anti-CD20/anti-CD3 bispecific antibody” can refer to “anti-CD20/anti-CD3 bispecific antibody and/or a biosimilar thereof” (preferably wherein the biosimilar is an anti-CD20/anti-CD3 bispecific antibody that has the same amino acid sequence as the reference anti-CD20/anti-CD3 bispecific antibody).

For example, the term “glofitamab” can refer to “glofitamab and/or a biosimilar thereof”. (preferably wherein the biosimilar is an antibody that has the same amino acid sequence as the reference antibody glofitamab).

For example, the term “anti-Her2 antibody” can refer to “anti-Her2 antibody and/or a biosimilar thereof” (preferably wherein the biosimilar is an anti-Her2 antibody that has the same amino acid sequence as the reference anti-Her2 antibody).

For example, the term “trastuzumab” can refer to “trastuzumab and/or a biosimilar thereof” (preferably wherein the biosimilar is an antibody that has the same amino acid sequence as the reference antibody trastuzumab).

For example, the term “anti-IL6-receptor antibody” can refer to “anti-IL6-receptor antibody and/or a biosimilar thereof” (preferably wherein the biosimilar is an anti-IL6-receptor antibody that has the same amino acid sequence as the reference anti-IL6-receptor antibody).

For example, the term “tocilizumab” can refer to “tocilizumab and/or a biosimilar thereof” (preferably wherein the biosimilar is an antibody that has the same amino acid sequence as the reference antibody tocilizumab).

The above explanations apply, mutatis mutandis, to all proteins/protein-of-interest, specifically antibodies, disclosed herein.

The invention further relates to a method for preparing a protein formulation. Said method for preparing a protein formulation may comprise the method as described herein above and further the addition of a surfactant, preferably a fatty acid ester, to the composition comprising the protein. In other words, the method for preparing a protein formulation may comprise supplementing the composition comprising the protein as prepared by the method described herein above with a surfactant, preferably a fatty acid ester.

As shown herein above, the provided method can be used to prepare a protein composition with a reduced content of impurities, such as host cell proteins, in particular a hydrolase and/or with reduced hydrolytic activity. Protein compositions prepared in accordance with the invention can thus advantageously be used to prepare a protein formulation, the latter showing improved (physical) stability even if, for example, substrates of such host cell proteins, like surfactants are present in the protein formulation. Preferably reduced particle formation, preferably no visible particle formation, occurs consequently during long-term storage Accordingly, the invention relates in one aspect to a method for preparing a protein formulation comprising the steps of the method as described herein above, wherein the method for preparing the protein formulation further comprises adding a surfactant, preferably a fatty acid ester to the composition comprising the protein.

In one embodiment the method for preparing a protein formulation may comprise step
(i) contacting a starting composition comprising a protein with a hydrolase inhibitor, wherein the hydrolase inhibitor is immobilized on a solid carrier;
(ii) recovering a composition comprising the protein,

and further adding a surfactant, preferably a fatty acid ester to the composition comprising the protein.

The invention further relates in one aspect to a method for preparing a protein formulation, wherein the method for preparing the protein formulation further comprises adding a surfactant, preferably a fatty acid ester, to the composition comprising the protein obtained by or obtainable by the method as described herein above.

Surfactants are useful as cosolvents and stabilizers. They function by associating with both a hydrophilic surface and a lipophilic surface to maintain dispersion and structural stability of ingredients, like proteins. Surfactants are added to protein formulations primarily to enhance protein stability against mechanical stress, such as air/liquid interface and solid/liquid interface shear. The term “surfactant” as used herein refers in particular to “fatty acid esters” as explained and defined herein.

When the surfactants are degraded the surfactant break down products may form visible or subvisible particles. It is also envisaged that with decreasing surfactant concentrations in protein formulations, proteins may in some cases become structurally unstable in solution, and form multimeric aggregates that eventually become visible or subvisible particles.

The term “fatty acid ester” is used herein in the broadest sense and refers to any organic compound that contains a fatty acid chain linked to a head group via an ester bond. An ester bond is formed when a hydroxyl group (e.g., an alcohol or carboxylic acid) is replaced by an alkoxy group. Usually, esters are derived from a carboxylic acid and an alcohol. The alcohol group is generally referred to as the head group. The alcohol may be glycerol, sorbitol, sorbitan, iso-sorbide or the like. In case of polysorbates the esterification takes place between the carboxylic acid and the terminal hydroxyl group of the polyoxyethylene repeating units attached to e.g. sorbitol.

The term “fatty acid” or “fatty acid chain” means a carboxylic acid having an aliphatic tail. An aliphatic tail is simply a hydrocarbon chain comprising carbon and hydrogen, and in some cases, oxygen, sulphur, nitrogen and/or chlorine substitutions.

Aliphatic tails can be saturated (as in saturated fatty acids), which means that all carbon-carbon bonds are single bonds (i.e., alkanes). Aliphatic tails can be unsaturated (as in unsaturated fatty acids), wherein one or more carbon-carbon bonds are double bonds (alkenes), or triple bonds (alkynes).

Fatty acids are generally designated as short-chain fatty acids, which have fewer than six carbons in their aliphatic tails, medium-chain fatty acids having six to twelve carbons, long-chain fatty acids having thirteen to twenty one carbons, and very long chain fatty acids having aliphatic tails of twenty two carbons and longer. As mentioned above, fatty acids are also categorized according to their degree of saturation, which correlates to stiffness and melting point. Common fatty acids include caprylic acid (8 carbons: 0 double bonds; 8:0), capric acid (10:0), lauric acid (12:0), myristic acid (14:0), myristoleic acid (14:1), palmitic acid (16:0), palmitoleic acid (16:1), sapienic acid (16:1), stearic acid (18:0), oleic acid (18:1), elaidic acid (18:1), vaccenic acid (18:1), linoleic acid (18:2), linelaedic acid (18:2), alpha-linolenic acid (18:3), arachidic acid (20:0), arachidonic acid (20:4), eicosapentaenoic acid (20:5), behenic acid (22:0), erucic acid (22: 1), docosahexaenoic acid (22:6), lignoceric acid (24:0), and cerotic acid (26:0).

Preferably, the fatty acid ester is a polyoxyethylene sorbitan or iso-sorbide fatty acid mono-, di- or triester. In a preferred embodiment, the fatty acid ester within the invention is a monoester of polyoxyethylene (4) sorbitan, a monoester of polyoxyethylene (5) sorbitan or a monoester of polyoxyethylene (20) sorbitan.

A monoester of polyoxyethylene (4) sorbitan, of polyoxyethylene (5) sorbitan or of polyoxyethylene (20) sorbitan is preferably selected from the group consisting of Polysorbate 20, Polysorbate 40, Polysorbate 60, Polysorbate 61, Polysorbate 80, Polysorbate 81, Polysorbate 120 and combinations thereof. Polysorbate 20 mostly comprises the monolaurate ester of polyoxyethylene (20) sorbitan. Polysorbate 40 mostly comprises the monopalmitate ester of polyoxyethylene (20) sorbitan. Polysorbate 60 mostly comprises the monostearate ester of polyoxyethylene (20) sorbitan. Polysorbate 61 mostly comprises the monostearate ester of polyoxyethylene (4) sorbitan. Polysorbate 80 mostly comprises the monooleate ester of polyoxyethylene (20) sorbitan. Polysorbate 81 mostly comprises the monooleate ester of polyoxyethylene (5) sorbitan. Polysorbate 120 mostly comprises the monoisostearat ester of polyoxyethylene (20) sorbitan.

In another preferred embodiment, the fatty acid ester is a monoester of polyoxyethylene (20) sorbitan. Preferably, a monoester of polyoxyethylene (20) sorbitan is selected from the group consisting of Polysorbate 20, Polysorbate 40, Polysorbate 60, Polysorbate 80, Polysorbate 120 and combinations thereof. More preferably, a monoester of polyoxyethylene (20) sorbitan is selected from the group consisting of Polysorbate 20, Polysorbate 40, Polysorbate 60, Polysorbate 80 and combinations thereof. Most preferably, a monoester of polyoxyethylene (20) sorbitan is selected from the group consisting of Polysorbate 20, Polysorbate 80 and combinations thereof.

In another preferred embodiment the fatty acid ester within the invention is a triester of polyoxyethylene (20) sorbitan. Preferably, a triester of polyoxyethylene (20) sorbitan is selected from the group consisting of Polysorbate 65, Polysorbate 85 and combinations thereof. Polysorbate 65 mostly comprises the tristearat ester of polyoxyethylene (20) sorbitan. Polysorbate 85 mostly comprises the trioleat ester of polyoxyethylene (20) sorbitan.

In another preferred embodiment the fatty acid ester within the invention is Mono- or Di-acylglycerol, saccaride-fatty acid ester or α-tocopheryl PEG succinate (TPGS). It is clear that all surfactants mentioned herein can be combined.

As mentioned above the degradation of surfactants may be the hydrolysis of fatty acid esters. The loss of surfactants through hydrolysis may lead to aggregation of proteins, which may lead to the formation of visible and/or subvisible particle formation. Also the degradation products of surfactants itself may lead to the formation of visible and/or subvisible particles. As envisaged herein and described above the inventive method may reduce the hydrolytic activity in the composition comprising the protein. Accordingly, it is also envisaged that formulations prepared from said composition comprising the protein may have a reduced hydrolytic activity or are essentially free of hydrolytic activity. The skilled person will acknowledge that reduced or no hydrolytic activity in the prepared formulation will lead to a reduced amount of visible and/or subvisible particles in the prepared formulation. It is also possible that the prepared formulation will be free or essentially free of visible and/or subvisible particles. It is especially important that the prepared formulation will be free or essentially free of visible and/or subvisible particle upon storage. Corresponding definitions have already been provided hereinabove. These definitions and explanations apply here mutatis mutandis.

Accordingly, the invention relates in one aspect to a method for preparing a protein formulation as described herein, wherein the protein formulation is (essentially) free of visible and/or subvisible particles, in particular upon storage.

Visible and subvisible particles are characterized by the analytical methods (see Table A for a list of the methods) and classified in compliance with the requirements within the corresponding regulations (EP 2.9.19, EP 2.9.20, USP <787>, USP <788>, and USP <789>). The quantification and chemical identification of the particles is used to inform the particle risk assessment. Visible particle detection by visual inspection is probabilistic and dependent on particle size, morphology, color, density, reflectivity, the properties of the surrounding formulation, the measurement conditions, and human performance. Published studies indicate that 150 μm is the size of spherical polystyrene particle standards (used to calibrate the method) that is detected with >70% probability by human inspectors as discussed in USP <1790> (Melchore (2011) AAPS PharmSciTech. 12: 215-221).

Subvisible particles are also evaluated in compliance with the pharmacopoeia and product specification limits for particles ≥10 m and ≥25 μm (and ≥50 μm for ophthalmic solutions). The particle population ≥2 μm and ≥5 m are usually monitored as well, especially during product development. The upper limit for the detection of subvisible particles based on the currently available technologies (e.g., HIAC) is 100 μm.

In alignment with the United States Pharmacopeia (USP) (USP <788>), the “visible gray zone” refers to the size range at the lower end of visible inspection, where the probability of particle detection by visible inspection falls from <70% at 150 μm to zero near 50 μm, which is the upper end of the subvisible particles detection capability.

The allowable amount of particles may be less than 7, less than 6, less than 5, less than 4, less than 3, less than 2, or less than 1 particles per 10 containers.

The skilled person is well aware how to receive additional information on particles (e.g. European Pharmacopeia (EP) 2.9.19, EP 2.9.20, USP <787>, USP <788>, USP <789>, Mathonet (2016) PDA J Pharm Sci Technol. 70: 392-408, Rech (2020) J. Pharm. Sci. 109: 1725-1735)

TABLE A
Methods for detecting/characterization of particles

| | | |
|---|---|---|
| Method | Purpose | Method Description/Reference |

Particle-related

| | | |
|---|---|---|
| Visible Particles | QC | Visible particles are tested using the compendial method |
| | | during QC in compliance with the requirements within the |
| | | USP, Ph. Eur., and JP. Each particle finding will be further |
| | | characterized using extended characterization methods, |
| | | such as FTIR. |
| Subvisible Particles | QC | Subvisible particles ≥10 μm and ≥25 μm (and ≥50 μm |
| | | for intravitreal injection) are measured with the |
| | | compendial light obscuration method for release and |
| | | stability testing. The subvisible particles acceptance |
| | | cnteria are set in compliance with the requirements within |
| | | the USP, Ph. Eur., and JP. Subvisible particles ≥2 μm |
| | | and ≥5 μm are usually monitored as well, especially |
| | | during development. |
| FTIR | Characterization | FTIR is used for extended characterization of the nature |
| | | of particles. Particles are isolated from the drug product |
| | | container and immobilized on a gold filter. During this |
| | | procedure, an infrared spectrum is obtained and |
| | | compared to a spectra library. This method can |
| | | differentiate particles composed of protein, silicone, or |
| | | fatty acids from other particulate types (e.g., extrinsic |
| | | particles according to USP <1787>). |
| Flow Imaging | Characterization | Flow imaging (also known as dynamic imaging particle |
| | | analysis) is used as an extended characterization method |
| | | and may be applied to characterize particle appearance |
| | | and size distribution. Flow imaging technology captures |
| | | digital microscope images of particles in solution as they |
| | | pass through a flow cell. Each individual particle is |
| | | measured using standard image analysis algorithms. The |
| | | system provides various morphological descriptors and |
| | | actual pictures of the particles. |

As mentioned already above authorities provide limitations on the number of visible and/or subvisible particles for pharmaceutical formulations. Consequently, pharmaceutical formulations can no longer be used after a certain amount of time due to the formation of visible and/or subvisible particles.

It is envisaged herein that a protein formulation with reduced formation of visible and/or subvisible particle has an increased stability or is longer stable. In other words, the protein formulation with reduced formation of visible and/or subvisible particle has an increased shelf life.

Accordingly, a protein formulation with reduced hydrolytic activity may have an increased stability, an increased shelf life or is longer stable.

Accordingly, the invention relates in one aspect to a method for preparing a protein formulation comprising the steps of the method as described herein, wherein the method for preparing the protein formulation further comprises adding a surfactant, preferably a fatty acid ester to the composition comprising the protein, wherein the protein formulation has an increased stability, an increased shelf life and/or is (longer) stable, particularly storage stable.

Long-term stability data of a drug product (e.g. protein formulation as defined herein) must be generated to confirm adequate stability throughout shelf-life. Three standard long-term conditions are typically considered for biotechnological drug products. The long-term condition is also named recommended storage condition. It represents the proposed label storage condition.

Standard long-term conditions can include
i) storage in a freezer (−20° C.) (according to ICH Q1A) for a minimum time period of 6 months (according to ICH Q5C),
ii) storage in a refrigerator (5° C. (according to ICH Q1A), i.e. recommended storage condition for Drug product) for a minimum time period of 6 months (according to ICH Q5C),
iii) storage at room temperature (25° C.) (according to ICH Q1A) for a minimum time period of 6 months (according to ICH Q5C).

Standard long-term conditions (also termed “standard long term storage condition” or “recommended storage condition”) can include storage for at least 6 months, at least 12 months, at least 18 months, at least 24 months, at least 36 months or at least 60 months.

Thus, stability of a drug product (e.g. protein formulation as defined herein) can be tested and/or confirmed under such “standard long-term conditions”.

It is understood that in practice a shelf-life (i.e. stability) of at least 18 months is required for approval of a commercial product. Thus, standard long-term conditions (also termed “standard long term storage condition” or “recommended storage condition”) to be used herein preferably include storage for at least 18 months. However, for practical purposes long-term stability studies for at least 6 months may be sufficient, as the data can be extrapolated to a longer storage time.

It is understood that the term “at least X months” can refer to the specific time period i.e. “X months”. For example, the term “(storage) for at least 6 months” can refer to “(storage) for 6 months”, and so on.

Critical quality attributes should be evaluated periodically applying appropriate analytical methodology.

As described herein above it is envisaged that step (i) of the inventive method reduces hydrolytic activity in the composition comprising the protein.

Accordingly, the invention relates in one aspect to a method for preparing a protein formulation as described herein, wherein the protein formulation has an increased stability, an increased shelf-life or is longer stable compared to a protein formulation not subjected to step (i) of the method.

The invention further relates to a method for preparing a protein formulation as described herein, wherein the protein formulation can be stored for up to 18, up to 24, up to 36, or up to 60 months at 4° C., in particular wherein the formulation is (more) stable, in particular (more) storage stable.

For example, the formulation does not show substantial visible particle formation during stability testing, preferably under standard long-term conditions as described herein, for example at 2-8° C., preferably at about 5° C. for at least 2 months, more preferably for at least 3 months, for at least 6 months, at least 12 months, at least 18 months, at least 24 months, at least 36 months or at least 60 months.

With no substantial visible particle formation is meant that the <20 visible particles are formed in 1 mL of formulation. Preferably, 15 or less particles, more preferably 10 or less, more preferably 5 or less, most preferably less than 1 particles per 1 mL formulation are formed during stability testing at the conditions specified for storage of the formulation.

As described above, the term “visible particle” as used herein denotes particles that are 50 μm in diameter or more, preferably 100 μm or more, more preferably 500 μm or more, most preferably 1 mm or more. The particles can be either observed by the naked eye, optionally using magnifying means or by an automated process, such as for instance a film camera and suitable means for analyzing the film material, as described before.

As described above it is envisaged that the hydrolytic activity in the prepared protein formulation is reduced. Consequently, also the degradation of the fatty acid ester may be reduced in the prepared protein formulation.

Accordingly, the invention relates to a method for preparing a protein formulation as described herein, wherein the degradation of the fatty acid ester is less than 20%, less than 15%, less than 10%, less than 9%, less than 8%, less than 7%, less than 6%, less than 5%, less than 4%, less than 3%, less than 2% or less than 1% within preferably 24 months for example, compared to the initial formulation (formulation prior to the start of storage). This can be determined during stability testing (preferably under standard long-term conditions as described herein).

Preferably, the degradation of the fatty acid ester is less than 10% within 24 months, for example, compared to the initial formulation (formulation prior to the start of storage). This can be determined during stability testing (preferably under standard long-term conditions as described herein).

As mentioned above the protein in the composition comprising a protein is preferably an antibody. Accordingly, it is also envisaged herein that the protein in the protein formulation within the invention is an antibody. The concentration of the antibody in the formulation may be lower than 1 mg/ml or higher than 250 mg/ml. Usually the concentration of the antibody will be between 1 mg/ml and 250 mg/ml.

Accordingly, the invention relates to a method for preparing a protein formulation, wherein the protein is and antibody and wherein the antibody concentration is at least 1 mg/ml and up to 250 mg/ml, preferably at least 5 mg/ml and up to 200 mg/ml.

For example, the antibody concentration may be 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml or 200 mg/ml.

Although clear for the skilled person it is noted that besides a surfactant additional substances may be added to the composition comprising the protein. Such substances may be buffers, excipients, diluents, stabilizers, carriers and combinations thereof.

Accordingly, the invention relates to a method for preparing a protein formulation as described herein further comprising adding a buffer, excipient, diluent, stabilizer and/or carrier to the composition comprising the protein.

Examples of suitable buffers, excipients, diluents, stabilizers and carriers are well known in the art. Suitable buffers, excipients, diluents, stabilizers and carriers may include any material as long as the protein in the protein formulation retains its biological activity upon contact with said buffers, excipients, diluents, stabilizers and carriers.

Non-limiting examples of buffers are provided herein above. Non-limiting examples for the other substances are trehalose, sucrose, sorbitol, sodium chloride, potassium chloride, and calcium chloride, methyl p-hydroxybenzoate, ethyl p-hydroxybenzoate, sorbic acid, phenol, cresol, chlorocresol, human serum albumin, lecithin, dextran, ethyleneoxide-propyleneoxide copolymer, hydroxypropyl cellulose, methylcellulose, polyoxyethylene hydrogenated castor oil, polyethylene glycol, N-acetylcysteine, N-acetylhomocysteine, thioctic acid, thiodiglycol, thioethanolamine, thioglycerol, thiosorbitol, thioglycolic acid and salts thereof, sodium thiosulfate, glutathione, erythorbic acid, dibutylhydroxytoluene, butylhydroxyanisole, tocopherol, tocopherol acetate, L-ascorbic acid and salts thereof, L-ascorbyl palmitate, L-ascorbyl stearate, sodium hydrogen sulfite, sodium sulfite, triamyl gallate, and propyl gallate, disodium ethylenediamine tetraacetate (EDTA), sodium pyrophosphate and sodium metaphosphate.

It is clear that the prepared protein formulation may be a pharmaceutical composition (or in other words a pharmaceutical formulation).

Accordingly, the invention relates to a method for preparing a protein formulation as described herein, wherein the protein formulation is a pharmaceutical composition.

Administration of the pharmaceutical compositions as described herein may be effected by different ways, e.g., by parenteral, subcutaneous, intravenous, intraarterial, intraperitoneal, topical, intrabronchial, intrapulmonary and intranasal administration and, if desired for local treatment, intralesional administration. The pharmaceutical compositions as described herein may also be administered directly to the target site, e.g., by biolistic delivery to an external or internal target site, like a specifically effected organ.

It is also envisaged herein that the protein formulation can be a diagnostic composition, e.g. for in vivo or in vitro/ex vivo diagnostic methods.

An advantage of the invention is that the hydrolase inhibitor is immobilized on a solid carrier and does not contaminate the composition comprising the protein and the protein formulation derived therefrom.

Accordingly, the invention relates to a composition comprising a protein and/or to a protein formulation, wherein the composition comprising the protein and/or the protein formulation are essentially free of hydrolase inhibitors (e.g. hydrolase inhibitors are not present (or their presence is not detectable) in the composition comprising the protein and/or the protein formulation or hydrolase inhibitors are present only in minute amounts).

The invention further relates to a composition comprising a protein obtained or obtainable by the method described herein.

Accordingly, the invention also relates to a protein formulation obtained or obtainable by the method for preparing a protein formulation as described herein.

It is also envisaged herein that the protein formulation of the invention is used as a medicament.

In other words, it is envisaged that the protein formulation of the invention is used as a medicament or is used in medicine.

Accordingly, the present invention further relates to a protein formulation obtained or obtainable by the method for preparing a protein formulation as described herein, wherein the protein formulation is for use as a medicament. Accordingly, the present invention further relates to a protein formulation obtained or obtainable by the method for preparing a protein formulation as described herein, wherein the protein formulation is for use in medicine. In addition, the present invention further relates to a protein formulation obtained or obtainable by the method for preparing a protein formulation as described herein, wherein the protein formulation is for use in the treatment of a disease. The present invention also relates to a protein formulation obtained or obtainable by the method for preparing a protein formulation as described herein, wherein the protein formulation is for use in the preparation of a medicament for the treatment of a disease or is for use in the preparation of a diagnostic composition.

Furthermore, the invention relates to a method of treatment of a disease comprising administering to a subject in need thereof a protein formulation obtained or obtainable by the method for preparing a protein formulation as described herein.

It is clear that the hydrolase inhibitor immobilized on a solid carrier can be used for preparing a composition comprising a protein and/or a protein formulation.

Thus, in one aspect, the invention relates to the use of a hydrolase inhibitor immobilized on a solid carrier for preparing a composition comprising a protein. Accordingly, in one aspect, the invention also relates to the use of a hydrolase inhibitor immobilized on a solid carrier for preparing a protein formulation. In one aspect, the invention also relates to the use of a hydrolase inhibitor immobilized on a solid carrier for preparing a protein composition and/or a protein formulation. In one aspect, the invention also relates to the use of a hydrolase inhibitor immobilized on a solid carrier for removing or reducing hydrolytic activity in a protein composition and/or in a protein formulation. In one aspect, the invention also relates to the use of a hydrolase inhibitor immobilized on a solid carrier for removing or reducing (content of) impurities, particularly of host cell proteins, and preferably of (a) hydrolase(s) in a protein composition and/or in a protein formulation. In one aspect, the invention also relates to the use of a hydrolase inhibitor immobilized on a solid carrier for inhibiting or reducing (the formation of) visible and/or sub-visible particles and/or the occurrence/presence of surfactant degradants in a protein composition and/or in a protein formulation.

As used herein, the terms “comprising”, “including”, “having” or grammatical variants thereof are to be taken as specifying the stated features, integers, steps or components but do not preclude the addition of one or more additional features, integers, steps, components or groups thereof. The terms “comprising”/“including”/“having” encompass the terms “consisting of” and “consisting essentially of”. Thus, whenever the terms “comprising”/“including”/“having” are used herein, they can be replaced by “consisting essentially of” or, preferably, by “consisting of”.

The terms “comprising”/“including”/“having” mean that any further component (or likewise features, integers, steps and the like) can be present.

The term “consisting of” means that no further component (or likewise features, integers, steps and the like) can be present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed product, composition, device or method and the like.

Thus, the term “consisting essentially of” means that specific further components (or likewise features, integers, steps and the like) can be present, namely those not materially affecting the essential characteristics of the product, composition, device or method. In other words, the term “consisting essentially of” (which can be interchangeably used herein with the term “comprising substantially”), allows the presence of other components in the product, composition, device or method in addition to the mandatory components (or likewise features, integers, steps and the like), provided that the essential characteristics of the product, composition, device or method are not materially affected by the presence of other components.

As used herein the term “about” refers to +10%.

As used herein, “a” or “an” may mean one or more.

The present invention is illustrated by the appended non-limiting Figures and Examples.

### BRIEF DESCRIPTION OF FIGURES

FIG. 1: Chemical structures of hydrolase inhibitors used for immobilization

A) Alkyne-substituted Orlistat variant 1 “Orlistat A” as used in example 4 (formula (1) or likewise compound A) B) Alkyne-substituted Orlistat variant 2 “Orlistat B” as used in examples 1-4. (formula (2) or likewise compound B) C) “Bis-enol-ester” reagent as used in example 5 (formula (3) or likewise compound C).

FIG. 2: Removal of recombinant LPLA2 from a Tocilizumab solution using Orlistat B immobilized on Streptavidin Mag Sepharose beads.

Orlistat B: Mag-Sepharose-Streptavidin+Azo-biotin-azide (Azo-Biotin-PEG3-Azide)+Alkyne-Orlistat B;

beads control: Mag-Sepharose-Streptavidin+Azo-Biotin-Azide;

Fishing in 5 mg/mL Tocilizumab+50 ppm rec. LPLA2 (=250 ng/mL LPLA2).

A) Lipase activity assay of LPLA2-spiked Tocilizumab supernatants after incubation with conjugated beads. Activities are given for the equivalent 5 ng/mL LPLA2.

B) Spectral counts of LPLA2 LC MS/MS analysis from on-bead digested samples.

FIG. 3: Lipase activity assay of Tocilizumab solution after fishing with conjugated beads in 100 mg/mL Tocilizumab+50 ppm rec. LPLA2 (=5 μg/mL LPLA2).

Activities are given for the equivalent of 5 ng/mL LPLA2.

Orlistat B: Mag-Sepharose-Streptavidin+Azo-Biotin-Azide+Alkyne-Orlistat B;

linker control: Mag-Sepharose-Streptavidin+Azo-Biotin-Azide;

mAb+LPLA2: 100 g/l Tocilizumab+50 ppm rec. LPLA2 (=5 μg/mL LPLA2);

mAb: 100 g/L Tocilizumab.

FIG. 4: Lipase activity assay for glofitamab MMAEX load solution

Given activities are normalized to 100 μg protein. Each bar represents one technical fishing replicate. The bead compositions for fishing experiments in glofitamab MMAEX load solution were as follows:

NHS-activated Sepharose

“NHS w/ Orlistat B”: NHS-activated Sepharose beads+Azido-PEG4-Amine+Alkyne-Orlistat B;

“NHS w/o Orlistat B”: NHS-activated Sepharose beads+Azido-PEG4-Amine+Biotin-PEG4-Alkyne;

“NHS”: NHS activated Sepharose beads

Steptavidin Sepharose beads

“Seph-SA w/ Orlistat B”: Streptavidin Sepharose® High Performance beads+Biotin-PEG4-Azide+Alkyne-Orlistat B;

“Seph-SA w/o Orlistat B”: Streptavidin Sepharose® High Performance beads+Biotin-PEG4-Azide+Biotin-PEG4-Alkyne;

“Seph-SA”: Streptavidin Sepharose® High Performance beads

“Glofitamab ctrl.” refers to the activity of the starting protein composition

Available bead material was just enough to perform duplicate fishing experiments for “NHS”, “SA w/Orlistat B” and “SA”, therefore the triplicate measurement is missing.

FIG. 5: Lipase activity assay for Conditioned protein A elution pool samples of Trastuzumab. Load only, without any beads. Given activities are normalized to 100 μg protein. The bead compositions for the experiments in Conditioned protein A elution pool of Trastuzumab were as follows:

Seph-NHS w/ OrlistatA: NHS-activated Sepharose beads+Azido-PEG4-Amine+Alkyne-Orlistat A;

Seph-NHS w/ OrlistatB: NHS-activated Sepharose beads+Azido-PEG4-Amine+Alkyne-Orlistat B;

Seph-NHS w/o Orlistat: NHS-activated Sepharose beads+Azido-PEG4-Amine+Biotin-PEG4-Alkyne;

Seph-SA w/ Orlistat A: Streptavidin Sepharose® High Performance beads+Biotin-PEG4-Azide+Alkyne-Orlistat A;

Seph-SA w/ Orlistat B: Streptavidin Sepharose® High Performance beads+Biotin-PEG4-Azide+Alkyne-Orlistat B;

Seph-SA w/o Orlistat: Streptavidin Sepharose® High Performance beads+Biotin-PEG4-Azide+Biotin-PEG4-Alkyne;

Trastuzumab ctrl. refers to the activity of the starting protein composition.

FIG. 6: Peptide peak intensities of lipase CalB2 bound on bis-enol ester functionalized beads.

Given are the LC-MS based read out's in peak intensity (right) normalized level for covalently bound Lipase CalB2 (based on two peptides (Peptide CalB2 Lipase 1: LMAFAPDYK and CalB2 Lipase 2: PFAVGK) on functionalized beads after washing steps supported by the bis-enol-ester moiety (compound C (or likewise formula (3)) after click reaction with bead surface exposed alkyne groups)). The non-covalently adsorbed mAb (Peptide Mab: FNWYVDGVEVHNAK) as control peptide for the mAb adsorption, digestion and LC-MS experiment (right and left).

FIG. 6 left shows non-functionalized beads using the same sample processing steps as for the functionalized beads shown in FIG. 6 right. The non-functionalized beads are missing the bis-enol-ester moiety and therefore only having the residual alkyne moiety on its surface. The non-covalently adsorbed mAb peptide provides confidence in the whole workflow. The missing lipase peptides shows the ability of the washing protocol to remove non-covalently bound lipase.

FIG. 7: Schematic process for preparing a composition comprising a protein.

FIG. 7 shows an exemplary process or method for preparing a composition comprising a protein (protein of interest) according to the invention.

A typical process or method comprises at least 4 subsequent steps of (conventional) preparation and/or purification of a protein of interest, i.e. preparing a protein composition from the cell culture, followed by typically (at least) 3 ((conventional) chromatographic steps, optionally 4 (conventional) chromatographic steps.

Thus: after a protein composition has been prepared from the cell culture or culture supernatant (step 1), a process typically involves 3 subsequent (conventional) chromatographic steps, e.g. normally using 3 columns (optionally 4 columns) each employing a different purification principle (steps 2-4, or if an optional step 5 (column 4) is included, steps 2-5).

First, a composition from the cell culture (cells producing the protein/protein of interest) is prepared, e.g. by homogenizing the cells. The composition may also be a cell culture supernatant. Preferably, the above composition is subsequently purified, e.g. the composition then is a composition (as) separated (e.g. by filtration) from cells, cell debris and/or aggregates. Accordingly, the (subsequently purified) composition is a composition (as) separated (by filtration) from cells, cell debris and/or aggregates, e.g. is Harvest Cell Culture Fluid (HCCF).

Second, the above composition is further purified, e.g. by a further step of protein preparation and/or purification (step 2). The further step of protein preparation and/or purification preferably is affinity chromatography (preferably Protein A affinity chromatography). Hence, affinity chromatography (preferably Protein A affinity chromatography) is preferably the first chromatographic step. For example, the above composition (e.g. the Harvest Cell Culture Fluid (HCCF)) is subjected to Protein A affinity chromatography. In other words, the process includes the use of an affinity chromatography column (Protein A affinity chromatography column).

Third, the eluate is then subjected to a second column (Column 2/step 3).

Fourth, the eluate of the second column is then subjected to a third column (Column 3/step 4).

Fifth, if the quality (for example purity of the protein of interest) is not achieved or is to be further improved after the fourth step (i.e. after three columns (preferably each employing a different purification principle), optionally a fifth step (e.g. a fourth column with a fourth purification principle), can be used (Column 4/step 5). The skilled person can decide on a case by case basis whether a fifth step is necessary and/or beneficial e.g. when balancing costs and/or increased complexity versus improved quality.

Thus, steps 3 to 5 can be ion exchange chromatography (e.g. anion exchange chromatography (AEX) or cation exchange chromatography (CEX)), mixed mode chromatography and hydrophobic interaction chromatography (HIC) in any order. In other words, columns 2 to 4 can be ion exchange chromatography column (e.g. anion exchange chromatography (AEX) column or cation exchange chromatography (CEX) column), mixed mode chromatography column and hydrophobic interaction chromatography (HIC) column in any order, i.e., the process includes the use of any of the above columns in any order.

During development of a purification process the skilled person can determine the order of the steps/of the columns to be used.

Sixth, after Column 3 or the optional Column 4 (step 4 and, optionally step 5) the eluate is subjected to virus filtration and then, seventh, subjected to ultrafiltration/diafiltration (UFDF).

The process includes a step “(i) contacting a starting composition comprising a protein with a hydrolase inhibitor (such as Orlistat), wherein the hydrolase inhibitor is immobilized on a solid carrier” (e.g. the step is hydrolase inhibitor chromatography (a hydrolase inhibitor chromatographic step). In other words, the process includes the use of a hydrolase inhibitor (e.g. Orlistat) column.

Preferably, the hydrolase inhibitor chromatography/chromatographic step follows the step of affinity chromatography (preferably protein A affinity chromatography)/step 2. In other words, the process includes the use of a hydrolase inhibitor (e.g. Orlistat) column (directly) following the protein A column.

Alternatively, the hydrolase inhibitor chromatography/chromatographic step follows any one of steps 3 to 5 (steps 3 to 5 can be ion exchange chromatography (e.g. anion exchange chromatography (AEX) or cation exchange chromatography (CEX)), mixed mode chromatography and hydrophobic interaction chromatography (HIC) in any order). In other words, the process includes the use of a hydrolase inhibitor (e.g. Orlistat) column (directly) following the use of an ion exchange chromatography column (e.g. anion exchange chromatography (AEX) column or cation exchange chromatography (CEX) column), mixed mode chromatography column and hydrophobic interaction chromatography (HIC) column in any order.

The hydrolase inhibitor chromatography/chromatographic step normally does not (directly) follow the initial step of preparing the protein composition from the cell culture (e.g. does not follow HCCF). In other words, the hydrolase inhibitor chromatography/chromatographic step normally is not performed prior to affinity chromatography (e.g. protein A affinity chromatography)/step 2. This is because such an initial protein composition (e.g. protein composition from the cell culture/supernatant of the cell culture, preferably HCCF) typically still comprises a high degree of impurities, e.g. cells, cell debris and/or aggregates. Thus, the process normally does not include the use of a hydrolase inhibitor (e.g. Orlistat) column (directly) following the initial step of preparing the protein composition from the cell culture and/or does normally not include the use of a hydrolase inhibitor (e.g. Orlistat) column prior to the use of an affinity chromatographic column (e.g. protein A affinity chromatography column).

The Examples illustrate the invention.

### EXAMPLE 1: REDUCTION OF HYDROLYTIC ACTIVITY AND REMOVAL OF LPLA2 FROM A REC. LPLA2-SPIKED TOCILIZUMAB SOLUTION (5 MG/ML) USING ORLISTAT B IMMOBILIZED ON STREPTAVIDIN MAG SEPHAROSE BEADS

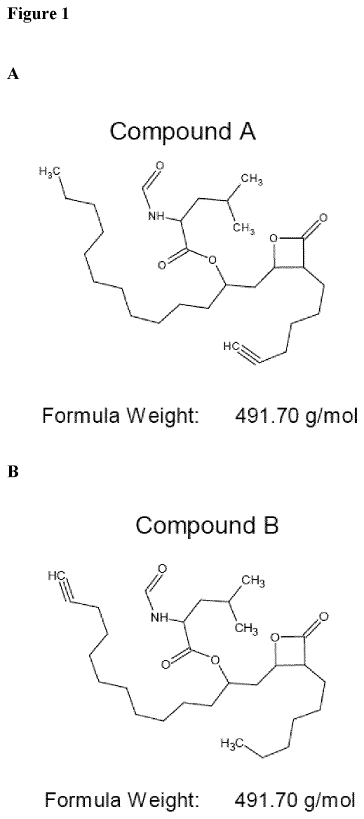

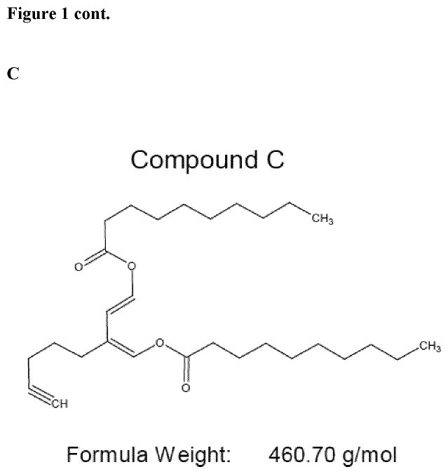

The following experiment demonstrates the removal of recombinant Lysosomal Phospholipase A2 (LPLA2) from a Tocilizumab solution using Orlistat B (formula (2) or compound (B)) (see FIG. 1B) immobilized on Streptavidin Magnetic Sepharose beads.

Functionalized beads were prepared by a two-step procedure comprising the conjugation of Orlistat B (ASM Research Chemicals GmbH) with azo biotin-azide (azo biotin-PEG3-azide) (Sigma-Aldrich; Order Number: 900891; CAS 1339202-33-3) per click-chemistry and a subsequent coupling step using Streptavidin Mag Sepharose beads (GE Healthcare) leveraging the high affinity of the attached biotin group to streptavidin.

The click reactions (sample/control) were performed by mixing reagents in reaction tubes according to the order indicated in Table 1

TABLE 1
Copper-catalyzed click-reaction composition for example 1.

| | Volume | |
|---|---|---|
| Reagent | [μl] | Composition |
| 1x PBS | 410 | 137 mM NaCl, 2.7 mM KCl, 10 mM |
| | | Na₂HPO₄, 1.8 mM KH₂PO₄, pH 7.4 |
| Orlistat B (sample) | 100 | 2 mM Orlistat B in DMSO (sample) |
| or DMSO (control) | | or DMSO (control) |
| azo biotin-azide | 30 | 5 mM Azo biotin-azide in DMSO (linker) |
| TCEP | 40 | 50 mM TCEP in PWA |
| TBTA | 120 | 1.7 mM TBTA in 4:1 t-butanol/DMSO |
| CuSO₄ | 40 | 50 mM CuSO₄ in PWA |

Both reaction mixtures, comprising either Orlistat or only DMSO for control beads, were incubated at room temperature for 2 h on a roller mixer and the solvent evaporated using a vacuum centrifuge (45° C., 2 h). The pellets were dissolved in 200 μL DMSO and stored at 4° C. until further use.

For the conjugation on Streptavidin Mag Sepharose Beads, 1 mL of 10% medium slurry was washed three times with 1 mL 1×PBS (137 mM NaCl, 2.7 mM KCl, 10 mM Na2HPO₄, 1.8 mM KH₂PO₄, pH 7.4). After washing, 1 mL of 10% medium slurry was mixed with 100 μL of the click reaction products (sample/control, each in duplicates) as described above and incubated for 1 h on a roller mixer. Conjugated beads were washed five times with 1 mL 1×PBS and stored at 4° C. until further use.

Tocilizumab was spiked with rec. LPLA2 to obtain a final antibody concentration of 5 mg/mL and LPLA2 concentration of 50 ppm (250 ng/mL) (Table 2):

TABLE 2
50 ppm LPLA2 was spiked in a 5 mg/ml Tocilizumab solution.

| V (Tocilizumab, | V (rec. LPLA2, | V (1 x PBS) | Total |
|---|---|---|---|
| c = 180 mg/mL) [μl] | c = 10 μg/mL) [μl] | [μl] | volume [μl] |
| 138.9 | 125 | 4736.1 | 5000 |

Per fishing reaction, 500 μL of LPLA2-spiked Tocilizumab was incubated with 200 μL of conjugated beads (sample/control) at 37° C. for 4 h in an Eppendorf ThermoMixer® at 900 rpm. After incubation, the supernatant (sample/control) was assessed by lipase activity assay (see section “Lipase activity assay” below) and the conjugated beads (sample/control) processed for on-bead digestion and LC-MS/MS analysis (see section “On-bead digestion and LC-MS/MS” below).

Lipase Activity Assay

This paragraph describes the lipase activity assays performed in Example 1, 2, 3 and 4. The lipase activity assay measures the lipase activity by monitoring the conversion of a non-fluorescent substrate (4-MUCA, Chem Impex Int'l Inc) to a fluorescent product (4-MU, Sigma-Aldrich) through the cleavage of the substrate ester bond. The assay reaction mixture contained 80 μL of reaction buffer (150 mM Tris-HCl pH 8.0, 0.25% (w/v) Triton X-100 and 0.125% (w/v) Gum Arabic), 10 μL 4-MUCA substrate (1 mM in DMSO), and 10 μL protein sample. Sample concentrations and corresponding buffers used in the corresponding examples are listed in Table 3.

TABLE 3
Summary of sample concentrations and corresponding buffers of
the antibody solution as used in the lipase activity assay.

| | | | | | | ¹Activities | |
|---|---|---|---|---|---|---|---|
| | | Assesed | | Assessed | | reported for/ | |
| | Protein to be | hydrolytic | | sample | | ²normalized | |
| Example | assessed | activity | Buffer | concentration | | to | |
| 1 | LPLA2 | Spiked | 1xPBS, pH 7.4 | 50 | ng/ml | 5 | ng/ml¹ |
| | | lipase | | | | | |
| 2 | LPLA2 | Spiked | 1xPBS, pH 7.4 | 50 | ng/ml | 5 | ng/ml¹ |
| | | lipase | | | | | |
| 3 | Glofitamab | endogenous | 50 mM Tris | 6.2-7.4 | mg/ml** | 100 | μg² |
| | | | Acetate, pH 7.0 | | | | |
| 4 | Trastuzumab | endogenous | 150 mM Tris, | 20 | g/l | 100 | μg² |
| | | | pH 8.0* | | | | |

*Trastuzumab conditioned protein A elution pool was rebuffered to 150 mM Tris-HCl pH 8.0 by using Amicon Ultra-0.5 ml centrifugal filter units (10,000 Da cut-off, Merck Millipore).
**Sample concentration of each sample was measured again after incubating the protein solution with the beads. This was performed in duplicate using a Thermo Scientific ™ NanoDrop ™ One Microvolume UV-Vis Spectrophotometer.

Each reaction was set up in technical triplicates in 96-well half-area polystyrene plates (black with lid and clear flat bottom, Corning Incorporated) and the increase of fluorescent signal (excitation at 355 nm, emission at 460 nm) was monitored every 10 min by incubating the reaction plate for two hours at 37° C. in an Infinite 200Pro plate reader (Tecan Life Sciences). MU production rate was derived from the slope of the fluorescent time course (0.5 hour-2 hour), and represents the raw rate of a reaction (k raw [RFU/h]).

An enzyme blank reaction was additionally set up to measure any non-enzymatic cleavage of the substrate caused by the buffer matrix. 10 μL protein sample were replaced by 10 μL of sample buffer (Table 3) in the reaction mixture. The self-cleavage rate (k self-cleavage [RFU/h]) was derived from the slope of the fluorescent time course (0.5 hour-2 hour). To convert the fluorescent signal (RFU) to M of MU, a standard MU triplicate was added per plate. 10 μL MU (100 μM in DMSO) were supplemented with 10 μL of sample buffer (Table 3) and 80 μL of reaction buffer. The conversion factor a [RFU/μM] was calculated by averaging the fluorescent signal (0.5 hour-2 hour) and dividing it by the final concentration of MU present in the well.

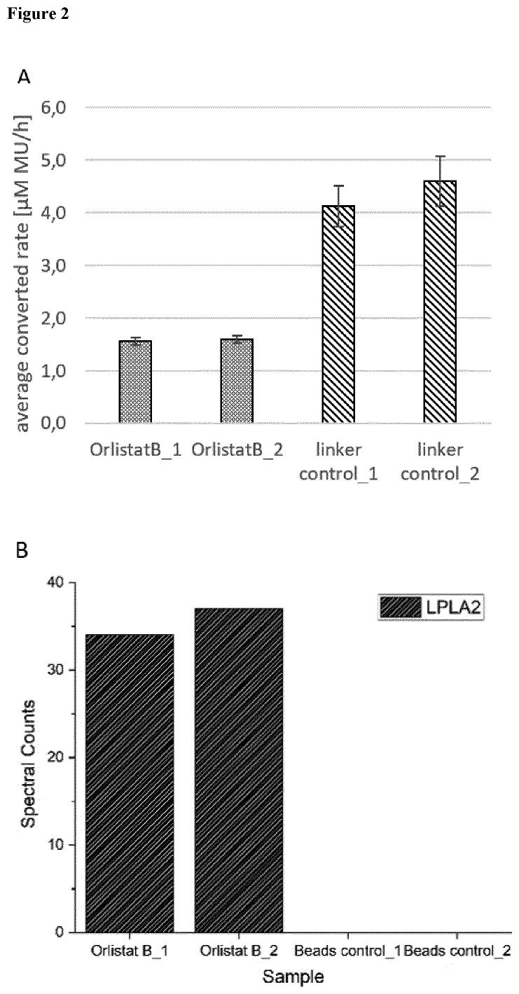

The lipase activity for a sample given in [μM MU/h] was determined by subtracting the reaction rate of the enzyme blank (k self-cleavage [RFU/h]) from the reaction rate of the sample (k raw [RFU/h]), and converting the fluorescent signal to μM MU/h by dividing the term by the conversion factor a [RFU/μM]. Activities were either reported for the lipase concentration applied per well (row 1 and 2 of Table 3) or normalized to 100 μg of antibody (row 3 and 4 of Table 3) (see also description of FIG. 2A).

On-Bead Digestion and LC-MS/MS

For on-bead digestion, the conjugated beads (sample, control) were consecutively washed three times with 1 mL of 1×PBS (137 mM NaCl, 2.7 mM KCl, 10 mM Na₂HPO₄, 1.8 mM KH₂PO₄), three times with 1 mL of 1% (w/v) SDS, three times with 1 mL of denaturation buffer (400 mM Tris/HCl, 8 M GdmCl, pH 8.5) and five times with 1 mL of 1×PBS (137 mM NaCl, 2.7 mM KCl, 10 mM Na₂HPO₄, 1.8 mM KH₂PO₄). The supernatant was discarded and the beads incubated in 300 μL of denaturation buffer (400 mM Tris/HCl, 8 M GdmCl, pH 8.5) and 10 μL of a DTT solution (0.1 g DTT/mL in PWA) for 60 min at 50° C. in an Eppendorf ThermoMixer® at 900 rpm. After incubation, 10 μL of an iodoacetic acid solution (0.33 g/mL in PWA) were added und incubated at room temperature for 30 min in the dark. Beads were washed with 1 mL of digestion buffer (0.1 M Tris/HCl, pH 7.0) and the supernatant was removed. After adding 250 μL of digestion buffer (0.1 M Tris/HCl, pH 7.0) and 5 μL of a 0.25 mg/mL trypsin solution (25 μg of lyophilized trypsin in 100 μl 10 mM HCl), an on-bead digestion was performed for 18 h at 50° C. in an Eppendorf ThermoMixer® at 900 rpm. The supernatant was transferred into a new reaction tube and the trypsin inactivated by adding 25 μL of a formic acid solution (10% in PWA) and the samples stored at 4° C. until analysis.

Peptides were separated at 60° C. on a CSH130 C18 (1.7 μm, 2.1 mm×150 mm) column (Waters) using a Thermo Fisher Scientific Vanquish-H UHPLC System. 100 μL of samples were injected at 300 μL/min flow rate. Mobile phase A consisted of 0.1% formic acid in water and mobile phase B consisted of 0.1% formic acid in acetonitrile. The 65 min gradient started with a linear increase of 0% to 40% B in 45 min, followed by a 5 min column wash at 95% B and column re-equilibration for 10 min at 1% B. Additionally, an eluent A run (65 min gradient) before and a 30 min wash gradient with 10% methanol after each sample was implemented to prevent carry over and distortion of results. The UHPLC system was coupled with a TripleTOF 6600 mass spectrometer (Sciex) with DuoSpray Ion Source. Tandem mass spectrometric analysis was performed using data dependent acquisition (DDA) with following settings: the top 20 most abundant ions were selected from every MS survey scan (250 ms) over a mass range of 200-2000 m/z. The MS/MS scans (69 ms) were acquired within a mass range of 100-1600 m/z (high sensitivity mode) including charge states of +2 to +5. For MS/MS selection, the dynamic exclusion was set for a period of 6 s and the precursor ion threshold to 150 counts per second.

The MS/MS data were searched against a customized CHO database with 35,862 protein sequences containing all CHO proteins from Uniprot.org (Swiss-Prot and TrEMBL annotations included), drug product sequences and human keratin impurities using the ProteinPilot software (Sciex, Version 5.0). Acceptance criteria for positive protein identification was set at a false discovery rate of <1% with at least two unique peptides at a confidence of 95%.

### Conclusion

Incubating a 5 g/l Tocilizumab solution spiked with 50 ppm rec. LPLA2 with immobilized Orlistat B significantly reduced the hydrolytic activity in the supernatant compared to the respective control without Orlistat B (Azo-Biotin-Azide coupled to Streptavidin Mag Sepharose beads) as shown by the Lipase activity assay (FIG. 2A). Consistent with these results, rec. LPLA2 could be identified by on-bead digestion and LC-MS/MS analysis solely on Orlistat B functionalized beads but not for the control beads, indicating covalent binding of Orlistat B on LPLA2 and consequently its removal from the antibody solution (FIG. 2B).

### EXAMPLE 2: REDUCTION OF HYDROLYTIC ACTIVITY FROM A REC. LPLA2-SPIKED TOCILIZUMAB SOLUTION (100 MG/ML) USING ORLISTAT B (FORMULA (2) OR COMPOUND B)) IMMOBILIZED ON STREPTAVIDIN MAG SEPHAROSE BEADS

This experiment demonstrates that the approach described in Example 1 is also applicable for antibody solutions containing a high protein concentration (i.e., 100 mg/mL Tocilizumab) at the same ratio of spiked rec. LPLA2 (i.e., 50 ppm, 5 μg/mL).

For this purpose, functionalized beads were prepared as described in Example 1.

Tocilizumab was spiked with rec. LPLA2 to obtain a final antibody concentration of 100 mg/mL and LPLA2 concentration of 50 ppm (5 μg/mL) (Table 4).

TABLE 4
50 ppm LPLA2 spike in 100 mg/ml Tocilizumab solution.

| V (Tocilizumab, | V (rec. LPLA2, | V (1 x PBS) | Total |
|---|---|---|---|
| c = 180 mg/mL) [μl] | c = 100 μg/mL) [μl] | [μl] | volume [μl] |
| 833 | 75 | 592 | 1500 |

250 μL of LPLA2-spiked Tocilizumab was incubated with 100 μL of conjugated beads (sample/control) at 37° C. for 4 h in an Eppendorf ThermoMixer® at 900 rpm. After incubation, the supernatant (sample/control) was assessed by lipase activity assay (see Example 1).

### Conclusions

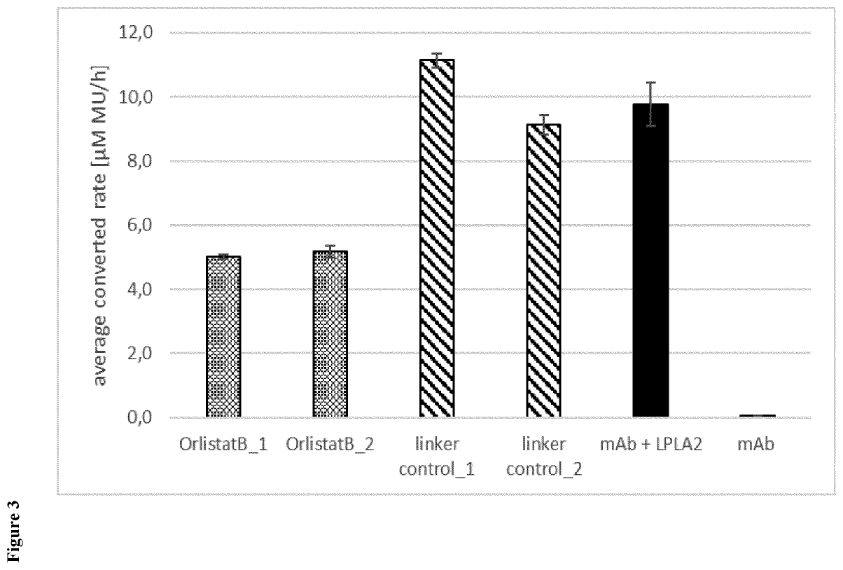

Consistent with the results from Experiment 1, incubating a 100 g/l Tocilizumab solution spiked with 50 ppm rec. LPLA2 with immobilized Orlistat B significantly reduced the hydrolytic activity in the supernatant compared to the respective control without Orlistat B (Azo-Biotin-Azide coupled to Streptavidin Mag Sepharose beads) as shown by the Lipase activity assay (FIG. 3A). This effect can be attributed to the immobilization of Orlistat B, since the control sample (Azo-Biotin-Azide coupled to Streptavidin Mag Sepharose beads) shows comparable hydrolytic activity as the antibody solution spiked with rec. LPLA2 (not comprising an incubation step with Streptavidin Mag Sepharose beads).

### EXAMPLE 3: REDUCTION OF HYDROLYTIC ACTIVITY FROM AN IN-PROCESS POOL OF GLOFITAMAB USING ORLISTAT B IMMOBILIZED ON STREPTAVIDIN SEPHAROSE® HIGH PERFORMANCE BEADS OR NHS-ACTIVATED SEPHAROSE BEADS

This experiment shows that the hydrolytic activity in a glofitamab in-process pool (i.e., MMAEX load solution) was reduced when incubated with Orlistat B (formula (2) or compound B)) immobilized either on NHS-activated Sepharose® 4 Fast Flow beads (GE Healthcare) or Streptavidin Sepharose® High Performance beads (GE Healthcare).

Preparation of Orlistat B Immobilized on NHS-Activated Sepharose® 4 Fast Flow Beads)

Functionalized Sepharose® 4 Fast Flow beads were prepared by a two-step procedure comprising the coupling of the linker Azido-PEG4-Amine (BroadPharm, BP-21615; CAS 951671-92-4) on the amine-reactive NHS-activated beads and, subsequently, the immobilization of Orlistat B by conjugating its alkyne group with the azido group of the bead-coupled linker per click chemistry.

For coupling the linker Azido-PEG4-Amine on the NHS-activated beads, the following steps were performed: 100 μL of Azido-PEG4-Amine was diluted in 1900 μL coupling buffer (0.2 M NaHCO₃, 0.5 M NaCl, pH 8.3) to obtain a total volume of 2000 μL. 600 μL of NHS-activated Sepharose® 4 Fast Flow beads was filled into a Poly-Prep Chromatography Column (BioRad) and washed with 10 CV of ice-cold 1 mM HCl (in PWA) solution immediately before use. 300 μL of the prepared Azido-PEG4-Amine solution were added to the washed beads and incubated for 4 h at room temperature on a roller mixer. For the blocking of unreacted NHS esters and washing, coupling buffer (0.2 M NaHCO₃, 0.5 M NaCl, pH 8.3), washing buffer A (0.1 M Na-acetate, 0.5 M NaCl, pH 4.0), washing Buffer B (0.1 M Tris-Cl, pH 8.0) and 1×PBS (137 mM NaCl, 2.7 mM KCl, 10 mM Na₂HPO₄, 1.8 mM KH₂PO₄, pH 7.4) were added in the indicated order and volumes:
a) 3×2CV blocking buffer
b) 3×2CV washing buffer A
c) 3×2CV blocking buffer
d) Leave for 30 min in blocking buffer
e) 3×2CV washing buffer A
f) 3×2CV washing buffer B
g) 3×2CV washing buffer A
h) 3×2CV washing buffer B
i) 3×2CV washing buffer A
j) 3×2CV washing buffer B
k) 5×2CV 1×PBS

For conjugating Orlistat B on the bead-coupled azido group of the linker per click chemistry the following steps were performed:

500 μL of the drained Azido-functionalized beads obtained as described above was mixed with 500 μL 1×PBS ((137 mM NaCl, 2.7 mM KCl, 10 mM Na₂HPO₄, 1.8 mM KH₂PO₄, pH 7.4) and homogenized. 400 μL of the resulting bead-slurry was filled into a new Poly-Prep Chromatography Column (BioRad). After settling down of the bead-bed, the supernatant was discarded. For conjugating Orlistat B (referred to as NHS Sepharose w/ Orlistat B) or Biotin-PEG4-Alkyne (referred to as NHS Sepharose w/o Orlistat B) (Sigma-Aldrich), the following reagents were added to the filled Poly-Prep Chromatography Column in the indicated order and volumes (Table 5):

TABLE 5
Copper-catalyzed click-reaction composition for functionalization
of Azido-functionalized beads in example 3

| | Volume | |
|---|---|---|
| Reagent | [μl] | Composition |
| 1x PBS | 150 | 137 mM NaCl, 2.7 mM KCl, 10 mM |
| | | Na₂HPO₄, 1.8 mM KH₂PO₄, pH 7.4 |
| Orlistat B | 50 | 2 mM Orlistat B in DMSO (sample) |
| (sample) or | | or 2 mM Biotin-PEG4-Alkyne in DMSO |
| Biotin-PEG4- | | (control) |
| Alkyne | | |
| (control) | | |
| TCEP | 20 | 50 mM TCEP in PWA |
| TBTA | 60 | 1.7 mM TBTA in 4:1 t-butanol/DMSO |
| CuSO₄ | 20 | 50 mM CuSO₄ in PWA |

Both reaction mixtures (sample/control) were incubated at room temperature for 2 h on a roller mixer and washed with 10 CV of 1×PBS (137 mM NaCl, 2.7 mM KCl, 10 mM Na₂HPO₄, 1.8 mM KH₂PO₄, pH 7.4). The columns containing the prepared resins were stored at 4° C. until further use.

Orlistat B Immobilized on Streptavidin Sepharose® High Performance Beads

For the preparation of Streptavidin Sepharose® High Performance beads functionalized with Orlistat B, the click reaction between Orlistat B and Biotin-PEG4-azide (BroadPharm; CAS 1309649-57-7), was performed by mixing the following reagents in reaction tubes according the indicated order and volumes (Table 6):

TABLE 6
Copper-catalyzed click-reaction composition for
functionalization of Streptavidin Sepharose ® High
Performance beads in Example 3.

| | Volume | |
|---|---|---|
| Reagent | [μl] | Composition |
| 1x PBS | 410 | 137 mM NaCl, 2.7 mM KCl, 10 mM |
| | | Na₂HPO₄, 1.8 mM KH₂PO₄, pH 7.4 |
| Orlistat B | 100 | 2 mM Orlistat B in DMSO (sample) |
| (sample) or | | or 2 mM Biotin-PEG4-Alkyne in DMSO |
| Biotin-PEG4- | | (control) |
| Alkyne | | |
| (control) | | |
| Biotin-PEG4- | 30 | 5 mM Biotin-PEG4-Azide (linker) |
| azide | | |
| TCEP | 40 | 50 mM TCEP in PWA |
| TBTA | 120 | 1.7 mM TBTA in 4:1 t-butanol/DMSO |
| CuSO₄ | 40 | 50 mM CuSO₄ in PWA |

Both reaction mixtures were incubated at room temperature for 2 h on a roller mixer and the volatile solvent evaporated using a vacuum centrifuge (45° C., 2 h) and stored at 4° C. until further use.

For the conjugation on Streptavidin Sepharose® High Performance beads, 1 mL of medium slurry was washed ten times with binding buffer (20 mM NaPO₄, 0.15 M NaCl, pH 7.5), filled into a Poly-Prep Chromatography Column (BioRad) and was equilibrated with 5 CV of binding buffer (20 mM NaPO₄, 0.15 M NaCl, pH 7.5). 500 μL of binding buffer (20 mM NaPO₄, 0.15 M NaCl, pH 7.5) was mixed with 200 μL of conjugated Biotin-Orlistat (referred to as Seph-SA w/ Orlistat B)/Biotin-Biotin (referred to as Seph-SA w/o Orlistat B) and the total mixture was added to the equilibrated beads. After incubation for 30 min at RT on a rolling mixer, the beads were successively washed with 10 CV of binding buffer (20 mM NaPO₄, 0.15 M NaCl, pH 7.5) and 10 CV of 1×PBS (137 mM NaCl, 2.7 mM KCl, 10 mM Na₂HPO₄, 1.8 mM KH₂PO₄, pH 7.4). Conjugated beads (Seph-SA w/ Orlistat B and Seph-SA w/o Orlistat B) were stored at 4° C. until further use.

Incubation of Conjugated Beads with a MMAEX Load Solution of the Antibody Glofitamab

100 μL of the functionalized bead slurries (NHS Sepharose w/ Orlistat B, NHS Sepharose w/o Orlistat B, Seph-SA w/ Orlistat B and Seph-SA w/o Orlistat B) as well as the plain bead slurries (NHS-activated Sepharose beads and Streptavidin Sepharose® High Performance) were transferred into a reaction tube and 50 μL of the supernatant discarded. 450 μL of MMAEX load solution of Glofitamab (c=7.3 mg/ml) was added and the mixture incubated at 25° C. in an Eppendorf Thermomixer at 850 rpm. The samples were centrifuged for 30 s at 1000 rpm and the supernatants were assessed for lipase activity as described in example 1.

### Conclusions

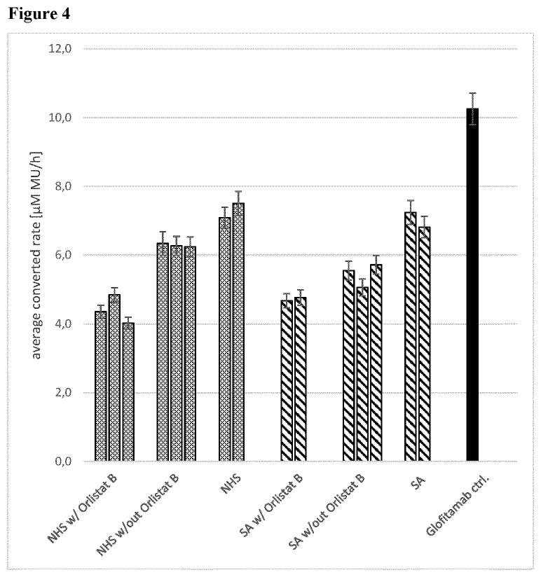

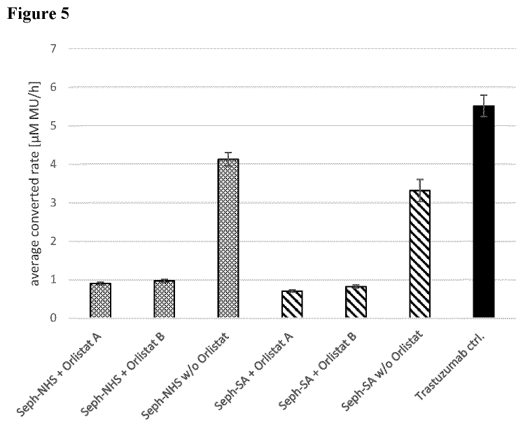

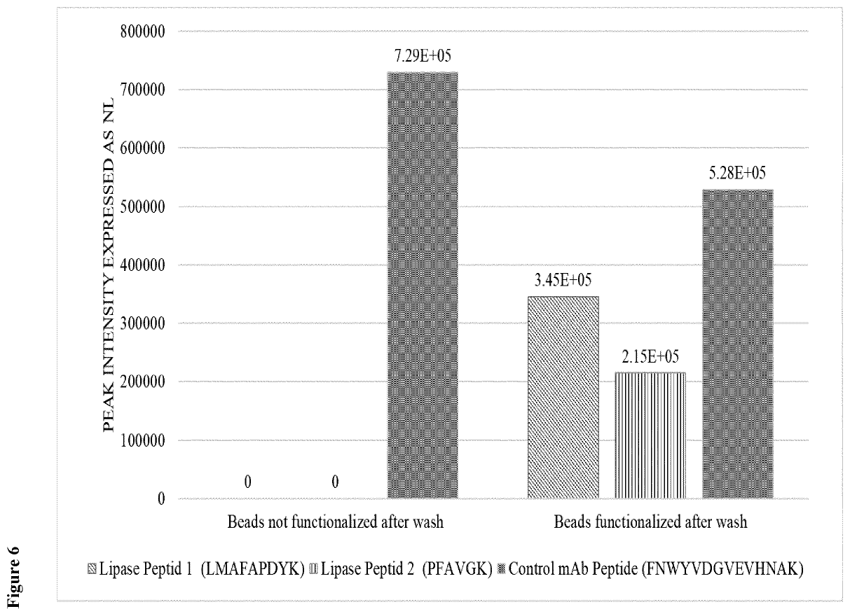

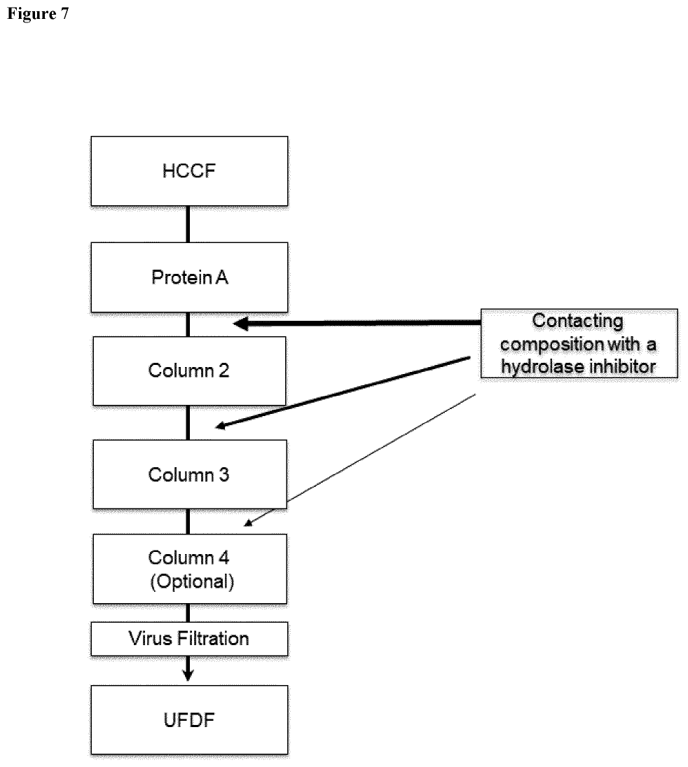

Incubation of Glofitamab (MMAEX load solution) with Orlistat B functionalized beads resulted in a significant reduction of the hydrolytic activity, as can been seen by the reduced average converted rates of these beads in comparison to the respective control beads (i.e., conjugated beads without Orlistat B and unconjugated beads) and the starting protein composition in FIG. 4. These data indicate the capability of immobilized Orlistat B to remove hydrolytically active enzymes from an in-process pool of an antibody solution featuring a considerable level of hydrolytic activity. Furthermore, the beneficial effect of immobilized Orlistat B could be demonstrated for both NHS-activated Sepharose® 4 Fast Flow beads (coupling via NHS and click chemistry as described above) and Streptavidin Sepharose® High Performance beads (GE Healthcare) (coupling via click chemistry and leveraging the high affinity between biotin and streptavidin as described above), since both approaches resulted in a meaningful reduction of the hydrolytic activity compared to the respective controls (i.e., conjugated beads without Orlistat B, unconjugated beads and the starting protein composition) as shown by the average converted rates in FIG. 4.

### EXAMPLE 4: REDUCTION OF HYDROLYTIC ACTIVITY FROM AN IN-PROCESS POOL OF TRASTUZUMAB USING ORLISTAT A OR ORLISTAT B IMMOBILIZED ON STREPTAVIDIN SEPHAROSE® HIGH PERFORMANCE BEADS OR NHS-ACTIVATED SEPHAROSE BEADS

This experiment demonstrates that the hydrolytic activity in a Herceptin® (Trastuzumab) in-process pool (i.e., protein A elution pool) was reduced by using either Orlistat A (formula (1) or compound A)) or Orlistat B (formula (2) or compound B) immobilized either on NHS-activated Sepharose® 4 Fast Flow beads or Streptavidin Sepharose® High Performance beads (GE Healthcare) beads.

Functionalized beads (NHS-activated Sepharose® 4 Fast Flow and Streptavidin Sepharose® High Performance beads (GE Healthcare)) were prepared for both Orlistat B and Orlistat A (by substituting Orlistat B with Orlistat A) as described in Example 3.

100 μL of the Orlistat A/B functionalized beads/corresponding control beads (for both NHS-activated Sepharose® 4 Fast Flow beads as well as Streptavidin Sepharose® High Performance beads, which were processed as described above) were transferred into a reaction tube and 50 μL of the supernatant discarded. 450 μL of protein A elution pool of Herceptin (7 g/l) was added and the mixture incubated 25° C. in a Thermomixer at 850 rpm. The samples were centrifuged for 30 s at 1000 rpm and the supernatants as well as the starting protein composition were assessed by lipase activity assay as described in Example 1.

### Conclusions

A significant reduction of hydrolytic activity could be achieved for protein A elution pool of Herceptin after incubation with both Orlistat A and Orlistat B immobilized on either NHS-activated Sepharose 4 Fast Flow beads or Streptavidin Sepharose® High Performance beads compared to the respective controls and the starting protein composition (not comprising an incubation step with functionalized beads) as shown by the average converted rates in FIG. 5, indicating the capability of immobilized hydrolase inhibitors (i.e. Orlistat A and B) to remove hydrolytically active enzymes from an already purified antibody solution (i.e. In-process pool of trastuzumab).

### EXAMPLE 5: PROTEOMICS ASSESSMENT OF THE LIPASE BIS-ENOL-ESTER CONSTRUCT BOUND TO A SOLID SUPPORT

This experiment demonstrates the ability of a bis-enol-ester (formula (3) or compound C), covalently coupled on azide modified magnetic particles (via Click reaction chemistry), to covalently and selectively bind a lipase (model lipase: CalB2) and remove the respective lipase from an antibody solution. The ability of the functionalized beads to bind the lipase was compared to non-functionalized beads as control.

Test sample: Herceptin (mAb; 0.2 μg/μl in Na-Phosphate-Buffer) as control protein for the LC-MS based proteomics workflow was mixed with the lipase CalB2 (CalB2; 0.2 μg/l in Na-Phosphate-Buffer) in a ca. 1:1 (mol/mol) ratio) as target. The above-mentioned samples were addressed with compound C (bis-enol-ester) covalently coupled to 60 μg azide modified magnetic particles (60 μl of a 1 μg/l aqueous solution were prepared) (Polystyrene based azide beads; CLK-1036-1, Jena-Bioscience) which were made by click reaction and purification (see protocol experiment Example 1 and general procedure described for the Jena-Bioscience click protocol). As control non-functionalized azide modified beads (Polystyrene based azide beads; CLK-1036-1, Jena-Bioscience) have been treated in the same way as the compound C coupled beads except that the coupling step was performed solely with the solvent of compound C.

Incubation of the CalB2 and mAb mixture was done by mixing 60 μg (60 μl of a 1 μg/μl solution) of the respective functionalized or non-functionalized beads with 50 μl CalB2 (0.2 μg/μl) and 50 μl mAb (0.2 μg/μl). The samples were vortexed with 1000 rpm over night at 40° C. After incubation, the functionalized beads as well as the non-functionalized beads have been washed according the following protocol using magnetic separation and low-bind disposables:
1^{st} wash: The 60 μg beads were washed with 200 μl PBS buffer two times followed by drying step
2^{nd} wash: The dried beads from the 1^{st} step were washed three times with PBS-buffer containing Triton-X100 (10 μl Triton-X100 with 9990 μl PBS diluted) followed by air drying at room temperature
3^{rd} wash: The beads from step two are washed two times with 200 μl water followed by air drying at room temperature step
4^{th} wash: The beads from step 3 are washed with 200 μl (70% Acetonitrile/30% water and 0.1% formic acid (v/v/v)
5^{th} wash: Beads from step 4 have been finally washed with 200 μl water For the targeted LC-MS analyses the washed beads were Carbamidomethylated with 5 μl IAM-Solution (55 mM: 1 mg/ml in 98.3 μl MilliQ-Water) and reduced with 5 μl DTT (10 mM in Water) solution for 30 min.

After the above mentioned steps, denaturing of the proteins on the respective beads was done by addition of 50 μl denaturation buffer/digest buffer (Trypsin dilution (50 mM AcOH), 10 μl AcOH, glacial with 3470 μL MilliQ-Water diluted, 4° C. followed by 20 μg trypsin are dissolved in the original tube in 40 μl trypsin dilution solution (50 mM AcOH)). 5 μl of the trypsin solution, 1 μl Rapid PNGase F and 1 μl (N/O—) deglycosylation MIX II were added to the sample mix followed by mixing and incubation: (16-20 hours at 37° C. in a thermoshaker at 1000 rpm, 37° C.). Bead supernatant separation was done by brief centrifugation followed by magnetic separation.

The digest was stopped by addition of 2 μl FA-Solution to the sample digest followed by incubation step (1.5-2 h at 37° C. in a thermal shaker at 800 rpm (for hydrolysis of PPS)). Storage of the digest samples at −80° C. (at the original samples) until analysis

The targeted LC-MS analysis was performed using Water/Acetonitrile gradients with TFA (0.05% v(v) as buffer. The separation column was a PepSwift Monolithic Capillary Column, 200 μm×5 cm, P/N: 161409. As mass spectrometer, a Thermo LTQ FT system was used. Further experimental design for quantitative analyses followed general experimental targeted LC-MS procedures. The proteins bound to the beads were applied to the LC-MS system by injection of 1 μl of the digested sample on the analytical column.

The following peptides and respective masses have been used for evaluation of the absolute peak intensity resulting from targeted mass spectrometry readout (Peak Intensity's as normalized level ((NL)):
Peptide CalB2 Lipase 1: LMAFAPDYK, charge z=2; mass/charge: 528.27 Da
Peptide CalB2 Lipase 2: PFAVGK, charge z=2; mass/charge: 309.68 Da
Peptide mAb: FNWYVDGVEVHNAK, charge z=3; mass/charge: 559.94 Da

Respective targeted LC-MS signals for the mAb peptide and Lipase peptides have been used with their respective signal intensity to generate FIG. 6.

The analysis of the mass spectrometry results showed that both selective peptides for the lipase CalB2 can be found after digestion with intense signal counts.

Thus, the experiment clearly demonstrates that the model lipase CalB2 binds to beads functionalized with bis-enol ester even when subjected to extensive washing steps (FIG. 6). This interaction was clearly specific since no lipase was detected on the non-functionalized control beads.

The mAb is highly prone to precipitation and is adsorbed to the beads in a non-covalent manner. The mAb peptide was detected on functionalized and on control beads verifying a functional LC-MS based proteomics workflow for both the functionalized beads and the control beads.

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

1. A method comprising the steps of
(i) contacting a starting composition comprising a protein with a hydrolase inhibitor, wherein the hydrolase inhibitor is immobilized on a solid carrier;
(ii) recovering a composition comprising the protein.

2. The method of claim 1, wherein the method is for preparing a composition comprising a protein.

3. The method of claim 1 or 2, wherein the composition comprising the protein has reduced hydrolytic activity compared to the starting composition and/or has a reduced content of a hydrolase compared to the starting composition, for example, as determined by a lipase activity assay (lipase enzymatic assay for polysorbates (LEAP) assay) and/or a fatty acid by mass-spectrometry (FAMS) assay.

4. The method of any one of claims 1 to 3, wherein the method is for reducing the hydrolytic activity in the starting composition and/or for reducing the content of a hydrolase in the starting composition.

5. The method of any one of claims 1 to 4, wherein the starting composition further comprises (a) hydrolase(s) and/or has hydrolytic activity.

6. The method of any one of claims 1 to 5, wherein the protein composition is prepared by removing a hydrolase from the starting composition and/or by reducing the content of a hydrolase in the starting composition.

7. The method of any one of claims 1 to 6, wherein said step (i) further comprises a step (a) adsorbing the hydrolase to the hydrolase inhibitor.

8. The method of any one of claims 1 to 7, wherein the composition comprising a protein is a solution comprising a protein.

9. The method of claim 8, wherein the solution is an aqueous solution.

10. The method of claim 8 or 9, wherein the solution is a buffered solution.

11. The method of any one of claims 1 to 10, wherein the hydrolase(s) is/are (an) esterase(s) and/or (a) amidase(s).

12. The method of claim 11, wherein the esterase(s) is/are (a) carboxylic ester hydrolase(s) and/or (a) thioesterase(s).

13. The method of claim 12, wherein the carboxylic ester hydrolase(s) is/are (a) lipase(s).

14. The method of any one of claims 1 to 12, wherein the hydrolase(s) is/are selected from the group consisting of Lipoprotein Lipase, Palmitoyl Proteinthioesterase, Acid Ceramidase, the C-terminal domain of Fatty Acid synthase, Putative Phospholipase b-like 2, Lysosomal Acid Lipase and Lysosomal Phospholipase.

15. The method of any one of claims 1 to 14, wherein the immobilized inhibitor is selected from the group consisting of orlistat or bis-enol-ester.

16. The method of any one of claims 1 to 15, wherein the solid carrier is selected from the group consisting of sepharose, polystyrene and smart polymers.

17. The method of any one of claims 1 to 16, wherein the inhibitor is immobilized on a solid carrier via the reaction of an azido group and an alkyne group.

18. The method of any one of claims 1 to 16, wherein the inhibitor is immobilized on a solid carrier via the binding of a streptavidin group to a biotin group.

19. The method of any one of claims 1 to 16, wherein the inhibitor is immobilized on a solid carrier via the reaction of an amino group and an N-Hydroxysuccinimid group.

20. The method of any one of claims 1 to 19, wherein the inhibitor is a group obtainable by reacting a compound comprising or consisting of formula (1), (2), (3) or (4), preferably formula (1), (2) or (4) with an azide

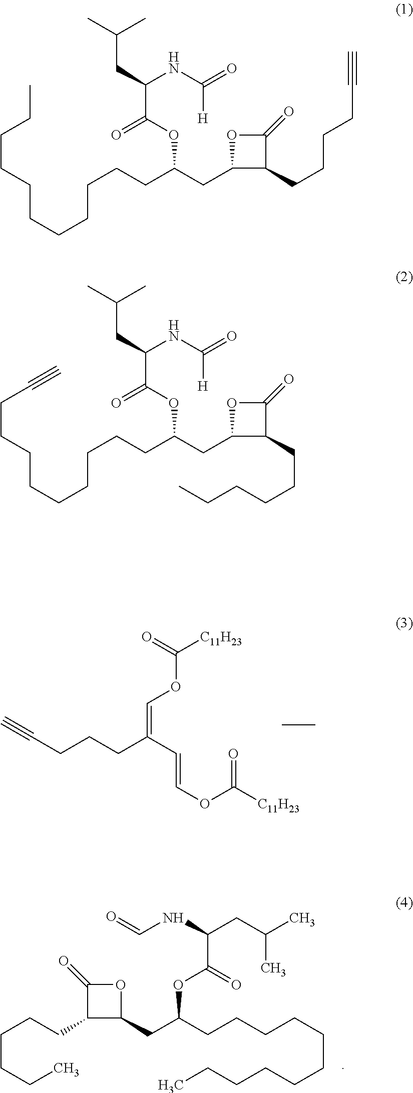

21. The method of any one of claims 1 to 20, wherein the steps are carried out in the following order: step (i) followed by step (ii).

22. The method of any one of claims 1 to 21, further comprising, prior to and/or after step (i) and/or prior to and/or after step (ii), the steps of protein preparation and/or purification.

23. The method of any one of claims 1 to 22, wherein step (i) is carried out after affinity chromatography, preferably after a Protein A affinity chromatography.

24. The method of any one of claims 1 to 23, wherein the protein is an antibody.

25. The method of claim 24, wherein the antibody is a monoclonal antibody.

26. The method of claim 24 or 25, wherein the antibody is a human or a humanized antibody.

27. The method of any one of claims 1 to 26, wherein the protein is an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A antibody, an antiVEGF-A/ANG2 antibody, an anti-CD79b antibody, an anti-ST2 antibody, an anti-factor D antibody, an anti-factor IX antibody, an anti-factor X antibody, an anti-abeta antibody, an antitau antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, a FAP-IL2v fusion protein, ocrelizumab, pertuzumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, lebrikizumab, omalizumab ranibizumab, emicizumab, selicrelumab, prasinezumab, glofitamab, simlukafusp alfa, and RG7827.

28. A method for preparing a protein formulation, the method comprising the steps of the method of any one of claims 1 to 27, wherein the method for preparing the protein formulation further comprises adding a surfactant, preferably a fatty acid ester, to the composition.

29. A method for preparing a protein formulation, wherein the method for preparing the protein formulation further comprises adding a surfactant, preferably a fatty acid ester, to the composition obtained by or obtainable by the method of any one of claims 1 to 27.

30. The method of claim 28 or 29, wherein the protein formulation is (essentially) free of visible and/or subvisible particles.

31. The method of any one of claims 28 to 30, wherein the protein formulation is stable for at least 6 months, at least 12 months, at least 18 months, at least 24 months, at least 36 months or at least 60 months under the recommended storage conditions.

32. The method of any one of claims 28 to 30, wherein the fatty acid ester is polyoxyethylene sorbitan or iso-sorbide fatty acid mono-, di- or tri-ester.

33. The method of any one of claims 28 to 32, wherein the fatty acid ester is Polyoxyethylene (20) sorbitan laurate.

34. The method of any one of claims 28 to 32, wherein the fatty acid ester is selected from the group group consisting of polysorbate 20, polysorbate 40, polysorbate 60, polysorbate 61, polysorbate 65, polysorbate 80, polysorbate 81, polysorbate 85 or polysorbate 120, or a combination thereof.

35. The method of any one of claims 28 to 32 wherein the fatty acid ester is polysorbate 20 or polysorbate 80.

36. The method of any one of claims 28 to 31 wherein the surfactant is Mono- or Di-acylglycerol, saccharide-fatty acid ester or α-tocopheryl Polyethylene glycol (PEG) succinate.

37. The method of any one of claims 28 to 36, wherein the degradation of the fatty acid ester is less than 20%, preferably less than 10% and more preferably less than 5% within 24 months.

38. The method of any one of claims 26 to 37, wherein the antibody concentration is at least 1 mg/ml and up to 250 mg/ml.

39. The method of any one of claims 28 to 38, further comprising adding a buffer, excipient, diluent, stabilizer and/or carrier to the composition.

40. The method of any one of claims 28 to 39, wherein the protein formulation is a pharmaceutical composition.

41. The method of any one of claims 1 to 40, wherein the composition comprising the protein and/or the protein formulation is essentially free of hydrolase inhibitors.

42. A protein composition obtained by or obtainable by the method of any one of claims 1 to 27.

43. A protein formulation obtained by or obtainable by the method of any one of claims 28 to 41.

44. The protein formulation of claim 43 for use as a medicament or for use in medicine.

45. Use of a hydrolase inhibitor immobilized on a solid carrier for preparing a protein composition and/or a protein formulation.

46. Use of a hydrolase inhibitor immobilized on a solid carrier for removing or reducing hydrolytic activity in a protein composition and/or in a protein formulation.

47. Use of a hydrolase inhibitor immobilized on a solid carrier for removing or reducing (content of) impurities, particularly of host cell proteins, and preferably of (a) hydrolase(s) in a protein composition and/or in a protein formulation.

48. Use of a hydrolase inhibitor immobilized on a solid carrier for inhibiting or reducing (the formation of) visible and/or sub-visible particles and/or the occurrence/presence of surfactant degradants in a protein composition and/or in a protein formulation.

49. A method for immobilizing a hydrolase inhibitor on a solid carrier, comprising the steps:
providing a solid carrier,
providing a hydrolase inhibitor,
contacting the solid carrier with a solution containing the hydrolase inhibitor, and
allowing the hydrolase inhibitor to be immobilized on the solid carrier.

50. The method according to claim 49, wherein the solid carrier is selected from poly(meth)acrylates like polymethylmethacrylate (PMMA), polystyrene, polyethylene oxide, cellulose and cellulose derivatives, e.g., cellulose acetate (CA) or regenerated cellulose, agarose, including crosslinked agarose, polysulfone (PSU), polyethersulfone (PES), polyethylene (PE), polypropylene (PP), polycarbonate (PC), polyacrylonitrile (PAN), polyamide (PA), polytetrafluoroethylene (PTFE), and blends or copolymers of the foregoing, or blends or copolymers with hydrophilizing polymers, preferably with polyvinylpyrrolidone (PVP) or polyethyleneoxide (PEO).

51. The method according to claim 49 or 50, wherein the step of providing the solid carrier includes a step of treating the solid carrier to introduce functional groups preferably selected from hydroxyl groups, carboxylate groups, ketones, aldehydes, isocyanates, epoxides, hydroxyl groups, carboxylate groups, amines, alkynes, azides, and biopolymers (e.g. streptavidin and/or biotin).

52. The method according to claim 51, wherein the step of providing the hydrolase inhibitor includes a step of treating the hydrolase inhibitor to introduce functional groups capable of reacting with the functional groups of the solid carrier to preferably form a streptavidin-biotin interaction or to form one or more groups selected from esters, amides, imines, urethanes, ureas, β-amino alcohols and 1,2,3-triazoles.

53. The method according to any one of claims 49 to 52, wherein the step of providing the hydrolase inhibitor includes a step of treating the hydrolase inhibitor to introduce functional groups preferably selected from hydroxyl groups, carboxylate groups, ketones, aldehydes, isocyanates, epoxides, hydroxyl groups, carboxylate groups, amines, alkynes, azides, and biopolymers (e.g. streptavidin and/or biotin).

54. The method according to claim 53, wherein the step of providing the solid carrier includes a step of treating the solid carrier to introduce functional groups capable of reacting with the functional groups of the hydrolase inhibitor to preferably form a streptavidin-biotin interaction or to form one or more groups selected from esters, amides, imines, urethanes, ureas, β-amino alcohols and 1,2,3-triazoles.

55. The method according to any one of claims 49 to 53, wherein the method comprises the steps:
providing a solid carrier,
contacting the solid carrier with a solution containing a linker to form a carrier-linker,
providing a hydrolase inhibitor,
contacting the carrier-linker with a solution containing the hydrolase inhibitor, and
allowing the hydrolase inhibitor to be immobilized on the carrier-linker.

56. The method according to claim 55, wherein the step of providing the solid carrier includes a step of treating the solid carrier to introduce functional groups preferably selected from hydroxyl groups, carboxylate groups, ketones, aldehydes, isocyanates, epoxides, hydroxyl groups, carboxylate groups, amines, alkynes, azides and biopolymers (e.g. streptavidin and/or biotin).

57. The method according to claim 55 or 56, wherein the linker comprises functional groups capable of reacting with the functional groups of the solid carrier to preferably form a streptavidin-biotin interaction or to form one or more groups selected from esters, amides, imines, urethanes, ureas, β-amino alcohols and 1,2,3-triazoles.

58. The method according to any one of claims 55 to 57, wherein the step of providing the hydrolase inhibitor includes a step of treating the hydrolase inhibitor to introduce functional groups preferably selected from hydroxyl groups, carboxylate groups, ketones, aldehydes, isocyanates, epoxides, hydroxyl groups, carboxylate groups, amines, alkynes, azides and biopolymer (e.g streptavidin and/or biotin).

59. The method according to any one of claims 55 to 58, wherein the linker further comprises functional groups capable of reacting with the functional groups of the hydrolase inhibitor to preferably form a streptavidin-biotin interaction or to form one or more groups selected from esters, amides, imines, urethanes, ureas, β-amino alcohols and 1,2,3-triazoles.

60. The method according to any one of claims 49 to 59, wherein the method comprises the steps:
providing a solid carrier,
providing a hydrolase inhibitor,
contacting the hydrolase inhibitor with a linker to form a hydrolase inhibitor-linker,
contacting the solid carrier with a solution containing the hydrolase inhibitor-linker, and
allowing the hydrolase inhibitor-linker to be immobilized on the solid carrier.

61. The method according to claim 60, wherein the step of providing the solid carrier includes a step of treating the solid carrier to introduce functional groups preferably selected from hydroxyl groups, carboxylate groups, ketones, aldehydes, isocyanates, epoxides, hydroxyl groups, carboxylate groups, amines, alkynes, azides and biopolymers (e.g streptavidin and/or biotin).

62. The method according to claim 60 or 61, wherein the linker comprises functional groups capable of reacting with the functional groups of the solid carrier to preferably form a streptavidin-biotin interaction or to form one or more groups selected from esters, amides, imines, urethanes, ureas, β-amino alcohols and 1,2,3-triazoles.

63. The method according to any one of claims 60 to 62, wherein the step of providing the hydrolase inhibitor includes a step of treating the hydrolase inhibitor to introduce functional groups preferably selected from hydroxyl groups, carboxylate groups, ketones, aldehydes, isocyanates, epoxides, hydroxyl groups, carboxylate groups, amines, alkynes, azides and biopolymers (e.g. streptavidin and/or biotin).

64. The method according to any one of claims 60 to 63, wherein the linker further comprises functional groups capable of reacting with the functional groups of the hydrolase inhibitor to preferably form a streptavidin-biotin interaction or to form one or more groups selected from esters, amides, imines, urethanes, ureas, β-amino alcohols and 1,2,3-triazoles.

65. The method according to any one of claims 49 to 64, wherein the functional groups on the solid carrier comprise amino groups, preferably primary amino groups.

66. The method according to claim 65, wherein the amino groups on the solid carrier are introduced by treatment with a reactive plasma, preferably a plasma generated from a gas mixture comprising ammonia.

67. The method according to any of claims 55 to 66, wherein the linker is a compound containing at least two functional groups selected from hydroxyl groups, carboxylate groups, ketones, aldehydes, isocyanates, epoxides, hydroxyl groups, carboxylate groups, amines, alkynes, azides and biopolymers (e.g. streptavidin and/or biotin).

68. The method according to claim 67, wherein the linker furthermore contains a polyoxyethylene or polyoxypropylene moiety, preferably containing 3 to 20 (more preferably 3 to 10, even more preferably 3 to 5) oxyethylene or oxypropylene units, to which the at least two functional groups are bound.

69. The method according to any one of claims 55 to 68, wherein the linker contains at least two different functional groups selected from hydroxyl groups, carboxylate groups, ketones, aldehydes, isocyanates, epoxides, hydroxyl groups, carboxylate groups, amines, alkynes, azides and biopolymers (e.g. streptavidin and/or biotin).

70. The method according to claim 69, wherein the linker contains per molecule a first functional group selected from hydroxyl groups, carboxylate groups, ketones, aldehydes, isocyanates, epoxides, hydroxyl groups, carboxylate groups, amines, alkynes, azides, streptavidin and biotin and furthermore at least two second functional groups, the at least two second functional groups preferably being of the same type, selected from hydroxyl groups, carboxylate groups, ketones, aldehydes, isocyanates, epoxides, hydroxyl groups, carboxylate groups, amines, alkynes, azides and biopolymers (e.g. streptavidin and/or biotin) which are different from the first functional group.

71. The method according to any one of claims 49 to 70, wherein the solid carrier is crosslinked agarose, preferably sepharose, more preferably Mag-Sepharose-Streptavidin (beads) or Streptavidin Sepharose® High Performance (beads).

72. The method according to any one of claims 49 to 71, wherein the solid carrier contains a functional group including streptavidin and the linker contains a functional group including biotin.

73. The method according to claim 72, wherein the linker furthermore contains an azide group and the hydrolase inhibitor contains an alkyne group.

74. The method according to any of claims 49 to 73, wherein the solid carrier contains a functional group including a carboxylate, which is optionally NHS-activated, and the linker contains a functional group including an amine.

75. The method according to claim 74, wherein the linker furthermore contains an azide group and the hydrolase inhibitor contains an alkyne group.

76. The method according to any one of claims 49 to 75, wherein the linker is selected from Biotin-PEG3-Azide (CAS 875770-34-6), Biotin-PEG4-Azide (CAS 1309649-57-7), Azo-Biotin-Azide (CAS 1339202-33-3) and Azido-PEG4-Amine (CAS 951671-92-4).

77. The method according to any one of claims 49 to 76, wherein the hydrolase inhibitor contains an alkyne group and is selected from compounds having a structure selected from: 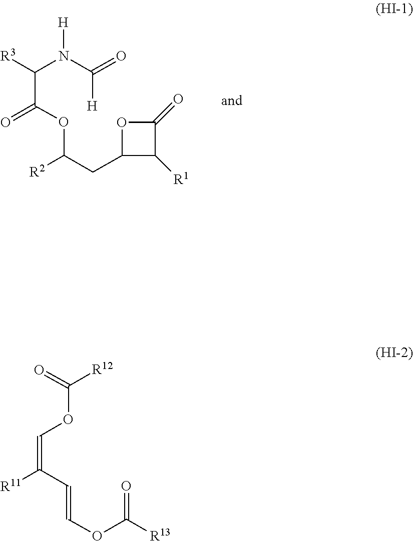
wherein in (HI-1)
R¹, R² and R³ are each independently selected from C₂₋₂₄-alkyl which may be linear or branched, wherein at least one terminal —CH₂—CH₃ group in R¹, R² or R³ is replaced by a —C≡CH group,
wherein in (HI-2)
R¹¹, R¹² and R¹³ are each independently selected from C₂₋₂₄-alkyl which may be linear or branched, and wherein at least one terminal —CH₂—CH₃ group in R¹¹, R¹² and R¹³ is replaced by a —C≡CH group.

78. The method according to claim 77, wherein the number of carbon atoms in R¹, R² and R¹¹ is independently selected from 2 to 15, preferably 3 to 14, more preferably 4 to 12, even more preferably from 5 to 11.

79. The method according to claim 77 or 78, wherein the number of carbon atoms in R¹² and R¹³ is independently selected from 5 to 24, preferably 8 to 18, more preferably 10 to 16, even more preferably from 10 to 12.

80. The method according to any one of claims 77 to 79, wherein the number of carbon atoms in R³ is selected from 2 to 10, preferably 2 to 8, more preferably 3 to 6, even more preferably from 3 to 5.

81. The method according to any one of claims 77 to 80, wherein the alkyl groups of R¹ and R² are linear.

82. The method according to any one of claims 77 to 81, wherein the alkyl groups of R¹¹ R¹² and R¹³ are linear.

83. The method according to any one of claims 77 to 82, wherein the alkyl group of R³ is branched.

84. The method according to any one of claims 77 to 83, wherein formula (HI-1) has the following structure (HI-1a): 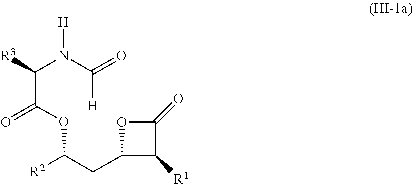
wherein R¹, R² and R³ are as defined in any one of claims 77, 78, 80, 81 and 83.

85. The method according to any one of claims 49 to 84, wherein the hydrolase inhibitor contains an alkyne group and is selected from compounds having a structure selected from:

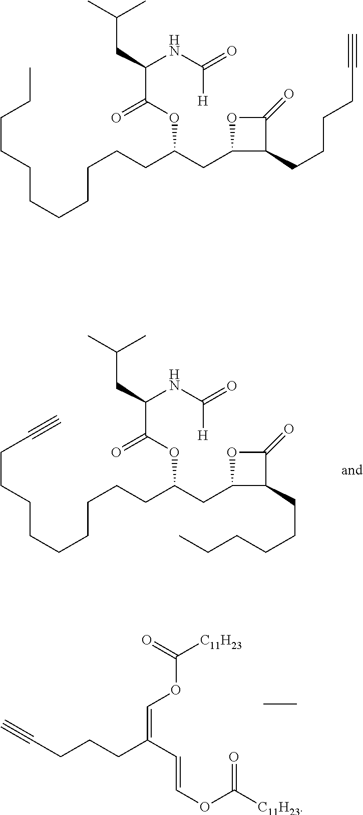

86. A hydrolase inhibitor immobilized on a solid carrier which is obtainable by the method according to any one of claims 49 to 85.

87. A device comprising the hydrolase inhibitor immobilized on a solid carrier according to claim 86.

88. The device according to claim 87, wherein the device is a tubular device having at least two openings.

89. The device according to claim 87 or 88, wherein the device is comprised of a container, preferably made of a metal, polymer or glass, the container forming a cavity in which the hydrolase inhibitor immobilized on a solid carrier is contained.

90. The device according any one of claims 87 to 89, wherein the device is a column, which is at least partially filled with the hydrolase inhibitor immobilized on a solid carrier.
